SEQUENCE LISTING (76 sequences)

SEQ ID NO: 1 - DNA, 20 bp, Artificial Sequence
  Synthetic oligonucleotide
ccaaatgcccaaggctttac

SEQ ID NO: 2 - DNA, 20 bp, Artificial Sequence
  Synthetic oligonucleotide
ctgcaccgaattctcttccg

SEQ ID NO: 3 - DNA, 23 bp, Artificial Sequence
  Synthetic oligonucleotide
ccacatacacaaacttctcttag

SEQ ID NO: 4 - DNA, 20 bp, Artificial Sequence
  Synthetic oligonucleotide
tcacccttgaccacactgaa

SEQ ID NO: 5 - DNA, 20 bp, Artificial Sequence
  Synthetic oligonucleotide
gtgaggctgatatccccaga

SEQ ID NO: 6 - DNA, 20 bp, Artificial Sequence
  Synthetic oligonucleotide
gccaggcctgtaaactctga

SEQ ID NO: 7 - DNA, 20 bp, Artificial Sequence
  Synthetic oligonucleotide
tgggttaaaaatggcctctg

SEQ ID NO: 8 - DNA, 20 bp, Artificial Sequence
  Synthetic oligonucleotide
ttccgaaccattctttccag

SEQ ID NO: 9 - DNA, 20 bp, Artificial Sequence
  Synthetic oligonucleotide
gctgccttcttttgcagagt

SEQ ID NO: 10 - DNA, 20 bp, Artificial Sequence
  Synthetic oligonucleotide
gagaccctggagatggttga

SEQ ID NO: 11 - DNA, 20 bp, Artificial Sequence
  Synthetic oligonucleotide
gtgtgcttgtgtgcatgtga

SEQ ID NO: 12 - DNA, 20 bp, Artificial Sequence
  Synthetic oligonucleotide
cgtcagtcaagtcggcaata

SEQ ID NO: 13 - DNA, 20 bp, Artificial Sequence
  Synthetic oligonucleotide
caaattggagcgaaagggta

SEQ ID NO: 14 - DNA, 20 bp, Artificial Sequence
  Synthetic oligonucleotide
gagagctggacccgaatcta

SEQ ID NO: 15 - DNA, 20 bp, Artificial Sequence
  Synthetic oligonucleotide
ccggagccataaagaatctg

SEQ ID NO: 16 - DNA, 20 bp, Artificial Sequence
  Synthetic oligonucleotide
gtcaacttgccccaaaccta

SEQ ID NO: 17 - DNA, 20 bp, Artificial Sequence
  Synthetic oligonucleotide
gtggagtagaccctcccaca

SEQ ID NO: 18 - DNA, 20 bp, Artificial Sequence
  Synthetic oligonucleotide
tcgggattgtcacgtaatga

SEQ ID NO: 19 - DNA, 20 bp, Artificial Sequence
  Synthetic oligonucleotide
ctccggtaccgtcttttcag

SEQ ID NO: 20 - DNA, 20 bp, Artificial Sequence
  Synthetic oligonucleotide
gtggaagcagggtcaaaatc

SEQ ID NO: 21 - DNA, 20 bp, Artificial Sequence
  Synthetic oligonucleotide
gccatcagtaaagggtgcat

SEQ ID NO: 22 - DNA, 20 bp, Artificial Sequence
  Synthetic oligonucleotide
tgaccctatccacgatcaga

SEQ ID NO: 23 - DNA, 20 bp, Artificial Sequence
  Synthetic oligonucleotide
tcctgagtaagccagaagca

SEQ ID NO: 24 - DNA, 20 bp, Artificial Sequence
  Synthetic oligonucleotide
tgccaaggagtcgaggatgt

SEQ ID NO: 25 - DNA, 17 bp, Artificial Sequence
  Synthetic oligonucleotide
tcggcaccaggaaccaa

SEQ ID NO: 26 - DNA, 20 bp, Artificial Sequence
  Synthetic oligonucleotide
cgcaagcccttcagtgacat

SEQ ID NO: 27 - DNA, 21 bp, Artificial Sequence
  Synthetic oligonucleotide
cgcattgaacttgacagcaaa

SEQ ID NO: 28 - DNA, 18 bp, Artificial Sequence
  Synthetic oligonucleotide
tgcgggatgatggagaca

SEQ ID NO: 29 - DNA, 20 bp, Artificial Sequence
  Synthetic oligonucleotide
gcgaaagcgtcacaggtgta

SEQ ID NO: 30 - DNA, 23 bp, Artificial Sequence
  Synthetic oligonucleotide
tcgaaatctctccagtgacatga

SEQ ID NO: 31 - DNA, 24 bp, Artificial Sequence
  Synthetic oligonucleotide
ctacaatctcaccgaacacctcaa

SEQ ID NO: 32 - DNA, 19 bp, Artificial Sequence
  Synthetic oligonucleotide
caccttcccgctggacact

SEQ ID NO: 33 - DNA, 19 bp, Artificial Sequence
  Synthetic oligonucleotide
cctggccttcaccttggat

SEQ ID NO: 34 - DNA, 20 bp, Artificial Sequence
  Synthetic oligonucleotide
gccccttcacctctttagca

SEQ ID NO: 35 - DNA, 22 bp, Artificial Sequence
  Synthetic oligonucleotide
ccaagcactgggaaggtctaac

SEQ ID NO: 36 - DNA, 19 bp, Artificial Sequence
  Synthetic oligonucleotide
tggatctgtgcagcggatt

SEQ ID NO: 37 - DNA, 23 bp, Artificial Sequence
  Synthetic oligonucleotide
gggtcaccatagagctgaagaca

SEQ ID NO: 38 - DNA, 21 bp, Artificial Sequence
  Synthetic oligonucleotide
ggcaaaatactgggcatctga

SEQ ID NO: 39 - DNA, 20 bp, Artificial Sequence
  Synthetic oligonucleotide
ctccgtggagttgcacacat

SEQ ID NO: 40 - DNA, 18 bp, Artificial Sequence
  Synthetic oligonucleotide
accttgccagggcctgat

SEQ ID NO: 41 - DNA, 18 bp, Artificial Sequence
  Synthetic oligonucleotide
tggcctggtcaccggtaa

SEQ ID NO: 42 - DNA, 20 bp, Artificial Sequence
  Synthetic oligonucleotide
ggtgatggcgaagagacaca

SEQ ID NO: 43 - DNA, 20 bp, Artificial Sequence
  Synthetic oligonucleotide
gcttcctggatgaggcattc

SEQ ID NO: 44 - DNA, 26 bp, Artificial Sequence
  Synthetic oligonucleotide
ccactcaaacaagtttcatacacatt

SEQ ID NO: 45 - DNA, 21 bp, Artificial Sequence
  Synthetic oligonucleotide
ggatgtgacccttggctctgt

SEQ ID NO: 46 - DNA, 16 bp, Artificial Sequence
  Synthetic oligonucleotide
cgctgccgcgattctg

SEQ ID NO: 47 - DNA, 20 bp, Artificial Sequence
  Synthetic oligonucleotide
tttggactgcaggccgtact

SEQ ID NO: 48 - DNA, 21 bp, Artificial Sequence
  Synthetic oligonucleotide
ggatggtgatgcatcttttgg

SEQ ID NO: 49 - DNA, 19 bp, Artificial Sequence
  Synthetic oligonucleotide
atggcccgtgtggatcttt

SEQ ID NO: 50 - DNA, 24 bp, Artificial Sequence
  Synthetic oligonucleotide
ccatgagatcatctacacgatgct

SEQ ID NO: 51 - DNA, 22 bp, Artificial Sequence
  Synthetic oligonucleotide
ccctctgagatgaggactccat

SEQ ID NO: 52 - DNA, 25 bp, Artificial Sequence
  Synthetic oligonucleotide
tgctatggtctcagtgtcatctctt

SEQ ID NO: 53 - DNA, 21 bp, Artificial Sequence
  Synthetic oligonucleotide
ctctcaagggcattccatacg

SEQ ID NO: 54 - DNA, 22 bp, Artificial Sequence
  Synthetic oligonucleotide
cccagacatctcggttctcact

SEQ ID NO: 55 - DNA, 17 bp, Artificial Sequence
  Synthetic oligonucleotide
gccctgtgcccgagttt

SEQ ID NO: 56 - DNA, 16 bp, Artificial Sequence
  Synthetic oligonucleotide
tggcaccgggcacttg

SEQ ID NO: 57 - DNA, 20 bp, Artificial Sequence
  Synthetic oligonucleotide
caaaggctgcaaacggaaaa

SEQ ID NO: 58 - DNA, 23 bp, Artificial Sequence
  Synthetic oligonucleotide
ggttccacagacaaagtggtgat

SEQ ID NO: 59 - DNA, 24 bp, Artificial Sequence
  Synthetic oligonucleotide
ctctgactgagcggcattaaaatt

SEQ ID NO: 60 - DNA, 23 bp, Artificial Sequence
  Synthetic oligonucleotide
tgaaagacttgctcgagatgtca

SEQ ID NO: 61 - DNA, 20 bp, Artificial Sequence
  Synthetic oligonucleotide
cacacagagggccacaatgt

SEQ ID NO: 62 - DNA, 22 bp, Artificial Sequence
  Synthetic oligonucleotide
gccagctcagaacactggtcta

SEQ ID NO: 63 - DNA, 19 bp, Artificial Sequence
  Synthetic oligonucleotide
atgcccaaagcctggaaga

SEQ ID NO: 64 - DNA, 22 bp, Artificial Sequence
  Synthetic oligonucleotide
ggagtacgtcctgctgaaagct

SEQ ID NO: 65 - DNA, 22 bp, Artificial Sequence
  Synthetic oligonucleotide
cagcatcttcaatgtgcacaga

SEQ ID NO: 66 - DNA, 22 bp, Artificial Sequence
  Synthetic oligonucleotide
tcgggactcagtgcaagtacag

SEQ ID NO: 67 - DNA, 23 bp, Artificial Sequence
  Synthetic oligonucleotide
acagaggagagcctgaagatcct

SEQ ID NO: 68 - DNA, 21 bp, Artificial Sequence
  Synthetic oligonucleotide
ggctctggtccggtcttttag

SEQ ID NO: 69 - DNA, 21 bp, Artificial Sequence
  Synthetic oligonucleotide
ttctctgccacacggttttct

SEQ ID NO: 70 - DNA, 20 bp, Artificial Sequence
  Synthetic oligonucleotide
tgtgggcctttcagcttcat

SEQ ID NO: 71 - DNA, 21 bp, Artificial Sequence
  Synthetic oligonucleotide
cccagacctccaggtttcatt

SEQ ID NO: 72 - DNA, 19 bp, Artificial Sequence
  Synthetic oligonucleotide
gccgtcggagcacttactg

SEQ ID NO: 73 - DNA, 22 bp, Artificial Sequence
  Synthetic oligonucleotide
ggtacatgctcacagggatctg

SEQ ID NO: 74 - DNA, 23 bp, Artificial Sequence
  Synthetic oligonucleotide
gatcaggcgacatgtttaacgtt

SEQ ID NO: 75 - DNA, 21 bp, Artificial Sequence
  Synthetic oligonucleotide
agagcccagtcaaaccctttc

SEQ ID NO: 76 - DNA, 21 bp, Artificial Sequence
  Synthetic oligonucleotide
gcaggtgtcactagccttaat

(19) United States
(12) Patent Application Publication
(10) Pub. No.: US 2011/0138488 A1
(43) Pub. Date: Jun. 9, 2011

(54) DEFICIENCY IN THE HISTONE DEMETHYLASE JHDM2A RESULTS IN IMPAIRED ENERGY EXPENDITURE AND OBESITY

(76) Inventors: Yi Zhang, Chapel Hill, NC (US); Yuki Okada, Carrboro, NC (US); Keisuke Tatelshi, Tokyo (JP)

(21) Appl. No.: 12/999,059

(22) PCT Filed: Jun. 23, 2009
(86) PCT No.: PCT/US09/03733

§ 371 Date: Feb. 28, 2011

Related U.S. Application Data
(60) Provisional application No. 61/075,576, filed on Jun. 25, 2008.
(60) Provisional application No. 61/149,174, filed on Feb. 2, 2009.

(51) Int. Cl. G01N 33/00; A01K 67/00; C12N 5/10; C12Q 1/02; C12Q 1/68

(52) U.S. Cl. 800/3; 800/9; 435/354; 435/29; 435/6.11

(57) ABSTRACT

The present invention relates to animal models of metabolic disorders such as obesity, diabetes, metabolic syndrome, insulin resistance, hyperinsulinemia, glucose intolerance, hyperlipidemia, and the like. In particular, the invention relates to transgenic non-human animals having a reduction in functional Jhdm2a gene expression and use of such animals and cells having reduced Jhdm2a expression in drug discovery. The invention further relates to the identification of subjects having or at increased risk for developing a metabolic disorder based on a genetic marker in the Jhdm2a gene.

### RELATED APPLICATION INFORMATION

This application claims the benefit of U.S. Provisional Application No. 61/075,576; Filed Jun. 25, 2008, and U.S. Provisional Application No. 61/149,174 filed Feb. 2, 2009, the disclosure of which is incorporated by reference herein in its entirety.

### FIELD OF THE INVENTION

The present invention relates to animal models of metabolic disorders such as obesity, diabetes, metabolic syndrome, insulin resistance, hyperinsulinemia, glucose intolerance, hyperlipidemia, and the like. In particular, the invention relates to transgenic non-human animals having a reduction in functional Jhdm2a gene expression and use of such animals as well as cells having reduced Jhdm2a expression in drug discovery. The invention further relates to the identification of subjects having or at increased risk for developing a metabolic disorder based on a genetic marker in the Jhdm2a gene.

### BACKGROUND OF THE INVENTION

Histone methylation plays important roles in regulating diverse biological processes through controlling gene expression (Martin et al., Nat Rev Mol Cell Biol 6, 838 (November 2005). Recent studies indicate that the methylation state of histones can be dynamically regulated by histone methyltransferases and demethylases (Klose et al., Nat Rev Mol Cell Biol 8, 307 (2007)). The H3K9me1/2-specific demethylase JHDM2A/KDM3A plays an important role in nuclear hormone receptor mediated gene activation and male germ cell development (Okada et al., Nature 450, 119 (2007); Yamane et al., Cell 125, 483 (2006)).

In humans, excessive body fat deposition is closely linked to systemic insulin resistance, and diet-induced obesity is a primary risk factor for Type 2 diabetes. There is a need in the art for improved compositions and methods for treating and preventing metabolic disorders such as obesity, diabetes, metabolic syndrome and hyperlipidemia and methods of identifying those subjects that have or are at risk of developing such metabolic disorders.

### SUMMARY OF THE INVENTION

The invention is based, in part, on the discovery that genetically modified non-human animals (e.g., avians, non-human mammals) having a reduction in functional Jhdm2a gene expression develop a phenotype that resembles metabolic disorders in humans such as obesity, diabetes, metabolic syndrome, insulin resistance, hyperinsulinemia, glucose intolerance, hyperlipidemia, and the like. Thus, as one aspect, the invention provides a genetically modified non-human animal having a reduction in functional Jhdm2a gene expression. As a further aspect, the invention provides methods of using the non-human animals of the invention in drug discovery to identify compounds for treating and/or preventing metabolic disorders. As still a further aspect, the invention provides methods for identifying subjects having or at increased risk for developing a metabolic disorder based on a genetic marker in the Jhdm2a gene.

Accordingly, as one aspect, the invention provides a transgenic non-human animal (e.g., mouse) whose genome comprises a homozygous disruption of the Jhdm2a gene, wherein the disruption results in a deficiency in functional Jhdm2a gene expression in the animal, and wherein the animal exhibits one or more of the following characteristics relative to a wild-type animal: (i) obesity, (ii) systemic insulin resistance, (iii) hyperinsulinemia, (iv) hyperlipidemia, (v) impaired energy expenditure, and (vi) reduced β-adrenergic receptor stimulated O₂ consumption.

Also provided are isolated transgenic cells from the transgenic non-human animals of the invention and cell cultures comprising or produced by culturing the same.

As a further aspect, the invention provides a transgenic non-human animal (e.g., mouse) whose genome comprises a heterozygous disruption in the Jhdm2a gene, wherein the disruption results in a deficiency in functional Jhdm2a gene expression in the animal, and wherein the animal exhibits obesity on a high fat diet relative to a wild-type mouse. In particular embodiments, the animal is obese.

As another aspect, the invention provides a method of identifying a candidate compound for treating obesity, diabetes and/or metabolic syndrome, the method comprising:

administering a compound to an obese transgenic non-human animal (e.g., mouse) of the invention after the onset of obesity; and

determining the body weight and/or level of obesity in the transgenic animal,

wherein a reduction in body weight and/or obesity in the transgenic non-human animal administered the compound as compared with an untreated control animal indicates that the compound is a candidate for treating obesity, diabetes and/or metabolic syndrome.

As still a further aspect, the invention provides a method of identifying a candidate compound for preventing obesity, diabetes and/or metabolic syndrome, the method comprising:

administering a compound to a transgenic non-human animal (e.g., mouse) of the invention prior to the onset of obesity and for a time sufficient for the onset of obesity in an untreated control animal; and

determining the body weight and/or degree of obesity in the transgenic animal administered the compound,

wherein a reduction in body weight and/or obesity in the transgenic animal administered the compound relative to an untreated control animal indicates that the compound is a candidate for preventing obesity, diabetes and/or metabolic syndrome.

As yet a further aspect, the invention provides a method of identifying a candidate compound for treating obesity, diabetes and/or metabolic syndrome, the method comprising:

administering a compound to a transgenic non-human animal (e.g., mouse) of the invention after the onset of impaired energy expenditure; and

determining energy expenditure in the transgenic animal,

wherein an increase in energy expenditure in the transgenic animal administered the compound as compared with an untreated control animal indicates that the compound is a candidate for treating obesity, diabetes and/or metabolic syndrome.

As another aspect, the invention provides a method of identifying a candidate compound for preventing obesity, diabetes and/or metabolic syndrome, the method comprising:

administering a compound to a transgenic non-human animal (e.g., mouse) of the invention prior to the onset of impaired energy expenditure and for a time sufficient for the onset of impairments in energy expenditure in an untreated control animal; and

determining energy expenditure in the transgenic animal,

wherein an increase in energy expenditure in the transgenic animal administered the compound as compared with an untreated control animal indicates that the compound is a candidate for preventing obesity, diabetes and/or metabolic syndrome.

As a further aspect, the invention provides a method of identifying a candidate compound for treating obesity, diabetes and/or metabolic syndrome, the method comprising:

administering a compound to a transgenic non-human animal of the invention after the onset of reduced β-adrenergic receptor stimulated O₂ consumption; and

determining β-adrenergic receptor stimulated O₂ consumption by the transgenic animal,

wherein an increase in β-adrenergic receptor stimulated O₂ consumption in the transgenic animal administered the compound as compared with an untreated control animal indicates that the compound is a candidate for treating obesity, diabetes and/or metabolic syndrome.

As still a further aspect, the invention provides a method of identifying a candidate compound for preventing obesity, diabetes and/or metabolic syndrome, the method comprising:

administering a compound to a transgenic non-human animal (e.g., mouse) of the invention prior to the onset of reduced β-adrenergic receptor stimulated O₂ consumption and for a time sufficient for the onset of reduced β-adrenergic receptor stimulated O₂ consumption in an untreated control animal; and

determining β-adrenergic receptor stimulated O₂ consumption by the transgenic animal,

wherein an increase in β-adrenergic receptor stimulated O₂ consumption in the transgenic animal administered the compound relative to an untreated control animal indicates that the compound is a candidate for preventing obesity, diabetes and/or metabolic syndrome.

Still further, the invention provides a method of identifying a candidate compound for preventing and/or treating obesity, diabetes and/or metabolic syndrome, the method comprising:

contacting a cell (e.g., a genetically modified cell) having reduced expression of Jhdm2a with a compound,

determining energy expenditure and/or the activity of the AMPK-PGC-1α axis in the cell,

wherein increased energy expenditure and/or activity of the AMPK-PGC-1α axis in the cell contacted with the compound relative to an untreated control cell indicates that the compound is a candidate for preventing and/or treating obesity, diabetes and/or metabolic syndrome.

As yet another aspect, the invention provides a method of identifying a candidate compound for preventing and/or treating obesity, diabetes and/or metabolic syndrome, the method comprising:

contacting a cell (e.g., a genetically modified cell) having reduced expression of Jhdm2a with a compound,

determining PPARα signaling pathway activity and/or β-adrenergic receptor signaling pathway activity in the cell,

wherein an increase in PPARα signaling pathway activity and/or an increase in β-adrenergic receptor signaling pathway activity in the cell contacted with the compound relative to an untreated control cell indicates that the compound is a candidate for preventing and/or treating obesity, diabetes and/or metabolic syndrome.

The invention also provides methods of identifying a mammalian subject at increased risk for developing obesity, diabetes and/or metabolic syndrome, the method comprising detecting in the subject the presence of a genetic marker in the Jhmda2a gene, wherein the genetic marker is correlated with an increased risk of developing obesity, diabetes and/or metabolic syndrome, thereby identifying a mammalian subject having increased risk of developing obesity, diabetes and/or metabolic syndrome.

Also provided is a method of identifying a mammalian subject at increased risk for developing obesity, diabetes and/or metabolic syndrome, the method comprising:

(a) correlating the presence of a genetic marker in the Jhdm2a gene with an increased risk of developing obesity, diabetes and/or metabolic syndrome; and

(b) detecting the presence of the genetic marker of (a) in a mammalian subject, thereby identifying a mammalian subject at increased risk for developing obesity, diabetes and/or metabolic syndrome.

As yet another aspect, the invention provides a method of identifying a genetic marker in the Jhdma2 gene correlated with increased risk of a mammalian subject developing obesity, diabetes and/or metabolic syndrome, the method comprising:

(a) identifying a mammalian subject that has developed obesity, diabetes and/or metabolic syndrome;

(b) detecting in the mammalian subject the presence of a genetic marker in the Jhdm2a gene; and

(c) correlating the presence of the genetic marker of step (b) with the development of obesity, diabetes and/or metabolic syndrome, thereby identifying a genetic marker in the Jhdm2a gene correlated with increased risk of a mammalian subject developing obesity, diabetes and/or metabolic syndrome.

Also provided is a method of identifying a genetic marker in the Jhdma2 gene correlated with increased risk of a mammalian subject developing obesity, diabetes and/or metabolic syndrome, the method comprising:

(a) identifying a mammalian subject that has developed obesity, diabetes and/or metabolic syndrome;

(b) determining the nucleotide sequence of the Jhdm2a gene of the mammalian subject of (a);

(c) comparing the nucleotide sequence of (b) with the wild-type nucleotide sequence of the Jhdm2a gene;

(d) detecting a genetic marker in the nucleotide sequence of (b); and

(e) correlating the presence of the genetic marker of step (b) with the development of obesity, diabetes and/or metabolic syndrome in the mammalian subject of (a).

These and other aspects of the invention are described in more detail in the following description of the invention.

### BRIEF DESCRIPTION OF THE DRAWINGS

FIG. 1A shows the genomic structure of mouse Jhdm2a and the location of the β-Geo insertion in the YHA186 clone. V1 and V2 are two different variants (NM_{—}173001 and NM_{—}001038695, respectively).

FIG. 1B shows a diagram of the wild-type and mutant alleles. Boxes with numbers refer to the respective exons. The location of the probe used for Southern blot hybridization and sites of restriction enzyme digestion are shown. EV, EcoRV; Sp, Sph I.

FIG. 2 shows a diagram of the wild-type and mutant protein alleles. The mutant allele creates a fusion protein of JHDM2A (1-506)-β-Gal.

FIG. 3 shows quantitative RT-PCR analysis of the expression of the 3′ end of Jhdm2a. D7: Total RNA isolated from wild-type day 7 mouse testis. The expression level in adult Jhdm2a^{+/+} was arbitrarily defined as 1. Data presented are means±s.d. from three mice for each genotype.

FIG. 4A shows diagrams of the wild-type and mutant alleles before and after Cre recombination. Cre-mediated recombination results in a mutant allele that lacks exons 22-24 (encompassing the JmjC domain). a, b and c indicate primers for genotyping.

FIG. 4B shows PCR genotyping of targeted alleles.

FIG. 4C shows western blot analysis, demonstrating successful knockout of Jhdm2a protein in skeletal muscle tissue.

FIG. 5A shows Jhdm2a-deficient mice exhibiting an obesity phenotype. Shown is a representative photograph of 7-month old littermates of Jhdm2a^{+/+} and Jhdm2a^{-/-} mice.

FIG. 5B shows a growth curve of male Jhdm2a^{+/+ (n=}10) and Jhdm2a^{-/-} (n=17) mice indicating that loss of JHDM2A function leads to obese phenotype as the mice age.

FIG. 5C shows a growth curve of female Jhdm2a^{+/+} (n=10) and Jhdm2a^{-/-} (n=24) mice indicating that loss of JHDM2A function leads to obese phenotype as the mice age.

FIG. 6A shows an image of 12-month old Jhdm2a^{+/+} (left) and Jhdm2a^{G/G} (right) mice.

FIG. 6B shows that Jhdm2a^{G/G} mice become obese. Average body weights of 4-month-old Jhdm2a^{+/+} and Jhdm2a^{G/G} littermates. (n=5). *p<0.05. **p<0.01.

FIG. 7 shows quantitative RT-PCR analysis of Jhdm2a mRNA levels in different mouse organs. Primers specific for HPRT mRNA were used for normalization. Jhdm2a mRNA is expressed at a relatively higher level in the oxidative organs such as brown adipose tissue (BAT) and skeletal muscles. The highest expression is detected in testis. VWAT, visceral white adipose tissue; HT, hypothalamus.

FIG. 8 shows Jhdm2a-deficiency results in body fat accumulation, as measured by Magnetic Resonance Imaging (16-week littermates, n=6/group). **p<0.05. N.S., not significant.

FIG. 9 shows the average weight of inguinal white adipose tissue (IWAT) in 3-month old mice. n=6. *p<0.05.

FIG. 10 shows enlarged white adipose cells in Jhdm2a^{-/-} mice. HE staining of white adipose tissues derived from adult (7-month old) littermate Jhdm2a^{-/+} or Jhdm2a^{-/-} mice.

FIG. 11 shows Jhdm2a-deficiency results in hyperlipidemia as indicated by the increased levels of serum-free fatty acid (FFA), triglyceride (TG) and total cholesterol (TCHO) in Jhdm2a^{+/+} (n=16) compared with that in Jhdm2a^{-/-} (n=10) mice. *p<0.05. **p<0.01.

FIG. 12 shows a growth curve of littermates fed with a high fat diet (HFD) or normal chow. Solid lines, high fat diet group; dashed lines, normal chow group. BW, body weight. n=4 per group. *p<0.03. Data are mean and s.e.m.

FIG. 13 shows Jhdm2a-deficiency results in glucose-intolerance. Glucose tolerance test (GTT) was performed using 12-week old littermates. n=5 per group. *p<0.05.

FIG. 14 shows Jhdm2a-deficiency results in decreased insulin sensitivity. Insulin tolerance test (ITT) was performed using 12-week old littermates. n=5 per group. *p<0.05.

FIG. 15 shows Jhdm2a-deficiency results in increased plasma insulin levels (12-week old littermates, n=3/group). *p<0.05.

FIG. 16A shows Jhdm2a deficiency does not affect food intake in 6-week-old Jhdm2a KO mice.

FIG. 16B shows Jhdm2a deficiency does not affect food intake in 16-week-old Jhdm2a KO mice, regardless of the fat content in the diet.

FIG. 17 shows Jhdm2a deficiency does not significantly alter serum leptin levels in 6-week-old Jhdm2a KO mice.

FIG. 18A shows Jhdm2a deficiency results in decreased O₂ consumption. Whole body O₂ consumption in 12-16 week old mice was measured by calorimetry. Shown is the average O₂ consumption value over 3 days and 3 nights. n=6 per group. *p<0.05. **p<0.01.

FIG. 18B shows Jhdm2a deficiency results in decreased heat generation. Whole body heat generation in 12-16 week old mice was measured by calorimetry. Shown is the average heat value generated over 3 days and 3 nights. n=6 per group. *p<0.05. **p<0.01.

FIG. 19 shows Jhdm2a deficiency results in decreased physical activity. Physical activity was measured using 12-16 week old littermates. Shown is the average activity during the night period. n=6 per group. *p<0.05.

FIG. 20 shows Jhdm2a deficiency does not significantly alter plasma hormone concentrations. C.S., corticosterone (n=4); N.E., norepinephrine (n=6), T3, triiodothyronine (n=6). N.S., not significant.

FIG. 21A shows gene ontology enrichment analysis, wherein Jhdm2a knock-out results in the down-regulation of a significant proportion of genes involved in several metabolic processes. Genes were down-regulated at least two-fold in knock-out muscle versus wild-type muscle.

FIG. 21B shows a pie chart representing step-wise hierarchical tree drill down of significantly enriched terms under the heading Primary Metabolic Process. Enrichment p values <0.05 are indicated in parentheses.

FIG. 21C shows a pie chart representing step-wise hierarchical tree drill down of significantly enriched terms under the heading Primary Metabolic Process (p=0.0343). Enrichment p values <0.05 are indicated in parentheses.

FIG. 21D shows the Primary Metabolic Process sub-category. Enrichment p values <0.05 are indicated in parentheses.

FIG. 21E shows the Cellular Metabolic Process sub-category. Enrichment p values <0.05 are indicated in parentheses.

FIG. 22 shows the effects of Jhdm2a deficiency on PPAR signaling pathway in muscle. AFFYMETRIX microarray fold change data from RNA samples corresponding to Jhdm2a KO muscle vs. wild-type muscle was overlaid onto the PPAR signaling pathway using GenMAPP2 software. Entities are indicated by expression fold change in KO vs. wild-type as indicated.

FIG. 23 shows Jhdm2a deficiency results in decreased expression of genes involved in PPAR signaling in the Jhdm2a knock-out soleus muscle. **p<0.05.

FIG. 24A shows Jhdm2a deficiency results in decreased β-oxidation in primary muscle cells. Jhdm2a^{+/+} and Jhdm2a^{-/-} primary muscle cells were isolated and incubated with ¹⁴C-palmitic acid. Released ¹⁴CO₂ was measured by scintillation counting. n=5 per group. **P<0.01.

FIG. 24B shows Jhdm2a deficiency results in decreased glycerol release in isolated soleus muscle. n=5 per group; **P<0.01.

FIG. 25 shows Jhdm2a deficiency impaired AICAR-induced respiration increase in primary myotubes. Jhdm2a^{+/+} and Jhdm2a^{-/-} primary myotubes were treated with 500 μM AICAR for 16 hours. Rates of oxygen consumption were normalized to cell counts. *p<0.05.

FIG. 26 shows Jhdm2a directly binds to the PPRE of the PPARα enhancer. Soleus muscles were used for ChIP followed by qRT-PCR. Results are normalized to IgG. *P<0.05.

FIG. 27 shows Jhdm2a deficiency results in increased H3K9me2 levels within the PPARα enhancer. The signal in wild-type cells is set as 1. *P<0.05.

FIG. 28 shows cultured Jhdm2a knock-out myocytes exhibit decreased PPARα expression, which can be partially restored by overexpression of the JHDM2A gene. Wild-type primary cultured myocytes served as a control for qRT-PCR. *P<0.05.

FIG. 29 shows ChIP analysis in primary cultured myocytes. Jhdm2a deficiency causes an increase in H3K9me2 levels at the PPRE of the PPARα enhancer. This change can be partially rescued by overexpression of Jhdm2a. The signal in wild-type cells was set as 1. *P<0.05. Data are mean and s.e.m.

FIG. 30A shows BAT from chow-fed Jhdm2a KO mice is significantly enlarged and slightly pale when compared with that derived from their wild-type littermates.

FIG. 30B shows Jhdm2a deficiency induces fat accumulation in intrascapular BAT.

FIG. 31 shows Jhdm2a deficiency impairs the ability of mice to maintain body temperature when exposed to cold. Shown is the body temperature of 12-week-old mice at different times after exposure to cold temperature (4° C.). n=5 per group. *P<0.05.

FIG. 32A shows Jhdm2a deficiency impairs ISO-induced oxygen consumption in BAT. *P<0.05.

FIG. 32B shows Jhdm2a deficiency results in decreased glycerol release in BAT. *P<0.05.

FIG. 33 shows qRT-PCR analysis demonstrating decreased expression of genes involved in mitochondrial function in the Jhdm2a knock-out BAT. *P<0.05.

FIG. 34 shows Jhdm2a deficiency impairs the cold-induced activation of Ucp1, but not Dio2, in BAT. N>4 per group. The mRNA level was normalized by 3684 mRNA, and the relative quantity in Jhdm2a^{+/+} BAT at 20° C. was defined as 1. **P<0.01. Data are mean and s.e.m.

FIG. 35 shows Jhdm2a deficiency does not affect the expression of factors responsible for Ucp1 upregulation. mRNA levels were analyzed before (20° C.) and after cold exposure (4° C.) in brown fat cells utilized for the ChIP experiments.

FIG. 36A shows that Jhdm2a knock-down (KD) in HIB1B cells impairs ISO-induced Ucp1 transcriptional activation. Ctrl, control. **P<0.01.

FIG. 36B shows defective ISO-induced Ucp1 activation caused by Jhdm2a knock-down in HIB1B cells can be partially restored by JHDM2A overexpression. *P<0.05.

FIG. 37 shows qRT-PCR (top panel) and western blot analysis (bottom panels) demonstrating that Jhdm2a is upregulated by ISO in HIB1B cells. **P<0.01.

FIG. 38 shows that Jhdm2a deficiency does not affect the mRNA expression of factors responsible for Ucp1 upregulation.

FIG. 39 shows ChIP analysis demonstrating that Jhdm2a directly binds to PPRE (amplicon A) of the Ucp1 enhancer after β-adrenergic stimulation.

FIG. 40 shows ChIP analysis demonstrating that H3K9me2 at the Ucp1 gene enhancer is demethylated by Jhdm2a after ISO treatment. The Jhdm2a knock-down-induced increase in H3K9me2 level could be partially rescued by overexpression of JHDM2A coupled with ISO treatment. *P<0.05.

FIG. 41 shows ChIP followed by qPCR indicating Jhdm2a knock-down impairs β-adrenergic receptor activation-stimulated PPARγ and Rxrα recruitment to the PPRE of the Ucp1 gene enhancer. Results are normalized to IgG, and are shown as the fold-enrichment relative to that in the control cells without ISO. *P<0.05.

FIG. 42 shows ChIP followed by qPCR indicating Jhdm2a knock-down impairs β-adrenergic receptor activation-stimulated recruitment of PPARγ and Rxrα co-activators Pgc1α, p300 and Src1 to the PPRE of the Ucp1 gene enhancer. Results are normalized to IgG, and are shown as fold of enrichment relative to that in the control cells without ISO. *P<0.05. Data are mean and s.e.m.

FIG. 43 shows that Jhdm2a deficiency does not affect the protein expression of factors responsible for Ucp1 upregulation. Protein level was analyzed using the HIB1B cell lysates used for the ChIP experiments.

FIG. 44 shows decreased oxygen consumption in Jhdm2a KO mice. Loss of the Jhdm2a function results in decreased oxygen consumption during night cycle in 6-week-old mice.

FIG. 45 shows Jhdm2a knock-down in C2C12 myotubes causes decreased expression of PGC-1α target genes. *p<0.05.

FIG. 46 shows Jhdm2a knock-down in C2C12 myotubes results in decreased β-oxidation and O₂ consumption. *p<0.05.

FIG. 47 shows Jhdm2a knock-down in C2C12 myotubes impairs the AMPK-induced PGC-1α upregulation, which can be rescued by expression of a wild-type but not by a catalytic mutant JHDM2A. Relative expression level of PGC-1 [symbol] in Ctrl cells without AICAR treatment is defined as 1. *p<0.05.

FIG. 48 shows AMPK-induced H3K9 demethylation in the PGC-1α promoter is dependent on JHDM2A. Wild-type and Jhdm2a knock-down C2C12 myotubes with or without AICAR treatment were used for ChIP followed by qPCR. Antibodies against H3K9me1 and H3K9me2 were used in the ChIP assays. Results were normalized to input, and are shown as fold of enrichment relative to that in the wild-type C2C12 cells without AICAR treatment. *p<0.05.

### DETAILED DESCRIPTION OF THE INVENTION

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless the context indicates otherwise, it is specifically intended that the various features of the invention described herein can be used in any combination.

Any feature of the invention that is specifically described herein can be included or omitted from the invention (e.g., can be disclaimed).

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

As used herein the term “consisting essentially of” (and grammatical variations) means that the composition, article, product or method does not comprise any elements that materially change the functioning of the composition, article, product or method other than those elements specifically recited.

The invention provides transgenic non-human animals that have been genetically modified to reduce functional Jhdm2a gene expression. Animals of the present invention are, in general, non-human mammals and/or avians including but not limited to monkeys, chimps, dogs, cats, sheep, goats, cattle, rabbits, pigs, rodents (e.g., hamsters, gerbils, rats, guinea pigs and/or mice), chickens, turkeys, ducks and/or quail. In some embodiments, the non-human animal is a laboratory animal (e.g., monkey, rodent, dog, pig, bird, etc.). In some embodiments, the animal is a non-human primate, a species of domestic livestock (e.g., horse, cattle, sheep, pig, goat, chicken and the like) and/or a companion animal (e.g., cat, dog, guinea pig, gerbil, hamster, and the like).

Animals may be male and/or female and may be of any age, including neonatal, immature, adult and/or senescent animals. In particular embodiments, the animal is an immature animal (e.g., before sexual maturity; sexual maturity is at about four to eight weeks in the mouse). In embodiments of the invention, the animal is an adult animal (e.g., after sexual maturity), optionally after the onset of metabolic defects in energy expenditure and/or obesity due to the reduction in functional Jhmd2a gene expression.

A “metabolic disorder” as used herein includes but is not limited to any disorder or condition associated with an impairment in energy metabolism including but not limited to obesity, diabetes (e.g., type II/non-insulin dependent diabetes), metabolic syndrome, insulin resistance, hyperinsulinemia, glucose intolerance, hyperglycemia (e.g., fasting hyperglycemia), hyperlipidemia, and the like.

In general, the non-human animals of the invention deposit excess fat as compared with wild-type animals (e.g., wild-type littermates) after reaching sexual maturity. For example, a transgenic mouse having reduced functional Jhmd2a expression may begin to deposit excess fat as compared with wild-type littermates at about 5 to 6 months of age, and will generally be clearly obese by 7 months, and fat deposition will peak at about one year of age. Metabolic impairments in energy expenditure (e.g., reductions in basal metabolic rate, physical activity, and/or adaptive thermogenesis) will generally precede the development of obesity in the animal.

A “genetically modified,” “recombinant” or “transgenic” non-human animal (and like terms) of the invention comprises modifications to its genome such that the animal has a reduction in functional Jhdm2a gene expression. The genomic modification can be present in the somatic and/or germ cells of the animal, optionally, in all of the somatic cells and/or all of the germ cells of the animal.

“Congenic” strains may be created, which are useful for generating non-human animals (e.g., mice) that are nearly identical except for a selected genotype/phenotype (see, e.g., U.S. Pat. No. 7,202,393 to Matsushima). Congenic animals can be generated by mating two genetically distinct inbred strains and then backcrossing the descendants with one of the parental or ancestral strains (the “recipient” strain), e.g., for two generations, followed by inbreeding sister and brother, with or without selecting for particular markers or phenotypes. Using this method, the recipient on average contributes the greater proportion of the genome to each congenic strain. Backcrossing generally increases homozygosity twice as fast as sibling mating. Other methods of creating congenic strains may also be used, and alternative methods may be used, as will be appreciated by those of skill in the art. For example, the number of backcrosses may vary, resulting in different genomic proportions from the recipient. Selection for the genotype/phenotype of interest may also be performed at certain steps as desired.

In representative embodiments, the non-human animal of the invention is obese. Typically, the animal exhibits the onset of obesity in adulthood (e.g., after reaching sexual maturity). For example, in mice, the onset of obesity may be at about 1, 2, 3, 4 or 5 months. Generally, obesity is understood to be an increase in body mass of 20% or more over the ideal body mass. In particular embodiments, the animal has an increase in body mass of at least about 20%, 30%, 40%, 50%, 60%, 70%, 80%, 90%, 100%, 150%, 200% or more and/or an increase in the mass and/or proportion of body fat by weight of at least about 20%, 30%, 40%, 50%, 60%, 70%, 80%, 90%, 100%, 150%, 200% or more relative to a suitable control (e.g., a wild-type animal of the same species, strain, general age and/or gender, for example, a wild-type littermate). Methods of determining normal body weight are known in the art. For example, in humans, normal body weight can be defined as a BMI index of 18.5-24.9 kg/meter² (NHLBI (National Heart Lung and Blood Institute) Obesity Education Initiative. The Practical Guide-Identification, Evaluation and Treatment of Overweight and Obesity in Adults. NIH Publication No. 00-4084 (2000); obtainable at http://www.nhlbi.nih.gov/guidelines/obesity/prctad_b.pdf). In particular embodiments, the non-human animal has a BMI index of about 24.9 kg/meter² or greater.

In exemplary embodiments, the non-human animal exhibits obesity when fed a high fat diet (e.g., adult-onset obesity on a high fat diet). The term “high fat diet” is understood in the art and includes but is not limited to diets in which at least about 50%, 60%, 70% or more of the calories are derived from fat. In a representative example of this embodiment, the non-human animal is heterozygous for the genetic modification resulting in a reduction in functional Jhdm2a gene expression, e.g., an obese heterozygous animal.

Obesity can be determined by any suitable method known in the art, such as by determining body weight, BMI, mass of body fat, body fat as a percentage of body weight, and the like.

In embodiments of the invention, the animal exhibits glucose intolerance, hyperglycemia (e.g., fasting hyperglycemia), insulin resistance, hyperinsulinemia and/or hyperlipidemia relative to a wild-type animal. In embodiments of the invention, the onset of these characteristics is in the adult animal (e.g., after sexual maturity). Hyperglycemia is characterized by excessive blood (or plasma) glucose levels. Methods of diagnosing and evaluating hyperglycemia are known in the art. In general, fasting hyperglycemia is characterized by blood or plasma glucose concentration above the normal range after a subject has fasted for at least eight hours (e.g., the normal range is about 70-120 mg/dL). Postprandial hyperglycemia is generally characterized by blood or plasma glucose concentration above the normal range one to two hours after food intake by a subject.

By “insulin resistance” or “insulin insensitivity” it is meant a state in which a given level of insulin produces a less than normal biological effect (e.g., uptake of glucose). Insulin resistance is particularly prevalent in obese individuals or those with type-2 diabetes or metabolic syndrome. In type-2 diabetics, the pancreas is generally able to produce insulin, but there is an impairment in insulin action. As a result, hyperinsulinemia is commonly observed in insulin-resistant subjects. Insulin resistance is less common in type-I diabetics; although in some subjects, higher dosages of insulin have to be administered over time indicating the development of insulin resistance/insensitivity. The term “insulin resistance” or “insulin insensitivity” refers to systemic insulin resistance/insensitivity unless specifically indicated otherwise. Methods of evaluating insulin resistance/insensitivity are known in the art, for example, hyperinsulinemic/euglycemic clamp studies, insulin tolerance tests, uptake of labeled glucose and/or incorporation into glycogen in response to insulin stimulation, and measurement of known components of the insulin signaling pathway.

“Glucose intolerance” is characterized by an impaired ability to maintain blood (or plasma) glucose concentrations following a glucose load (e.g., by ingestion or infusion) resulting in hyperglycemia. Glucose intolerance is generally indicative of an insulin deficiency or insulin resistance. Methods of evaluating glucose tolerance/intolerance are known in the art, e.g., the oral glucose tolerance test.

“Hyperlipidemia” can include elevations in serum free fatty acids, triglycerides, LDL, HDL, VLDL and/or total cholesterol. Methods of measuring serum lipids are standard in the art.

In representative embodiments, the non-human transgenic animals of the invention display metabolic impairments in energy metabolism and/or impairments in the AMPK-PGC-1α axis.

Accordingly, in some embodiments, the animal has a reduction in energy expenditure (e.g., basal metabolic rate, physical activity and/or adaptive thermogenesis) relative to a wild-type animal. Methods of determining basal metabolic rate are known in the art, for example, by measuring oxygen consumption and/or heat generation by the animal. Methods of evaluating physical activity are known in the art, e.g., by measuring horizontal and/or vertical movement. Methods of measuring adaptive thermogenesis are also known in the art, for example, the ability of the animal to maintain body weight in response to cold stress.

In representative embodiments, there are defects in brown adipose tissue function and/or cold intolerance in the non-human animals of the invention relative to a wild-type animal.

In embodiments of the invention, there is a reduction in cold stress induced PPARα, UCP1 and/or PGC-1α upregulation in the animal (e.g., in brown adipose tissue, white adipose tissue, liver, skeletal muscle and/or testis) relative to the level of induction observed in a wild-type animal.

In embodiments of the invention, expression of PGC-1α and/or its downstream targets (including without limitation MCAD, ATP5J, UCP2, COX7A and other downstream targets of PGC-1α), PPARα, and/or PGC-1β is reduced in the animal (e.g., brown adipose tissue, white adipose tissue, liver, skeletal muscle and/or testis) relative to a wild-type animal.

As used herein, the terms “express,” “expressing,” or “expression” (or grammatical variants thereof) in reference to a gene or coding sequence can refer to transcription to produce an RNA and, optionally translation to produce a functional protein or peptide. Thus, the terms “express,” “expressing,” “expression” and the like can refer to events at the transcriptional, post-transcriptional, translational and/or post-translational level.

In embodiments of the invention, serum leptin levels are increased and/or there is increased AMPK phosphorylation in the animal (e.g., in brown adipose tissue, white adipose tissue, liver, skeletal muscle and/or testis) relative to a wild-type animal. According to this embodiment of the invention, the animal can be an adult animal (e.g., after sexual maturity) after the onset of excess fat accumulation and is optionally an obese animal.

In embodiments of the invention, there is a reduction in AICAR-induced AMPK activation of cellular respiration (e.g., in brown adipose tissue, white adipose tissue, liver, skeletal muscle and/or testis) by the non-human animals of the invention relative to a wild-type animal.

In embodiments of the invention, there is a reduction in AMPK-mediated PGC-1α induction (e.g., in brown adipose tissue, white adipose tissue, liver, skeletal muscle and/or testis) in the non-human animals of the invention relative to a wild-type animal.

In embodiments of the invention, there is a reduction in β-oxidation (e.g., in brown adipose tissue, white adipose tissue, liver, skeletal muscle and/or testis) in the non-human animals of the invention relative to a wild-type animal.

In embodiments of the invention, there is a reduction in mono- and/or di-methylation at lysine 9 of histone H3 (H3K9me1 and H3K9me2) in response to AICAR induced H3K9 demethylation at the PGC-1α promoter (e.g., in brown adipose tissue, white adipose tissue, liver, skeletal muscle and/or testis) in the non-human animals of the invention relative to a wild-type animal.

In other representative embodiments of the invention, there is an impairment in the PPARα signaling pathway (e.g., in brown adipose tissue, white adipose tissue, liver, skeletal muscle and/or testis) in the non-human animals of the invention relative to a wild-type animal. As non-limiting examples, there can be impaired expression and/or activity of PPARα and/or one or more of its downstream targets (including without limitation UCP2, MCAD, LCAD, VLCAD, AQP7).

In representative embodiments of the invention, H3K9me2 levels at the PPAR responsive element (PPRE) of the PPARα gene are increased (e.g., in brown adipose tissue, white adipose tissue, liver, skeletal muscle and/or testis) in the non-human animals of the invention relative to a wild-type animal.

In representative embodiments, there is an impairment in β-adrenergic signaling pathways (e.g., in brown adipose tissue, white adipose tissue, liver, skeletal muscle and/or testis) in the non-human animals of the invention relative to a wild-type animal. As a non-limiting illustration, β-adrenergic receptor stimulated (e.g., in the presence of β-adrenergic receptor agonists such as isoproterenol) O₂ consumption and/or glycerol release is reduced (e.g., in brown adipose tissue, white adipose tissue, liver, skeletal muscle and/or testis) in the non-human animals of the invention relative to a wild-type animal. As a further example, β-adrenergic receptor stimulated (e.g., in the presence of β-adrenergic receptor agonists such as isoproterenol) expression and/or activity of one or more downstream targets involved in mitochondrial function (including without limitation PPARα, UCP3, CPT2 and/or LCAD) is reduced (e.g., in brown adipose tissue, white adipose tissue, liver, skeletal muscle and/or testis) in the non-human animals of the invention relative to a wild-type animal.

In representative embodiments of the invention, β-adrenergic receptor mediated reductions in H3K9me2 levels at the Ucp1 enhancer region are reduced (e.g., in brown adipose tissue, white adipose tissue, liver, skeletal muscle and/or testis) in the non-human animals of the invention relative to a wild-type animal.

In representative embodiments of the invention, β-adrenergic receptor mediated increase in recruitment of transcription factors and/or co-activators to the Ucp1 gene is reduced (e.g., in brown adipose tissue, white adipose tissue, liver, skeletal muscle and/or testis) in the non-human animals of the invention relative to a wild-type animal. For example, β-adrenergic receptor mediated increase in PPARγ, RXRα, PGC1α, CBP/p300 and/or SRC1 binding to the UCP1 enhancer is reduced (e.g., in brown adipose tissue, white adipose tissue, liver, skeletal muscle and/or testis) in the non-human animals of the invention relative to a wild-type animal.

In reference to the metabolic impairments and dysregulation of gene expression discussed herein, the terms “reduce,” “reduced,” “reducing” and the like refer to a decrease of at least about 20%, 30%, 40%, 50%, 60%, 70%, 80%, 85%, 90%, 95%, 98% or more in the relevant parameter.

In reference to the metabolic impairments and dysregulation of gene expression discussed herein, the terms “increase,” “increases,” “increasing” and like terms refer to an elevation of at least about 20%, 30%, 40%, 50%, 60%, 70%, 80%, 90%, 100%, 150%, 200% or more in the relevant parameter.

In embodiments of the invention, the impairments in energy expenditure, gene regulation and/or plasma leptin concentrations described herein commence in the adult animal (e.g., after the animal reaches sexual maturity). Alternatively, in embodiments of the invention, the impairments in energy expenditure, gene regulation, and plasma leptin concentrations described herein commence in the immature animal (e.g., prior to sexual maturation, for example, an animal that is younger than about 8, 7, 6, 5, 4, 3, 2 or 1 week(s) of age).

In some embodiments, the animal does not have a significant change in plasma corticosterone, triiodothyronine (T3) and/or norepinephrine as compared with a suitable control.

The transgenic non-human animals of the invention have been genetically modified to result in a reduction in functional Jhdm2a gene expression. “Functional Jhdm2a gene expression” and like terms refers to expression at the mRNA and/or protein level, e.g., transcription to produce an mRNA encoding a functional JHDM2A protein (for example, having H3K9 demethylase activity). Thus, the genetic modification can result in an impairment at the transcriptional, post-transcriptional, translational and/or post-translational level and/or can produce a protein that has substantially reduced or no biologic activity (e.g., histone demethylase activity).

A variety of methods are known in the art for reducing functional gene expression including methods for producing “knockout,” “knockin,” and “knockdown” animals, all of which may be used to produce the transgenic non-human animals of the present invention. Thus, in some embodiments, the transgenic non-human animal comprises a heterozygous and/or homozygous disruption in the Jhdm2a gene, e.g., as a knockout or knockin mutation. The disruption can encompass any portion of the gene that results in reduced functional Jhdm2A protein including the 5′ untranslated region, one or more exons and/or one or more introns. In particular embodiments, the disruption comprises the catalytic JmjC domain. For example, in a transgenic mouse of the invention, the disruption can comprise the catalytic JmjC domain in exons 22, 23 and 24, and can optionally comprise all of exons 22, 23 and 24. In other embodiments, the disruption can comprise a “knockin” upstream of the JmjC catalytic domain.

In particular embodiments, the non-human animal of the invention is homozygous for the genetic modification (e.g., disruption to the Jhdm2a gene) that results in reduced functional Jhdm2a gene expression. According to this embodiment, production of functional JHDM2a protein can be reduced by 75%, 85%, 90%, 95%, 99% or more relative to a suitable control in one or more, or even all, tissues.

In other embodiments, the non-human animal of the invention is heterozygous for the genetic modification that results in reduced functional Jhdm2a gene expression. According to this embodiment, production of functional JHDM2a protein can be reduced about one-half the level observed in homozygous animals (e.g., by about 40% or 45% to about 50%, 55% or 60% or about 50% to about 55% or 60%).

In exemplary embodiments, the invention provides a transgenic non-human animal (e.g., mouse) whose genome comprises a homozygous disruption of the Jhdm2a gene, wherein the disruption results in a deficiency in functional Jhdm2a gene expression in the animal, and wherein the animal exhibits one or more of the following characteristics relative to a wild-type animal: (i) obesity, (ii) systemic insulin resistance, (iii) hyperinsulinemia, (iv) hyperlipidemia, (v) reduction in energy expenditure, (vi) reduction in cold stress induced PPARα, UCP1 and/or PGC-1α upregulation; (vii) reduction in expression of PGC-1α and/or its down-stream targets (including UCP2, MCAD, ATP5J, COX7A), PPARα and/or PGC-16; (viii) increase in leptin concentrations in blood, plasma or serum; (ix) increased AMPK phosphorylation; (x) reduction in AICAR-induced AMPK activation of cellular respiration; (xi) reduction in AMPK-mediated PGC-1α induction; (xii) reduction in β-oxidation; (xiii) reduction in AICAR induced H3K9me1 and H3K9me2 demethylation at the PGC-1α promoter; (xiv) reduction in PPARα signaling pathway activity (described herein); (xv) increase in H3K9me2 levels at the PPRE of the PPARα gene; (xvi) reduction in β-adrenergic receptor signaling pathway activity (described herein); and/or (xvii) reduction in β-adrenergic receptor mediated H3K9 demethylation at the Ucp1 enhancer region. In particular embodiments, one or more of the foregoing characteristics exhibits a pattern of adult-onset.

“Wild-type” gene sequences of a given species are those DNA or protein sequences that are most highly conserved within or across species and/or which are generally accepted as the wild-type gene in the art. For example, the genomic sequences of the mouse and human Jhdm2a genes can be found at GenBank Accession Nos. NC_{—}000072.5. and NC_{—}000002.10, respectively.

As used here, a “wild type” non-human animal is one that does not contain the genetic manipulations of the invention to reduce functional Jhdm2a gene expression. In representative embodiments, the genome of the wild-type non-human animal is otherwise significantly or substantially identical to the transgenic and/or congenic non-human animal of the invention (e.g., littermates).

A “knockout” of a target gene means an alteration in a host cell genome that results in reduced expression of the target gene, e.g., by introduction of a mutation (e.g., insertion) into a coding or noncoding region of the target gene, which mutation reduces expression of the target gene. Mammals containing a knockout of Jhdm2a may be heterozygous or homozygous with respect to the mutation that causes the knockout.

The term “conditional knockout” as used herein refers to an animal in which the knocked out gene is selectively or preferentially knocked out in a particular tissue (e.g., in brown adipose tissue, white adipose tissue, skeletal muscle, testis and/or liver) and/or at a particular time of development. Such conditional knockout animals can be produced by a variety of techniques, such as with site-specific recombinases such as Cre/lox (to create “floxed” mice or mice having a “floxed” gene) or Tnpl/TRT (see, e.g., U.S. Pat. No. 7,083,976), and/or with a tetracycline-controllable transactivator (see, e.g., U.S. Pat. Nos. 6,783,757 and 6,252,136).

“Floxed” mice or “Cre/lox conditional knockout” animals are known. The Cre recombinase catalyzes recombination between 34 by loxP recognition sequences (Sauer, B. and Henderson, N., Proc. Natl. Acad. Sci. USA 85:5166-5170, 1988). The loxP sequences can be inserted into the genome of embryonic stem cells by homologous recombination such that they flank one or more exons of a gene of interest (making a “floxed” gene). Animals homozygous for the floxed gene are generated from these embryonic stem cells by conventional techniques and are crossed to a second animal that harbors a Cre transgene under the control of a tissue type- or cell type-specific transcriptional promoter. In progeny that are homozygous for the floxed gene and that carry the Cre transgene, the floxed gene will be deleted by Cre/loxP recombination, but only in those cell types in which the Cre gene-associated promoter is active. See U.S. Pat. No. 6,583,333; see also U.S. Pat. No. 6,946,244.

The recombinase enzyme can be provided by any suitable method. For example, purified recombinase enzyme can be provided by direct microinjection. In another embodiment, recombinase is expressed from a co-transfected construct or vector in which the recombinase gene is operably linked to a functional promoter. Further, tissue-specific or inducible recombinase constructs can be employed which allow the choice of when and where recombination occurs. One method for practicing the inducible forms of recombinase-mediated recombination involves the use of vectors that use inducible or tissue-specific promoters or other gene regulatory elements to express the desired recombinase activity. The inducible expression elements can optionally be positioned to allow the inducible control or activation of expression of the desired recombinase activity. Examples of such inducible promoters or other gene regulatory elements include, but are not limited to, tetracycline, metallothionein, ecdysone, and other steroid-responsive promoters, rapamycin responsive promoters, and the like (No, et al. Proc. Natl. Acad. Sci. USA, 93:3346-51 (1996); Furth, et al. Proc. Natl. Acad. Sci. USA, 91:9302-6 (1994)). Additional control elements that can be used include promoters requiring specific transcription factors such as viral promoters. Vectors incorporating such promoters only express recombinase activity in cells that express the necessary transcription factors. In particular embodiments, the promoters or regulatory elements is preferentially or specifically active or inducible in brown adipose tissue, white adipose tissue, liver, skeletal muscle and/or testis.

Another example of a conditional knockout can be made using FRT-Flpase technology, also well know to those skilled in the art of gene-targeting mice.

A large number of suitable alternative site-specific recombinases have been described, and their genes can be used to produce the transgenic non-human animals of the invention. Such recombinases include the Int recombinase of bacteriophage lambda (with or without Xis) (Weisberg, R. et. al., in Lambda II, (Hendrix, R., et al., Eds.), Cold Spring Harbor Press, Cold Spring Harbor, N.Y., pp. 211-50 (1983)); Tpnl and the β-lactamase transposons (Mercier, et al., J. Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J. Bacteriol., 172:610-18 (1990)); the B. subtilis SpoIVC recombinase (Sato, et al., J. Bacteriol. 172:1092-98 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol., 205:493-500 (1989)). Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al. (Mol Cell. Biochem., 92:107-16 (1990)); and Cregg & Madden (Mol. Gen. Genet., 219:320-23 (1989)).

Other transgenic non-human animals of the invention can comprise any mutation that impairs functional Jhdm2a gene expression, by reducing transcription, translation and/or post-translational processing and the like and/or by producing a JHDM2A protein with substantially reduced or no detectable histone demethylase activity. Any nucleotide in a 5′ regulatory region (e.g., the promoter), an exonic region and/or an intron region of the Jhdm2a gene can be mutated or deleted in order to produce a mouse that is defective for functional Jhdm2a gene expression. Such mice can be generated with a targeting construct containing a site-specific mutation, or as a “knock-in”, in which a mutated sequence is targeted to an acceptor site, such as one generated in a conventional gene-targeting strategy.

An “RNAi knockdown” of a target gene means an alteration in the animal's genome that results in altered expression of the target gene, e.g., by introduction of a expression cassette that encodes an RNAi (e.g., shRNA or siRNA) that binds to the target gene or its transcripts to decrease expression thereof. Mammals containing a knockdown of Jhdm2a may be heterozygous or homozygous with respect to the insert that expresses the sequence responsible for expressing RNAi targeted against Jhdm2a. See, e.g., D. Pawitt et al., RNAi-knock-down mice: an emerging technology for post-genomic functional genetics, Cytogenet. Genome Res. 105 (2-4): 412-21 (2004).

A “knock-in” of a target gene generally refers to the replacement of endogenous genetic material (e.g., a gene or a portion of a gene) with exogenous genetic material. The transgenic non-human animal may be heterozygous or homozygous with respect to the mutation (e.g., insertion) that causes the knock-in.

The production of transgenic animals, including “knockout,” “knockin,” and “knockdown” animals, is known and can be carried out in accordance with known techniques or variations thereof which will be apparent to those skilled in the art, for example, U.S. Pat. No. 7,115,795 to Forsberg et al. (University of Guelph), U.S. Pat. No. 6,888,047 to Wu et al. (New York University), and U.S. Pat. No. 6,872,868 to Wagner et al. (Ohio University) all disclose transgenic non-human mammals derived through microinjection of DNA into the pronucleus or cytoplasm of embryos at the single cell stage. See also U.S. Pat. No. 5,175,385 to Wagner et al. and U.S. Pat. No. 5,175,384 to Krimpenfort et al. More specifically, “knockin” and “knockout” transgenic rats derived through microinjection of fertilized ova are disclosed in U.S. Pat. No. 7,262,336 to Young et al. (Wyeth) and U.S. Pat. No. 6,372,956 to Goldsmith et al. (The J. David Gladstone Institutes) “Knockin” and “knockout” cows are also known: U.S. Pat. No. 7,074,983 to Robl et al.

See also U.S. Pat. No. 7,022,893 to Takeda et al. and U.S. Pat. No. 6,218,595 to Giros et al., as well as U.S. Pat. No. 6,344,596 to W. Velander et al. (American Red Cross); U.S. Pat. No. 6,339,183 to T. T. Sun (New York University); U.S. Pat. No. 6,331,658 to D. Cooper and E. Koren; U.S. Pat. No. 6,255,554 to H. Lubon et al. (American National Red Cross; Virginia Polytechnic Institute); U.S. Pat. No. 6,204,431 to P. Prieto et al. (Abbott Laboratories); U.S. Pat. No. 6,166,288 to L. Diamond et al. (Nextran Inc., Princeton, N.J.); U.S. Pat. No. 5,959,171 to J. M. Hyttinin et al. (Pharming BV); U.S. Pat. No. 5,880,327 to H. Lubon et al. (American Red Cross); U.S. Pat. No. 5,639,457 to G. Brem; U.S. Pat. No. 5,639,940 to I. Garner et al. (Pharmaceutical Proteins Ltd.; Zymogenetics Inc); U.S. Pat. No. 5,589,604 to W. Drohan et al. (American Red Cross); U.S. Pat. No. 5,602,306 to Townes et al. (UAB Research Foundation); U.S. Pat. No. 4,736,866 to Leder and Stewart (Harvard); and U.S. Pat. No. 4,873,316 to Meade and Lonberg (Biogen).

The non-human animals of the present invention include progeny and their descendents of first generation animals produced by the methods described herein. Such animals, including congenic animals, can be produced in accordance with known techniques, including but not limited to those described in U.S. Pat. No. 6,465,714. In general, progeny can be created by (a) providing a first (mate or female) recombinant parent animal produced as described above, and a second parent animal, wherein at least the first parent exhibits a phenotype of the invention; and then (b) crossing the first and second parent animal with one another to produce a progeny animal that exhibits that phenotype. Subsequent generations can be further produced in accordance with known techniques.

The invention also encompasses methods of identifying candidate compounds for preventing and/or treating obesity, diabetes, metabolic syndrome, hyperlipidemia, insulin resistance, hyperinsulinemia, hyperglycemia and/or glucose intolerance using the non-human animals of the invention, cells isolated therefrom, cell cultures comprising such cells or cell cultures produced by culturing such cells.

Thus, in representative embodiments, the invention provides a method of identifying a candidate compound for treating obesity, diabetes, metabolic syndrome, hyperlipidemia, insulin resistance, hyperinsulinemia, hyperglycemia and/or glucose intolerance, the method comprising: administering a compound to an obese non-human transgenic animal of the invention (e.g., an adult animal, for example, an animal that is at least about 2, 3, 4, 5, 6, 7, 8 months of age or older); and determining the body weight and/or level of obesity in the animal, wherein a reduction in body weight and/or obesity in the animal administered the compound as compared with an untreated control animal indicates that the compound is a candidate for treating obesity, diabetes, metabolic syndrome, hyperlipidemia, insulin resistance, hyperinsulinemia, hyperglycemia and/or glucose intolerance.

Generally, the compound is administered for a time sufficient for a reduction in body weight and/or level of obesity to be observed if the compound is effective (e.g., at least about 3 days, 1 week, 2 weeks, 3 weeks, four weeks, 8 weeks or longer).

The invention also provides a method of identifying a candidate compound for preventing obesity, diabetes, metabolic syndrome, hyperlipidemia, insulin resistance, hyperinsulinemia, hyperglycemia and/or glucose intolerance, the method comprising: administering a compound to a non-human transgenic animal of the invention prior to the onset of obesity (e.g., to an immature animal prior to sexual maturation, for example, an animal that is younger than about 8, 7, 6, 5, 4, 3, 2 or 1 week(s) of age) and for a time sufficient for the onset of obesity in an untreated control animal; and determining the body weight and/or degree of obesity in the non-human transgenic animal administered the compound, wherein a reduction in body weight and/or obesity in the transgenic non-human animal administered the compound relative to an untreated control animal indicates that the compound is a candidate for preventing obesity, diabetes, metabolic syndrome, hyperlipidemia, insulin resistance, hyperinsulinemia, hyperglycemia and/or glucose intolerance.

Methods of determining body weight and obesity are known in the art, for example, as described herein.

As a further aspect, the invention provides a method of identifying a candidate compound for treating obesity, diabetes, metabolic syndrome, hyperlipidemia, insulin resistance, hyperinsulinemia, hyperglycemia and/or glucose intolerance, the method comprising: administering a compound to an obese non-human transgenic animal of the invention (e.g., an adult animal, for example, an animal that is at least about 2, 3, 4, 5, 6, 7, 8 months of age or older); and determining energy expenditure in the animal, wherein an increase in energy expenditure in the animal administered the compound as compared with an untreated control animal indicates that the compound is a candidate for treating obesity, diabetes, metabolic syndrome, hyperlipidemia, insulin resistance, hyperinsulinemia, hyperglycemia and/or glucose intolerance.

Generally, the compound is administered for a time sufficient for an increase in energy expenditure to be observed if the compound is effective (e.g., at least about 3 days, 1 week, 2 weeks, 3 weeks, four weeks, 8 weeks or longer).

The invention further provides a method of identifying a candidate compound for preventing obesity, diabetes, metabolic syndrome, hyperlipidemia, insulin resistance, hyperinsulinemia, hyperglycemia and/or glucose intolerance, the method comprising: administering a compound to a transgenic non-human animal of the invention prior to the onset of impaired energy expenditure (e.g., to an immature animal prior to sexual maturation, for example, an animal that is younger than about 8, 7, 6, 5, 4, 3, 2 or 1 week(s) of age) and for a time sufficient for the onset of impairments in energy expenditure in an untreated control animal; and determining energy expenditure in the transgenic non-human animal administered the compound, wherein an increase in energy expenditure in the transgenic non-human animal administered the compound as compared with an untreated control animal indicates that the compound is a candidate for preventing obesity, diabetes, metabolic syndrome, hyperlipidemia, insulin resistance, hyperinsulinemia, hyperglycemia and/or glucose intolerance.

Methods of determining energy expenditure and impairments in energy expenditure are routine in the art and include without limitation evaluating basal metabolic rate, physical activity and/or adaptive thermogenesis.

In other representative embodiments, the invention provides a method of identifying a candidate compound for treating obesity, diabetes, metabolic syndrome, hyperlipidemia, insulin resistance, hyperinsulinemia, hyperglycemia and/or glucose intolerance, the method comprising: administering a compound to a non-human transgenic animal of the invention; and determining β-adrenergic receptor stimulated (e.g., by administering in a β-adrenergic receptor agonist such as isoproterenol to the animal) O₂ consumption by the transgenic non-human animal, wherein an increase in β-adrenergic receptor stimulated O₂ consumption in the transgenic non-human animal administered the compound as compared with an untreated control animal indicates that the compound is a candidate for treating obesity, diabetes, metabolic syndrome, hyperlipidemia, insulin resistance, hyperinsulinemia, hyperglycemia and/or glucose intolerance. Optionally, this embodiment of the invention can be practiced with an immature animal prior to sexual maturation, for example, an animal that is younger than about 8, 7, 6, 5, 4, 3, 2 or 1 week(s) of age. Alternatively, in some instances, this embodiment can be practiced with an adult animal, for example, an animal that is at least 2, 3, 4, 5, 6, 7, 8 months of age or older, optionally an obese adult animal.

As used herein, an “untreated control” animal can be any suitable control including but not limited to a transgenic non-human animal that has not been given any treatment or has been treated with a placebo. The untreated control can optionally be the same animal prior to treatment. In representative embodiments, the untreated control is from the same species and strain and is matched for age and, optionally, gender.

Thus, in some embodiments, the methods of the invention comprise a “base-line” measurement prior to administering the compound and one or more follow-up measurements after administration of the compound to evaluate effectiveness.

The administering step may be carried out by any suitable technique depending upon the particular compound, including parenteral administration (e.g., intraperitoneal, intramuscular, intravenous, intra-arterial administration), subcutaneous administration oral administration, inhalation administration, transdermal administration, etc.

The invention also provides cells isolated from the transgenic non-human animals of the invention. The invention further contemplates cell cultures comprising the cells isolated from the non-human transgenic animal and/or cell cultures produced by culturing the isolated cells. The cells and cell cultures derived from the non-human transgenic animals of the invention can be used in the screening methods of the invention.

Such cells and cell cultures include somatic cells and cultures including but not limited to brown adipose tissue cells and cultures, white adipose tissue cells and culture, liver cells and culture, skeletal muscle cells and culture, testis cells and culture, brain cells and culture, pancreatic cells and culture, fibroblast cells and culture, epithelial cells and culture, endodermal cells and culture, smooth muscle cells and culture, cardiac myocytes and culture, and kidney cells and culture, and germ cells and culture, etc.

In particular embodiments, the cell or cell culture is from an obese animal. In embodiments, the cell or cell culture is from an animal that exhibits impairments in energy expenditure. In embodiments of the invention, the cell or cell culture is from an adult animal. In embodiments the cell or cell culture is from an immature animal (e.g., prior to sexual maturation, for example, an animal that is younger than about 8, 7, 6, 5, 4, 3, 2 or 1 week(s) of age). In embodiments of the invention, the cell or cell culture is from an animal prior to the onset of obesity. In embodiments of the invention, the cell or cell culture is from an animal prior to the onset of impairments in energy expenditure.

The invention further provides a method of identifying a candidate compound for preventing or treating obesity, diabetes, metabolic syndrome, hyperlipidemia, insulin resistance, hyperinsulinemia, hyperglycemia and/or glucose intolerance, the method comprising: contacting a cell comprising a heterozygous or homozygous disruption of the Jhdm2a gene (described herein) with a compound; and determining:

(i) energy expenditure (e.g., oxygen consumption and/or β-oxidation),

(ii) the activity of the AMPK-PGC-1α axis (including without limitation determining expression of PGC-1α and/or its downstream targets (e.g., UCP2, MCAD, ATP5J and/or COX7A, etc.),

(iii) AMPK-mediated PGC-1α induction,

(iv) JHDM2A enrichment at the PGC-1α promoter, and/or

(v) AICAR induced H3K9me1 and/or H3K9me2 demethylation at the PGC-1α promoter,

wherein partial or complete normalization of one or more these parameters (e.g., increase in energy expenditure, JHDM2a enrichment at the PGC-1α promoter, the activity of the AMPK-PGC-1α axis and/or AICAR induced H3K9me1 and/or H3K9me2 demethylation at the PGC-1α promoter) in the cell contacted with the compound relative to an untreated control cell indicates that the compound is a candidate for preventing and/or treating obesity, diabetes, metabolic syndrome, hyperlipidemia, insulin resistance, hyperinsulinemia, hyperglycemia and/or glucose intolerance.

In representative embodiments of the foregoing method, the cell is derived from a transgenic non-human animal of the invention (cells are as described herein).

The invention further provides a method of identifying a candidate compound for preventing or treating obesity, diabetes, metabolic syndrome, hyperlipidemia, insulin resistance, hyperinsulinemia, hyperglycemia and/or glucose intolerance, the method comprising: contacting a cell comprising a heterozygous or homozygous disruption of the Jhdm2a gene (described herein) with a compound; and determining:

(i) PPARα signaling pathway activity, for example, by determining expression and/or activity of PPARα and/or one or more of its downstream targets, including without limitation UCP2, MCAD, LCAD, VLCAD, AQP7;

(ii) H3K9me2 levels at the PPRE of the PPARα gene;

(iii) β-adrenergic receptor signaling pathway activity, for example, by determining β-adrenergic receptor stimulated O₂ consumption; glycerol release; expression and/or activity of one or more downstream targets involved in mitochondrial function (including without limitation PPARα, UCP3, CPT2 and/or LCAD); β-adrenergic receptor stimulated recruitment of transcription factors (including without limitation PPARγ, RXRα, PGC1α, CBP/p300 and/or SRC1) and/or co-activators to the Ucp1 gene; and/or β-adrenergic receptor stimulated upregulation of UCP1 expression and/or activity; and/or

(iv) β-adrenergic receptor mediated H3K9 demethylation at the Ucp1 enhancer region,

wherein partial or complete normalization of one or more of these parameters (e.g., increase in PPARα signaling pathway activity, β-adrenergic receptor signaling pathway activity and/or β-adrenergic receptor mediated H3K9 demethylation at the Ucp1 enhancer region and/or reduced H3K9me2 levels at the PPRE of the PPARα gene) in the cell contacted with the compound relative to an untreated control cell indicates that the compound is a candidate for preventing and/or treating obesity, diabetes, metabolic syndrome, hyperlipidemia, insulin resistance, hyperinsulinemia, hyperglycemia and/or glucose intolerance.

In representative embodiments, the cell is a cell derived from a transgenic non-human animal of the invention (cells are as described herein).

In particular embodiments, a nucleic acid comprising the appropriate regulatory region(s) (e.g., promoter and/or enhancer) of the PPARα gene operably associated with the coding sequence for a reporter protein (e.g., Green Fluorescent Protein, luciferase, β-galactosidase, alkaline phosphatase) is transiently and/or stably introduced into the cell or cell culture. The reporter protein can provide a simple read-out of PPARα expression and/or PPARα pathway activity.

Likewise, in other embodiments, a nucleic acid comprising the appropriate regulatory region(s) (e.g., promoter and/or enhancer) of the Ucp1 gene operably associated with the coding sequence for a reporter protein (e.g., Green Fluorescent Protein, luciferase, β-galactosidase, alkaline phosphatase) is transiently and/or stably introduced into the cell. The reporter protein can provide a simple read-out of UCP1 expression and/or β-adrenergic pathway activity.

In other representative embodiments, the invention further provides a method of identifying a candidate compound for preventing or treating obesity, diabetes, metabolic syndrome, hyperlipidemia, insulin resistance, hyperinsulinemia, hyperglycemia and/or glucose intolerance, the method comprising: contacting a cell comprising a heterozygous or homozygous disruption of the Jhdm2a gene (described herein) with a compound; and determining mitochondrial metabolism, wherein increased mitochondrial metabolism in the cell contacted with the compound relative to an untreated control cell indicates that the compound is a candidate for preventing and/or treating obesity, diabetes, metabolic syndrome, hyperlipidemia, insulin resistance, hyperinsulinemia, hyperglycemia and/or glucose intolerance.

In representative embodiments, the cell is derived from a transgenic non-human animal of the invention (cells are as described herein).

The screening methods of the invention can also be practiced with cells and cell cultures in which Jhdm2a expression is reduced, for example, using “knockdown” methods such as RNAi knockdown (e.g., using shRNA, siRNA, etc.), miRNA or antisense RNA approaches. Alternatively, the Jhdm2a gene can be disrupted. Methods of gene disruption are known in the art (for example, as described herein). Such cells and cell cultures include somatic cells and cultures including but not limited to brown adipose tissue cells and cultures, white adipose tissue cells and culture, liver cells and culture, skeletal muscle cells and culture, testis cells and culture, brain cells and culture, pancreatic cells and culture, fibroblast cells and culture, epithelial cells and culture, endodermal cells and culture, smooth muscle cells and culture, cardiac myocytes and culture, and kidney cells and culture, and germ cells and culture, etc. The cells and cell culture can further be embryonic cells and culture. The cell or cell culture can be contacted with the RNAi, miRNA or antisense RNA transiently by exogenously adding the RNAi to the cell or cell culture. Alternatively, the genome of the cell or cultured cells can be altered to express the RNAi, miRNA or antisense RNA molecule.

According to this embodiment, the cells can be derived from animals of any age including embryonic, neonatal, immature, senescent and adult animals and can further be from obese animals. The cells can also be derived from immortalized cell lines.

The invention also provides a method of identifying a candidate compound for preventing or treating obesity, diabetes, metabolic syndrome, hyperlipidemia, insulin resistance, hyperinsulinemia, hyperglycemia and/or glucose intolerance, the method comprising: contacting a Jhdm2a “knockdown” cell or cell culture with a compound; and determining:

(i) energy expenditure (e.g., oxygen consumption and/or β-oxidation),

(ii) the activity of the AMPK-PGC-1α axis (including without limitation determining expression of PGC-1α and/or its downstream targets (e.g., UCP2, MCAD, ATP5J and/or COX7A, etc.),

(iii) AMPK-mediated PGC-1α induction,

(iv) JHDM2A enrichment at the PGC-1α promoter, and/or

(v) AICAR H3K9me1 and/or H3K9me2 demethylation at the PGC-1α promoter,

wherein partial or complete normalization of one or more these parameters (e.g., increase in energy expenditure, JHDM2a enrichment at the PGC-1α promoter, the activity of the AMPK-PGC-1α axis and/or AICAR induced H3K9me1 and/or H3K9me2 demethylation at the PGC-1α promoter) in the cell or cell culture contacted with the compound relative to an untreated control cell or cell culture indicates that the compound is a candidate for preventing and/or treating obesity, diabetes, metabolic syndrome, hyperlipidemia, insulin resistance, hyperinsulinemia, hyperglycemia and/or glucose intolerance.

The invention also provides a method of identifying a candidate compound for preventing or treating obesity, diabetes, metabolic syndrome, hyperlipidemia, insulin resistance, hyperinsulinemia, hyperglycemia and/or glucose intolerance, the method comprising: contacting a Jhdm2a “knockdown” cell or cell culture with a compound; and determining:

(i) PPARα signaling pathway activity, for example, by determining expression and/or activity of PPARα and/or one or more of its downstream targets, including without limitation UCP2, MCAD, LCAD, VLCAD, AQP7;

(ii) H3K9me2 levels at the PPRE of the PPARα gene;

(iii) β-adrenergic receptor signaling pathway activity, for example, by determining β-adrenergic receptor stimulated O₂ consumption; glycerol release; expression and/or activity of one or more downstream targets involved in mitochondrial function (including without limitation PPARα, UCP3, CPT2 and/or LCAD); β-adrenergic receptor stimulated recruitment of transcription factors (including without limitation PPARγ, RXRα, PGC1α, CBP/p300 and/or SRC1) and/or co-activators to the Ucp1 gene; and/or β-adrenergic receptor stimulated upregulation of UCP1 expression and/or activity; and/or

(iv) β-adrenergic receptor mediated H3K9 demethylation at the Ucp1 enhancer region,

wherein partial or complete normalization of one or more of these parameters (e.g., increase in PPARα signaling pathway activity, β-adrenergic receptor signaling pathway activity and/or β-adrenergic receptor mediated H3K9 demethylation at the Ucp1 enhancer region and/or reduced H3K9me2 levels at the PPRE of the PPARα gene) in the cell or cell culture contacted with the compound relative to an untreated control cell or cell culture indicates that the compound is a candidate for preventing and/or treating obesity, diabetes, metabolic syndrome, hyperlipidemia, insulin resistance, hyperinsulinemia, hyperglycemia and/or glucose intolerance.

In particular embodiments, a nucleic acid comprising the appropriate regulatory region(s) (e.g., promoter and/or enhancer) of the PPARα gene operably associated with the coding sequence for a reporter protein (e.g., Green Fluorescent Protein, luciferase, β-galactosidase, alkaline phosphatase) is transiently and/or stably introduced into the cell or cell culture. The reporter protein can provide a simple read-out of PPARα expression and/or PPARα pathway activity.

Likewise, in other embodiments, a nucleic acid comprising the appropriate regulatory region(s) (e.g., promoter and/or enhancer) of the Ucp1 gene operably associated with the coding sequence for a reporter protein (e.g., Green Fluorescent Protein, luciferase, β-galactosidase, alkaline phosphatase) is transiently and/or stably introduced into the cell or cell culture. The reporter protein can provide a simple read-out of UCP1 expression and/or β-adrenergic pathway activity.

In other representative embodiments, the invention provides a method of identifying a candidate compound for preventing or treating obesity, diabetes, metabolic syndrome, hyperlipidemia, insulin resistance, hyperinsulinemia, hyperglycemia and/or glucose intolerance, the method comprising: contacting a Jhdm2a “knockdown” cell or cell culture with a compound; and determining mitochondrial metabolism, wherein increased mitochondrial metabolism in the cell or cell culture contacted with the compound relative to an untreated control cell or cell culture indicates that the compound is a candidate for preventing and/or treating obesity, diabetes, metabolic syndrome, hyperlipidemia, insulin resistance, hyperinsulinemia, hyperglycemia and/or glucose intolerance. Methods of measuring mitochondrial metabolism are known in the art (e.g., as described herein).

The foregoing embodiments have been described with respect to cells having a knockdown in Jhdm2a expression, but can also be practiced with any cell having a genetic modification that results in a reduction in Jhdm2a gene expression.

Those skilled in the art will appreciate that the inventive screening methods can be practiced with the animals and cells of the invention by detecting the activity of any suitable parameter (e.g., any of the molecular, biochemical, metabolic, physiological and/or phenotypic changes described herein) and evaluating the effect of a candidate compound on that parameter, for example, to determine if the compound can partially or completely normalize the affected parameter as compared with an appropriate control animal or cell.

In reference to the screening methods of the invention, the terms “reduce,” “reduced,” “reducing” and the like refer to a decrease of at least about 20%, 30%, 40%, 50%, 60%, 70%, 80%, 85%, 90%, 95%, 98% or more in the relevant parameter.

In reference to the screening methods of the invention, the terms “increase,” “increases,” “increasing” and like terms refer to an elevation of at least about 20%, 30%, 40%, 50%, 60%, 70%, 80%, 90%, 100%, 150% or more in the relevant parameter.

In particular embodiments, by the terms “normalize,” “normalized,” “normalizing” and the like, it is meant that a parameter that is reduced in the animals and cells of the invention is increased to at least about 60%, 70%, 75%, 80%, 85%, 90%, 95% or more of the value in a suitable control animal (e.g., a wild-type animal of the same species, strain, general age and/or gender, for example, a wild-type littermate). In particular embodiments, the terms “normalize,” “normalized,” “normalizing” and the like indicate that a parameter that is increased in the animals and cells of the invention is reduced to less than about 200%, 150%, 140%, 130%, 125%, 120%, 110% or less of the value in a suitable control animal. In representative embodiments, by the terms “normalize,” “normalized,” “normalizing” and the like, it is meant that the value is essentially the same as in a suitable control animal.

The transgenic non-human animals of the invention are phenotypically similar to humans with diabetes (e.g., type II diabetes), obesity and/or metabolic syndrome. Thus, genetic markers in the Jhdm2a gene can be used to identify subjects at increased risk for developing obesity, diabetes (e.g., type II diabetes), metabolic syndrome, hyperinsulinemia, insulin resistance, glucose intolerance, hyperglycemia and/or hyperlipidemia.

Accordingly, the invention provides a method of identifying a mammalian subject (e.g., a human) at increased risk for developing obesity, diabetes (e.g., type II diabetes), metabolic syndrome, hyperinsulinemia, insulin resistance, glucose intolerance, hyperglycemia and/or hyperlipidemia, the method comprising detecting in the subject the presence of a genetic marker in the Jhmda2a gene, wherein the genetic marker is correlated with an increased risk of developing obesity, diabetes (e.g., type II diabetes), metabolic syndrome, hyperinsulinemia, insulin resistance, glucose intolerance, hyperglycemia and/or hyperlipidemia, thereby identifying a mammalian subject having increased risk of developing obesity, diabetes (e.g., type II diabetes), metabolic syndrome, hyperinsulinemia, insulin resistance, glucose intolerance, hyperglycemia and/or hyperlipidemia.

The invention also provides a method of identifying a mammalian subject at increased risk for developing obesity, diabetes (e.g., type II diabetes), metabolic syndrome, hyperinsulinemia, insulin resistance, glucose intolerance, hyperglycemia and/or hyperlipidemia, the method comprising: (a) correlating the presence of a genetic marker in the Jhdm2a gene with an increased risk of developing obesity, diabetes (e.g., type II diabetes), metabolic syndrome, hyperinsulinemia, insulin resistance, glucose intolerance, hyperglycemia and/or hyperlipidemia; and (b) detecting the presence of the genetic marker of (a) in a mammalian subject, thereby identifying a mammalian subject at increased risk for developing obesity, diabetes (e.g., type II diabetes), metabolic syndrome, hyperinsulinemia, insulin resistance, glucose intolerance, hyperglycemia and/or hyperlipidemia.

The invention also provides a method of identifying a genetic marker in the Jhdma2 gene correlated with increased risk of a mammalian subject developing obesity, diabetes (e.g., type II diabetes), metabolic syndrome, hyperinsulinemia, insulin resistance, glucose intolerance, hyperglycemia and/or hyperlipidemia, the method comprising: (a) identifying a mammalian subject that has developed obesity, diabetes (e.g., type II diabetes), metabolic syndrome, hyperinsulinemia, insulin resistance, glucose intolerance, hyperglycemia and/or hyperlipidemia; (b) detecting in the mammalian subject the presence of a genetic marker in the Jhdm2a gene; and (c) correlating the presence of the genetic marker of step (b) with the development of obesity, diabetes (e.g., type II diabetes), metabolic syndrome, hyperinsulinemia, insulin resistance, glucose intolerance, hyperglycemia and/or hyperlipidemia, thereby identifying a genetic marker in the Jhdm2a gene correlated with increased risk of a mammalian subject developing obesity, diabetes (e.g., type II diabetes), metabolic syndrome, hyperinsulinemia, insulin resistance, glucose intolerance, hyperglycemia and/or hyperlipidemia.

As a further aspect the invention provides a method of identifying a genetic marker in the Jhdma2 gene correlated with increased risk of a mammalian subject developing obesity, diabetes (e.g., type II diabetes), metabolic syndrome, hyperinsulinemia, insulin resistance, glucose intolerance, hyperglycemia and/or hyperlipidemia, the method comprising: (a) identifying a mammalian subject that has developed obesity, diabetes (e.g., type II diabetes), metabolic syndrome, hyperinsulinemia, insulin resistance, glucose intolerance, hyperglycemia and/or hyperlipidemia; (b) determining the nucleotide sequence of the Jhdm2a gene of the mammalian subject of (a); (c) comparing the nucleotide sequence of (b) with the wild-type nucleotide sequence of the Jhdm2a gene (e.g., a “reference sequence”); (d) detecting a genetic marker in the nucleotide sequence of (b); and (e) correlating the presence of the genetic marker of step (b) with the development of obesity, diabetes (e.g., type II diabetes), metabolic syndrome, hyperinsulinemia, insulin resistance, glucose intolerance, hyperglycemia and/or hyperlipidemia in the mammalian subject of (a).

In particular embodiments, the methods of the invention further comprise performing a population based study to detect the polymorphisms in a group of subjects with obesity, diabetes (e.g., type II diabetes), metabolic syndrome, hyperinsulinemia, insulin resistance, glucose intolerance, hyperglycemia and/or hyperlipidemia and racially and/or ethnically matched controls.

The term “genetic marker” as used herein refers to a region of a nucleotide sequence (e.g., in a chromosome) that is subject to variability (i.e., the region can be polymorphic for a variety of alleles). For example, a single nucleotide polymorphism (SNP) in a nucleotide sequence is a genetic marker that is polymorphic for two alleles. Other examples of genetic markers of this invention can include but are not limited to microsatellites, restriction fragment length polymorphisms (RFLPs), repeats (i.e., duplications), insertions, deletions, and the like.

The mammalian subject can be any animal that is susceptible to obesity, diabetes (e.g., type II diabetes), metabolic syndrome, hyperinsulinemia, insulin resistance, glucose intolerance, hyperglycemia and/or hyperlipidemia. Examples of subjects of this invention can include, but are not limited to, humans, non-human primates, dogs, cats, horses, cattle, goats, pigs, sheep, guinea pigs, mice, rats and rabbits, as well as any other domestic, commercially or clinically valuable animal, including animal models.

Human subjects can be male and/or female and can be of any age including neonates, infants, juveniles, adults and/or senescent individuals.

In the methods described herein, the detection of a genetic marker in a subject can be carried out according to methods well known in the art. For example, nucleic acid (e.g., DNA or RNA) is obtained from any suitable sample from the subject that will contain nucleic acid and the nucleic acid is then prepared and analyzed according to well-established protocols for the presence of genetic markers according to the methods of this invention. In some embodiments, analysis of the nucleic acid can be carried out by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA)). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, or by electrophoresis. Thus, the present invention can further provide oligonucleotides for use as primers and/or probes for detecting and/or identifying genetic markers according to the methods of this invention.

The genetic markers of this invention can be correlated with obesity, diabetes (e.g., type II diabetes), metabolic syndrome, hyperinsulinemia, insulin resistance, glucose intolerance, hyperglycemia and/or hyperlipidemia according to methods well known in the art. In general, identifying such correlation involves conducting analyses that establish a statistically significant association and/or a statistically significant correlation between the presence of a genetic marker or a combination of markers and the phenotypic trait in the subject. An analysis that identifies a statistical association (e.g., a significant association) between the marker or combination of markers and the phenotype establishes a correlation between the presence of the marker or combination of markers in a subject and the particular phenotype being analyzed.

The correlation can involve one or more than one genetic marker (e.g., two, three, four, five, or more) in any combination. In some embodiments of this invention, one or more or all of the genetic markers are located in the JHDM2a gene.

The genetic markers can be used individually or in combination. Thus, in some embodiments, the methods of the invention can include correlations between various genetic markers located in the Jhdm2s gene and obesity, diabetes (e.g., type II diabetes), metabolic syndrome, hyperinsulinemia, insulin resistance, glucose intolerance, hyperglycemia and/or hyperlipidemia.

The genetic marker can be a single nucleotide polymorphism (SNP). Exemplary single nucleotide polymorphisms include but are not limited to T for G, T for A, C for A, C for T, A for G, A for C, A for T, G for A and G for T substitutions. SNPs within the human Jhdm2a gene are known (see FIG. 31), one or more of which may be used in the practice of the present invention.

The present invention also provides a method wherein the genetic marker is a combination of the single nucleotide polymorphisms, or haplotypes, within the Jhdm2a gene, that is correlated with an aspect of obesity, diabetes (e.g., type II diabetes), metabolic syndrome, hyperinsulinemia, insulin resistance, glucose intolerance, hyperglycemia and/or hyperlipidemia.

The present invention is explained in greater detail in the following non-limiting Examples.

### Example 1

### Generation of Jhdm2a^{G/G} Mice

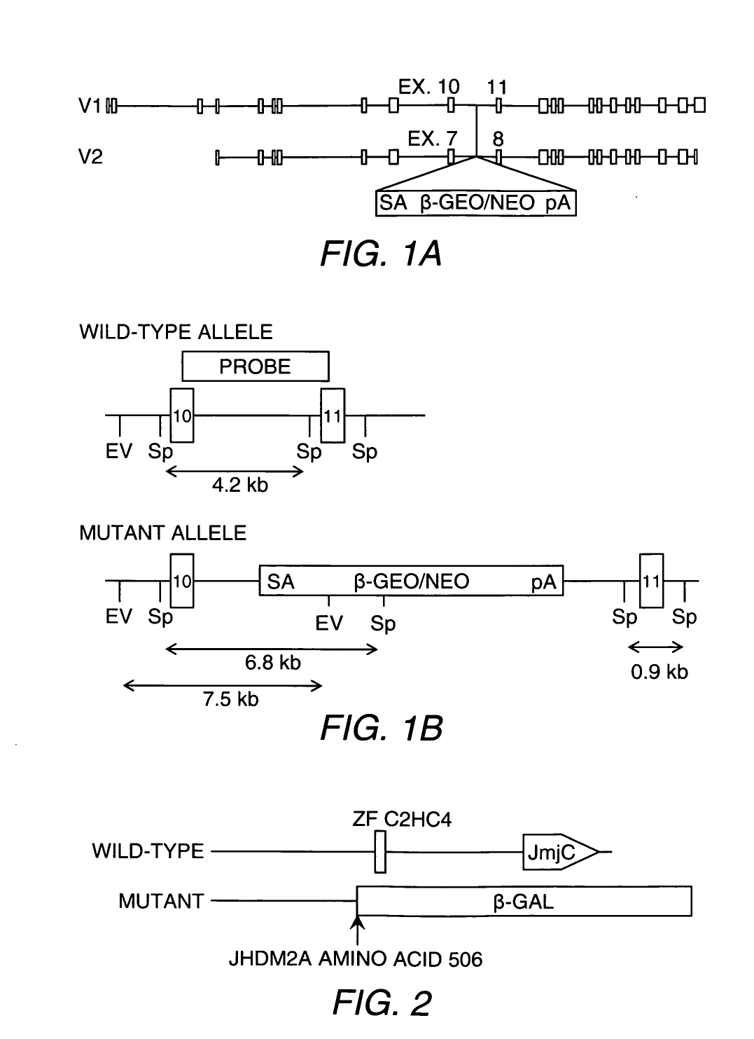

An embryonic stem (ES) cell clone with Jhdm2a gene trap allele (YHA186) was purchased from BayGenomics. Disruption of the Jhdm2a gene in ES cells was accomplished by the insertion of a β-Geo/Neo cassette in intron 10 (or intron 7 of splicing variant 2), see FIGS. 1A and 1B. The Jhdm2a mutant allele encodes the first 506 amino acids fused to β-GEO, and given the catalytic JmjC domain is deleted, the mutant protein would not be enzymatically active, see FIG. 2. The ES cells were then injected into C57BU6 blastocysts and chimeras and heterozygous (Jhdm2a^{+/G}) mice were obtained using standard procedures. After confirmation of germ line transmission, the progeny were backcrossed to C57BU6 once and homozygous mice that carry the β-Geo alleles (Jhdm2a^{G/G}) were obtained by crossing heterozygous (Jhdm2a^{+/G}) pairs.

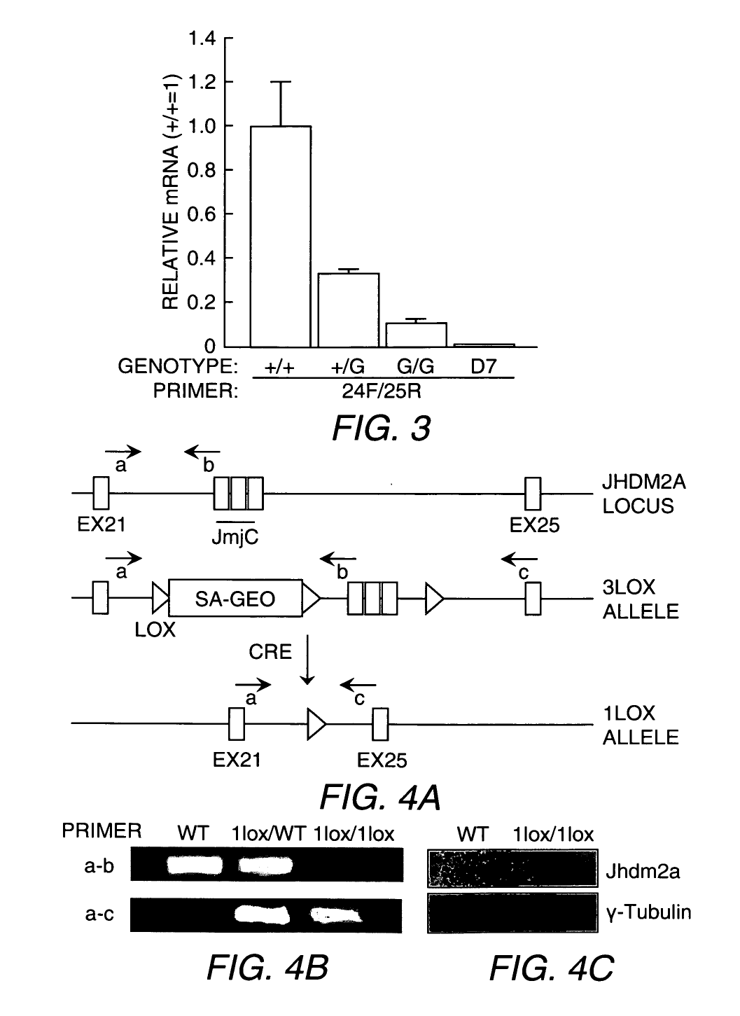

Homozygous mice, verified by Southern blot analysis, were viable and born at Mendelian ratios (data not shown). Expression of the Jhdm2a (exon1-10)-β-GEO fusion transcript in homozygous (Jhdm2a^{G/G}) and heterozygous (Jhdm2a^{+/G}) mice was confirmed by RT-PCR (data not shown). However, a low level (−10%) of wild-type mRNA was also detected in Jhdm2a^{G/G} mice, see FIG. 3. Whole mount β-Gal staining confirmed the expression of the β-Gal protein in testis in a dose-dependent manner (data not shown). Western blot analysis demonstrated that JHDM2A protein in testis was detected as a doublet, which corresponds to the full-length (V1, 152 KDa) and the splicing variant (V2, 139 KDa) (data not shown). Although the splicing variant was not detectable, lower levels of full-length JHDM2A protein still remained in the Jhdm2a^{G/G} mice (data not shown). Thus, the Jhdm2a^{G/G} mice were hypomorphic since they displayed some expression of the JHDM2A protein.

### Example 2

### Generation of Jhdm2a^{-/-} Mice

Using a bacterial artificial chromosome (BAC) clone, a targeting vector was constructed. In this vector, a loxP site and a β-Geo cassette flanked by two loxP sites were introduced into the mouse Jhdm2a locus, see FIG. 4A. E14 ES cells, derived from 129Sv strain that had undergone homologous recombination to have 3loxP sites, were isolated using standard procedures. Chimeric mice were mated with wild-type C57BU6 mice to generate F1 mice with a 3loxP allele. Heterozygous mice with 1lox allele lacking the JmjC domain were established by crossing the F1 mice with Ella-Cre mice in C57BL/6 background to obtain offspring with 1loxP allele (Jhdm2a^{+/-} mice). Jhdm2a^{-/-} (KO) mice in which exons 22-24, encompassing the catalytic JmjC domain, are floxed by two loxP sites, were obtained by mating heterozygous (Jhdm2a^{+/-}) mice. Correct genotypes were confirmed by PCR amplification of genomic DNA (FIG. 4B) and lack of expression of the JHDM2A protein in the KO mice was confirmed by western blot analysis (FIG. 4C).

### Example 3

### Animal Experiments

All results are presented as the mean±standard error (S.E.). Statistical comparisons were by Student's t tests. Statistical significance was set at p<0.05, where NS indicates not significant.

All animal experiments were performed according to procedures approved by the Institutional Animal Care and Use Committee (IACUC). Mice were maintained on a diet of standard rodent chow or a high-fat diet containing 60% fat-derived calories (58Y1, TestDiet, Richmond, Ind.) with 12 hour light and dark cycles. Body weight was measured weekly for a period of 8 months. For diet-induced obesity, 4 week-old mice were fed a high-fat diet for 2 months and body fat was measured weekly. The food intake of mice was measured using singly housed mice. Before measurement of food intake, the mice were acclimated to the housing environment for at least a week, and the intake data was collected during a 2-week period.

Primer sequences used for genotyping, qRT-PCR and ChIP analysis are listed in Table 1.

TABLE 1

| | | | SEQ |
|---|---|---|---|
| | | | ID |
| Gene | | Primer Sequence | NO: |
| Jhdm2a genotype a | | CCAAATGCCCAAGGCTTTAC | 1 |
| Jhdm2a genotype b | | CTGCACCGAATTCTCTTCCG | 2 |
| Jhdm2a genotype c | | CCACATACACAAACTTCTCTTAG | 3 |
| Ucp1 ChlPa | Forward | TCACCCTTGACCACACTGAA | 4 |
| | Reverse | GTGAGGCTGATATCCCCAGA | 5 |
| Ucp1 ChlPb | Forward | GCCAGGCCTGTAAACTCTGA | 6 |
| | Reverse | TGGGTTAAAAATGGCCTCTG | 7 |
| PPARα ChlPa | Forward | TTCCGAACCATTCTTTCCAG | 8 |
| | Reverse | GCTGCCTTCTTTTGCAGAGT | 9 |
| PPARα ChlPb | Forward | GAGACCCTGGAGATGGTTGA | 10 |
| | Reverse | GTGTGCTTGTGTGCATGTGA | 11 |
| Acox1 ChlP | Forward | CGTCAGTCAAGTCGGCAATA | 12 |
| | Reverse | CAAATTGGAGCGAAAGGGTA | 13 |
| Mcad ChlP | Forward | GAGAGCTGGACCCGAATCTA | 14 |
| | Reverse | CCGGAGCCATAAAGAATCTG | 15 |
| Scp2 ChlP | Forward | GTCAACTTGCCCCAAACCTA | 16 |
| | Reverse | GTGGAGTAGACCCTCCCACA | 17 |
| PGC-lα ChlPa | Forward | TCGGGATTGTCACGTAATGA | 18 |
| | Reverse | CTCCGGTACCGTCCAG | 19 |
| PGC-lα ChlPb | Forward | GTGGAAGCAGGGTCAAAATC | 20 |
| | Reverse | GCCATCAGTAAAGGGTGCAT | 21 |
| Jhdm2a | Forward | TGACCCTATCCACGATCAGA | 22 |
| | Reverse | TCCTGAGTAAGCCAGAAGCA | 23 |
| PPARα | Forward | TGCCAAGGAGTCGAGGATGT | 24 |
| | Reverse | TCGGCACCAGGAACCAA | 25 |
| PPARδ | Forward | CGCAAGCCCTTCAGTGACAT | 26 |
| | Reverse | CGCATTGAACTTGACAGCAAA | 27 |
| PGC-lα | Forward | TGCGGGATGATGGAGACA | 28 |
| | Reverse | GCGAAAGCGTCACAGGTGTA | 29 |
| PGC-1β | Forward | TCGAAATCTCTCCAGTGACATGA | 30 |
| | Reverse | CTACAATCTCACCGAACACCTCAA | 31 |
| UCP1 | Forward | CACCTTCCCGCTGGACACT | 32 |
| | Reverse | CCTGGCCTTCACCTTGGAT | 33 |
| UCP2 | Forward | GCCCCTTCACCTCTTTAGCA | 34 |
| | Reverse | CCAAGCACTGGGAAGGTCTAAC | 35 |
| MCAD | Forward | TGGATCTGTGCAGCGGATT | 36 |
| | Reverse | GGGTCACCATAGAGCTGAAGACA | 37 |
| LCAD | Forward | GGCAAAATACTGGGCATCTGA | 38 |
| | Reverse | CTCCGTGGAGTTGCACACAT | 39 |
| VLCAD | Forward | ACCTTGCCAGGGCCTGAT | 40 |
| | Reverse | TGGCCTGGTCACCGGTAA | 41 |
| AQP7 | Forward | GGTGATGGCGAAGAGACACA | 42 |
| | Reverse | GCTTCCTGGATGAGGCATTC | 43 |
| ACOX1 | Forward | CCACTCAAACAAGTTTCATACACATT | 44 |
| | Reverse | GGATGTGACCCTTGGCTCTGT | 45 |
| SCP2 | Forward | CGCTGCCGCGATTCTG | 46 |
| | Reverse | TTTGGACTGCAGGCCGTACT | 47 |
| RXRα | Forward | GGATGGTGATGCATCTTTTGG | 48 |
| | Reverse | ATGGCCCGTGTGGATCTTT | 49 |
| PPARγ | Forward | CCATGAGATCATCTACACGATGCT | 50 |
| | Reverse | CCCTCTGAGATGAGGACTCCAT | 51 |
| ATF2 | Forward | TGCTATGGTCTCAGTGTCATCTCTT | 52 |
| | Reverse | CTCTCAAGGGCATTCCATACG | 53 |
| CPT2 | Forward | CCCAGACATCTCGGTTCTCACT | 54 |
| | Reverse | GCCCTGTGCCCGAGTTT | 55 |
| p300 | Forward | TGGCACCGGGCACTTG | 56 |
| | Reverse | CAAAGGCTGCAAACGGAAAA | 57 |
| SRC1 | Forward | GGTTCCACAGACAAAGTGGTGAT | 58 |
| | Reverse | CTCTGACTGAGCGGCATTAAAATT | 59 |
| HPRT | Forward | TGAAAGACTTGCTCGAGATGTCA | 60 |
| | Reverse | CACACAGAGGGCCACAATGT | 61 |
| 36B4 | Forward | GCCAGCTCAGAACACTGGTCTA | 62 |
| | Reverse | ATGCCCAAAGCCTGGAAGA | 63 |
| ERRα | Forward | GGAGTACGTCCTGCTGAAAGCT | 64 |
| | Reverse | CAGCATCTTCAATGTGCACAGA | 65 |
| ATP5J | Forward | TCGGGACTCAGTGCAAGTACAG | 66 |
| | Reverse | ACAGAGGAGAGCCTGAAGATCCT | 67 |
| COX7A | Forward | GGCTCTGGTCCGGTCTTTTAG | 68 |
| | Reverse | TTCTCTGCCACACGGTTTTCT | 69 |
| NDUFB5 | Forward | TGTGGGCCTTTCAGCTTCAT | 70 |
| | Reverse | CCCAGACCTCCAGGTTTCATT | 71 |
| NRF1 | Forward | GCCGTCGGAGCACTTACTG | 72 |
| | Reverse | GGTACATGCTCACAGGGATCTG | 73 |
| NRF2 | Forward | GATCAGGCGACATGTTTAACGTT | 74 |
| | Reverse | AGAGCCCAGTCAAACCCTTTC | 75 |

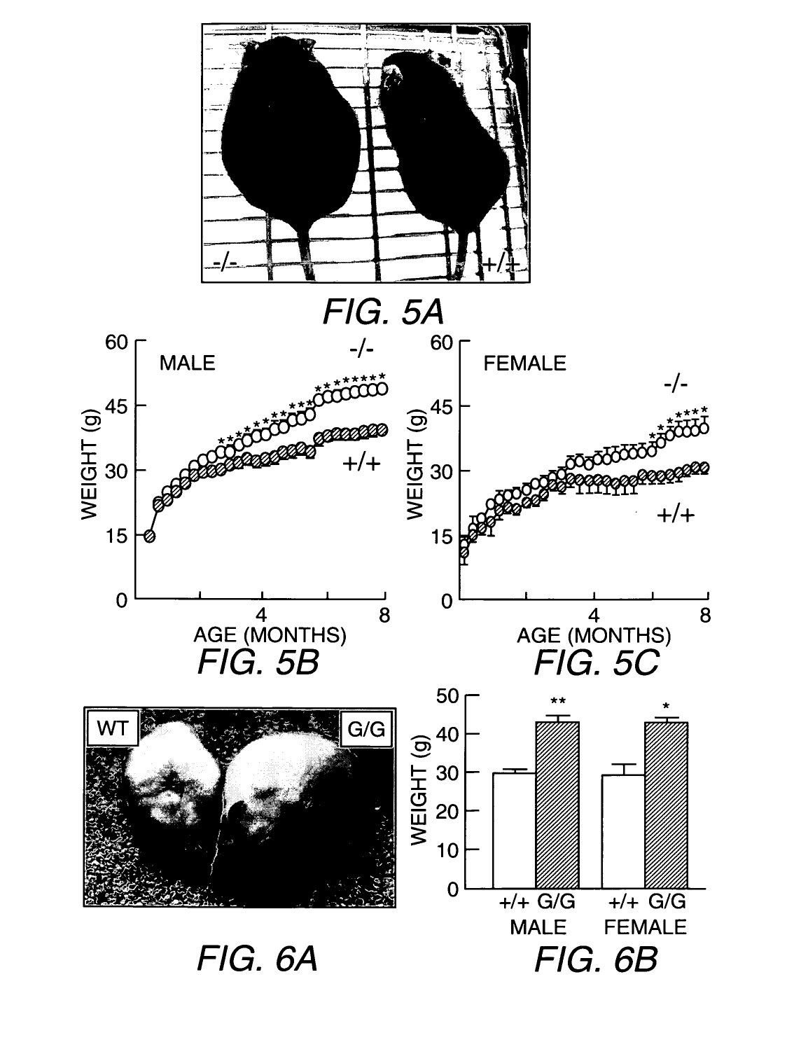

The results demonstrated that both male and female Jmdm1a^{-/-} mice become obese in adulthood when compared with their wild-type littermates, see FIGS. 5A-5C. The obese phenotype was also observed in Jhdm2a knockout mice backcrossed onto a C57BL/6 background (data not shown), as well as in the hypomorphic Jhdm2a mice obtained as described above in Example 1, see FIG. 6, indicating that Jhdm2a deficiency leads to obesity irrespective of the genetic background. Moreover, similar to the hypomorphic Jhdm2a mice, the complete knockout mice exhibited spermatogenesis defects (data not shown).

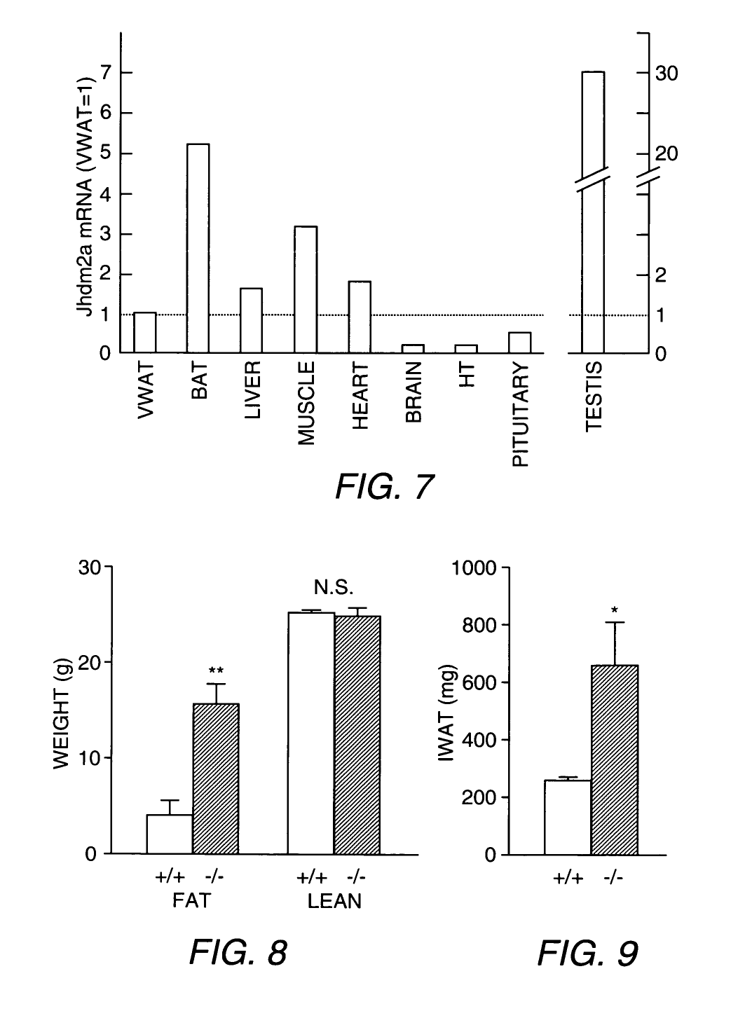

Jhdm2a 1lox/1lox (knockout) mice were confirmed by PCR (FIG. 4B) and western blot analysis (FIG. 4C). Jhdm2a mRNA levels in different mouse organs were determined using quantitative RT-PCR (qRT-PCR). Primers specific for HPRT mRNA were used for normalization. Consistent with a role for Jhdm2a in energy homeostasis, Jhdm2a was expressed at a relatively higher level in mitochondria-rich organs, such as brown adipose tissues, skeletal muscle, and heart, as compared with the white adipose tissue, see FIG. 7. In contrast, Jhdm2a expression in the brain was almost undetectable. The highest expression was detected in testis.

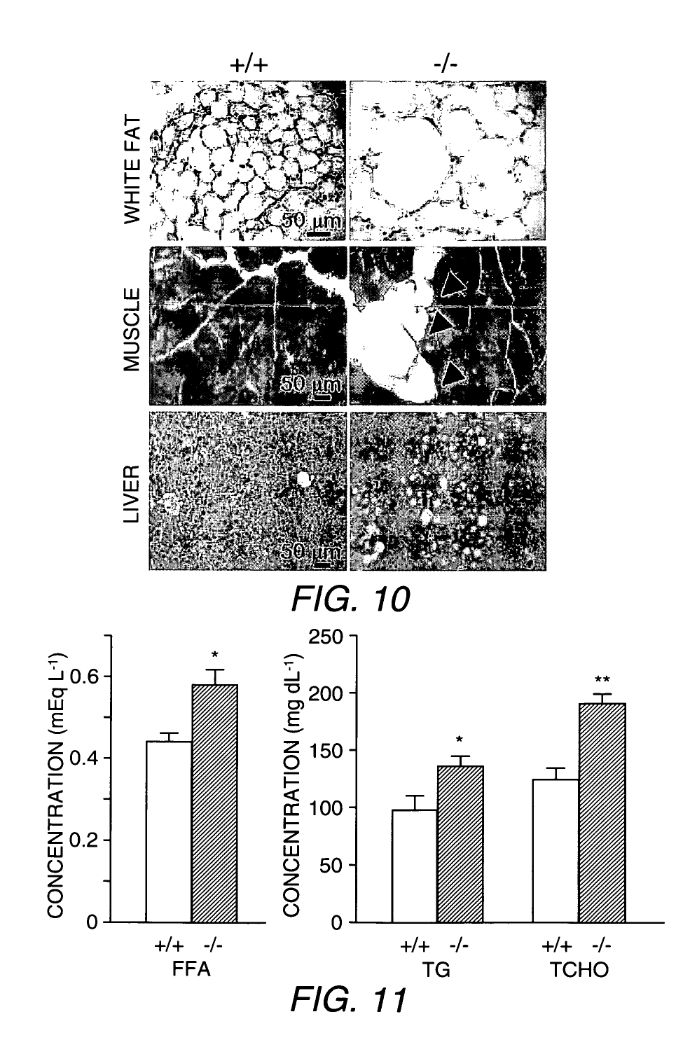

Body fat deposition in 4-month-old KO mice was determined using Magnetic Resonance Imaging. The analysis revealed marked body fat deposition in KO mice. However, non-adipose tissues (lean) were comparable between wild-type and Jhdm2a knockout mice, see FIG. 8. Additionally, the weight of visceral fat tissues such as inguinal white adipose tissue (IWAT) was also significantly increased in the KO mice, see FIG. 9. Similar to that observed in human obesity, large fat droplets were observed in white adipose tissue (WAT), as well as in muscle and liver. see FIG. 10. Fat deposition surrounding the central vein in liver, a typical feature of human fatty liver, as well as intra-muscle fat deposition, was also evident in the KO mice (data not shown). Furthermore, serum lipid content, including free fatty acid (FFA), triglyceride (TG), and total cholesterol (TCHO), was found to be significantly higher in KO mice, see FIG. 11. The blood level of triglyceride and total cholesterol were analyzed in the Animal clinical chemistry and gene expression core facility at the University of North Carolina. Serum-free fatty acid was measured using the HR series NEFA-HR kit (Wako Diagnostics).

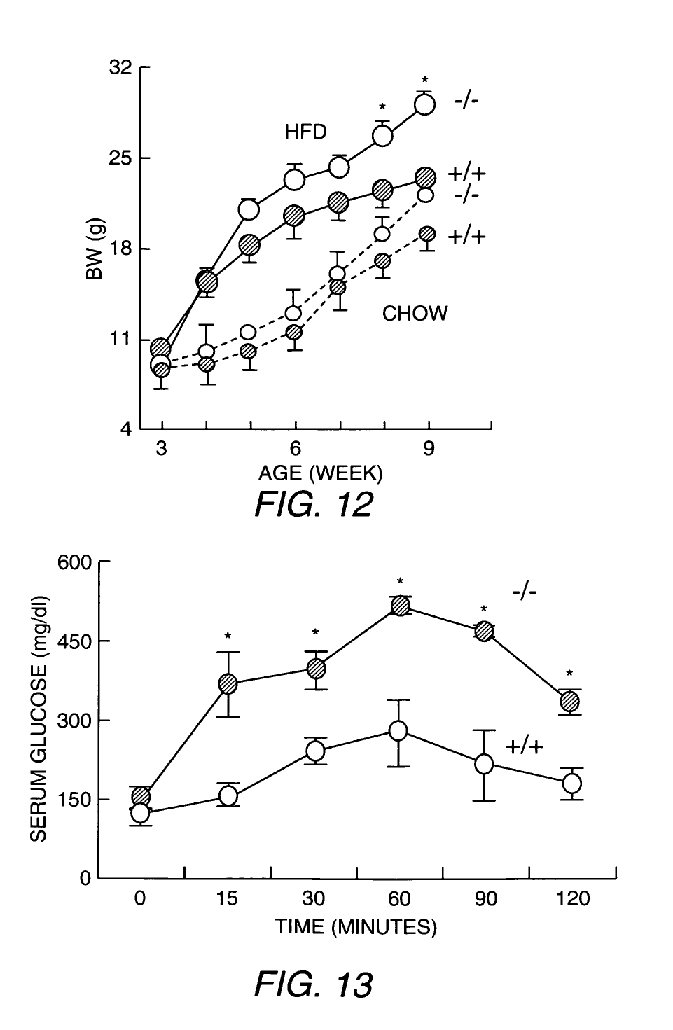

To determine whether Jhdm2a KO mice were prone to diet-induced obesity, 4-week-old mice were fed a high-fat diet (HFD) for 2 months after weaning and their body weight was measured weekly. The results shown in FIG. 12 indicate HFD accelerated the onset of obesity in KO mice, when compared with normal chow. Collectively, these findings suggest that loss of JHDM2A function results in abnormal fat metabolism and obesity.

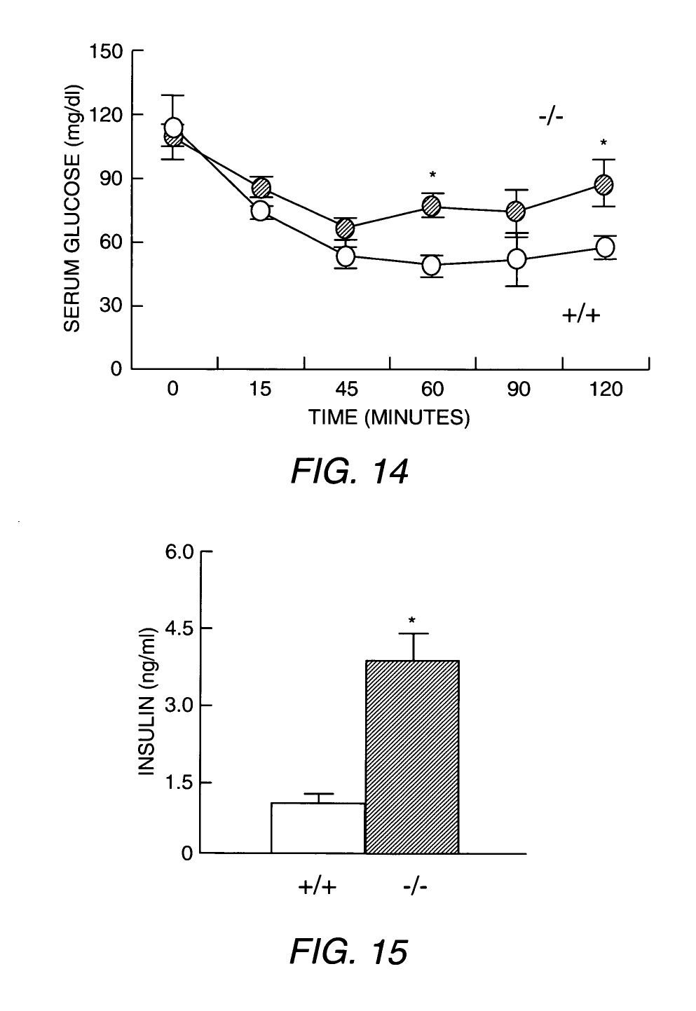

In humans, excessive body fat deposition is closely linked to systemic insulin resistance, and diet-induced obesity is a primary risk factor for Type 2 diabetes (Qatanani, Genes Dev. 21, 1443 (2007); Kahn, J. Clin. Invest. 106, 473 (2000)). To determine whether Jhdm2a-deficiency also causes systemic insulin resistance, glucose and insulin tolerance tests (GTT and ITT, respectively) were performed. Glucose (1.5 mg/g) was intra-peritoneously infused for GTT. To measure glucose levels, blood was collected from the tail before and 15, 30, 60, 90, and 120 minutes after infusion. Glucose levels were determined using an Assure3 glucose meter (HYPOGUARD). For ITT, mice were fasted for 4 hours prior to intra-peritoneal injection of insulin (1 IU/kg). Body composition was evaluated by EchoMRI-100 (Echo Medical Systems). GTT showed that Jhdm2a KO mice were glucose-intolerant, see FIG. 13, and ITT indicated Jhdm2a-deficiency results in reduced insulin sensitivity, see FIG. 14. The significantly higher plasma insulin level, as shown in FIG. 15, and the existence of islet hyperplasia in the pancreas of KO mice (data not shown) are consistent with hyperinsulinemia resulting from insulin resistance. These data indicate that loss of JHDM2A function impairs glucose metabolism. In summary, the obesity phenotypes observed in Jhdm2a KO mice, including increased visceral fat deposition, hyperlipidemia and systemic insulin resistance, closely mimic human metabolic syndrome.

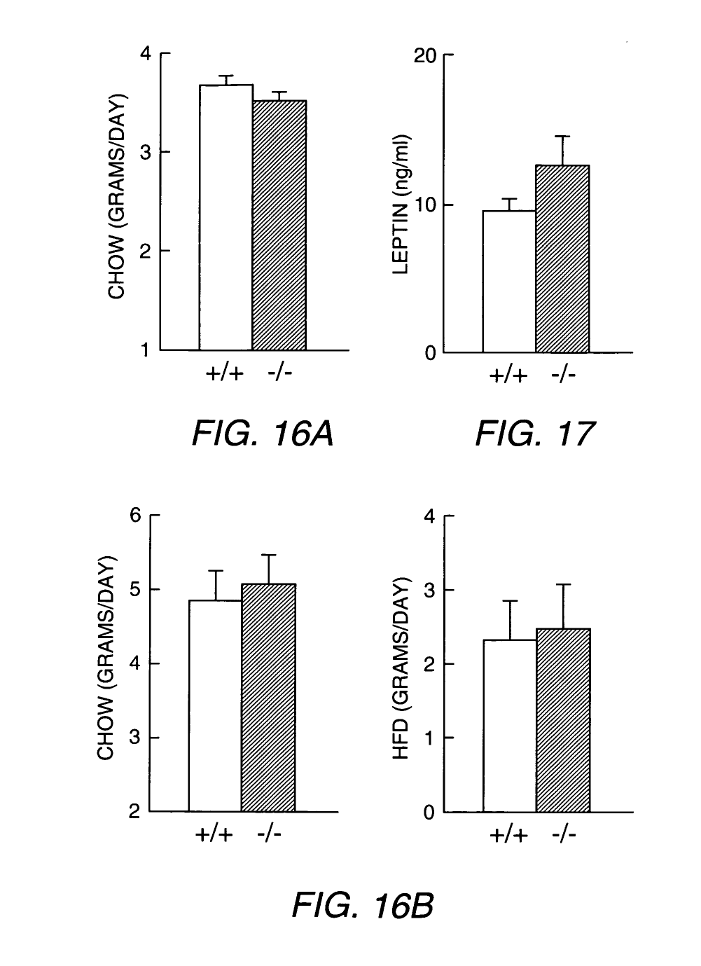

The body weight of an animal is maintained through a balance between food intake and energy expenditure (Spiegelman, Cell 104, 531 (2001); Evans, Nat Med 10, 355 (2004)). To understand how Jhdm2a-deficiency results in obesity, food intake was analyzed before the onset of obesity using 6-week-old mice. It was found that caloric intake was not elevated in Jhdm2a KO mice, see FIG. 16A. Similarly, the 16-week-old Jhdm2a knockout mice did not show increased food intake, regardless of the fat content present in the diet, see FIG. 16B. In addition, the serum leptin level was not significantly altered in 6-week-old Jhdm2a knockout mice before they became obese, see FIG. 17. Leptin in serum was assayed using the mouse leptin ELISA kit (CRYSTAL CHEM) according to the manufacture's instructions.

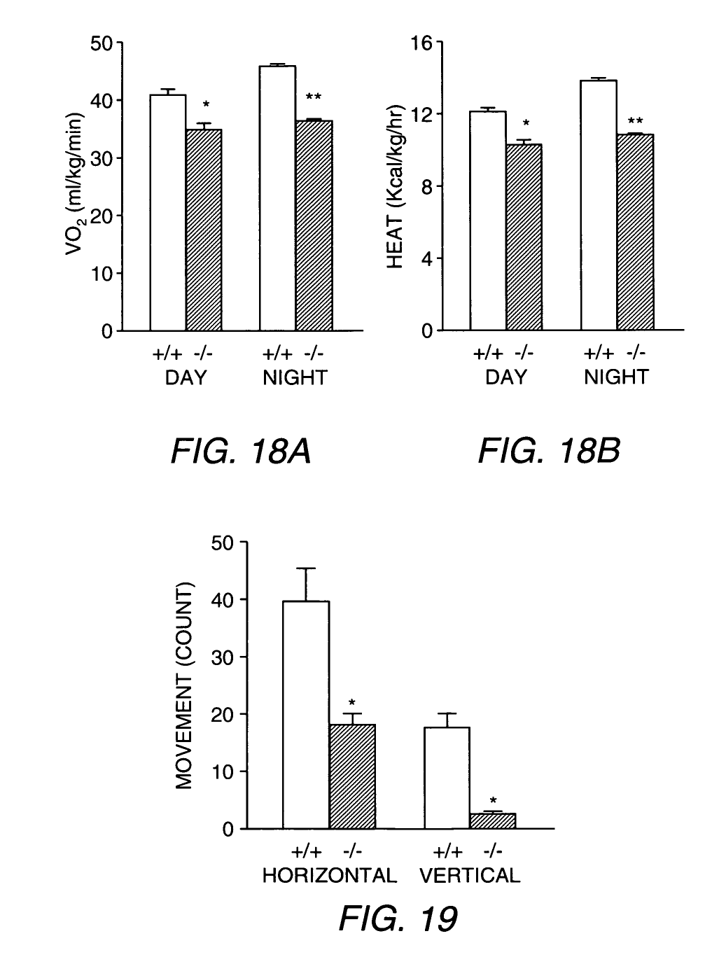

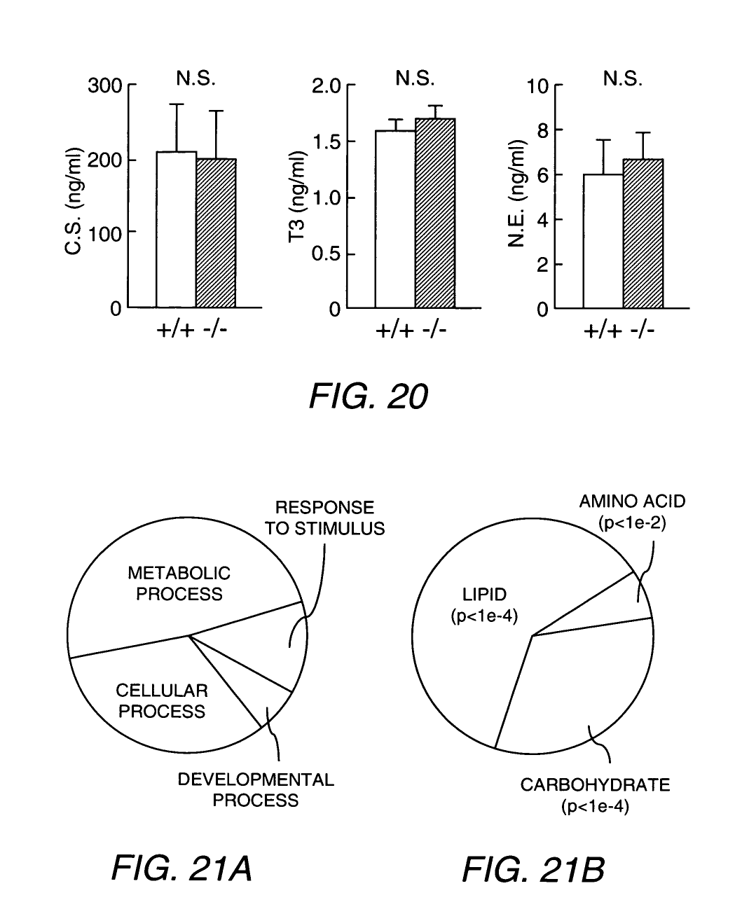

The effect of Jhdm2a deficiency on energy expenditure was analyzed. Systemic energy expenditure includes basal metabolic rate, physical activity, and adaptive thermogenesis (Spiegelman, Cell 104, 531 (2001)). Metabolic studies were performed using TSE LabMaster calorimetry Module (TSE-System) in the Clinical Nutrition Research Unit at the University of North Carolina. For respiration measurements, cultured cells were resuspended in PBS. Oxygen consumption was measured using a Clarke-type electrode (Diamond General) at baseline, and after treatment with 10 μM isoproterenol (ISO) (Wu, Cell 98, 115 (1999)). For normalization of the respiration rates, an aliquot of the cells was lysed and protein concentration was measured. It was found that loss of JHDM2A function results in decreased metabolic rate as evidenced by decreased oxygen (O₂) consumption and heat generation, see FIGS. 18A and 18B. Loss of JHDM2A function also resulted in decreased physical activity as evidenced by reduced horizontal and vertical movement, see FIG. 19. The decreased energy expenditure of the Jhdm2a KO mice was not a result of abnormal metabolic hormone secretion. Plasma hormone levels such as corticosterone (C.S.), triiodothyronine (T3), and norepinephrine (N.E.), which influence physical activity or metabolic rate (Evans, Nat. Med. 10, 355 (2004)), were measured at Vanderbilt Diabetes Research and Training Center and were not significantly altered in the Jhdm2a KO mice, see FIG. 20 and Table 2. Adiponectin in serum was assayed using the Circulex mouse adiponectin ELISA kit (MBL) according to the manufacture's instructions.

TABLE 2

| Plasma Hormone Level | | | |
|---|---|---|---|
| (ng/ml) | WT | KO | p |
| Norepinephrine | 6.0 ± 1.5 | 6.6 ± 1.2 | 0.778 |
| Epinephrine | 11.7 ± 2.8 | 7.9 ± 1.7 | 0.312 |
| Testosterone | 0.4 ± 0.08 | 0.4 ± 0.07 | 0.937 |
| Insulin | 1.0 ± 0.2 | 3.9 ± 1.2 | 0.04 |
| T3 | 1.6 ± 0.1 | 1.7 ± 0.1 | 0.3 |
| Corticosterone | 209.7 ± 63.7 | 200.6 ± 59.5 | 0.921 |
| Leptin (feeding) | 10.5 ± 4.3 | 27.9 ± 17.2 | 0.005 |
| Leptin (fasting) | 3.9 ± 1.4 | 19.4 ± 6.6 | 0.007 |
| Adiponectin | 27.4 ± 3.1 | 28.3 ± 5.0 | 0.885 |

Given that the human counterpart of mouse Jhdm2a has been previously demonstrated to function as a transcriptional co-activator (Yamane, Cell 125, 483 (2006)), gene expression profiles of wild-type and Jhdm2a knockout skeletal muscles were compared using AFFYMETRIX microarray technology. The analysis was performed for skeletal muscle because of the high expression of Jhdm2a and also its role in energy expenditure.

Seven μg of total RNA was used to synthesize cDNA. A custom cDNA kit from Life Technologies was used with a T7-(dT)24 primer for this reaction. Biotinylated cRNA was then generated from the cDNA reaction using the BIOARRAY High Yield RNA Transcript Kit. The cDNA was then fragmented in fragmentation buffer (5× fragmentation buffer: 200 mM Tris-acetate, pH 8.1, 500 mM KOAc, 150 mM MgOAc) at 94° C. for 35 minutes before the chip hybridization. Fifteen μg of fragmented cDNA was then added to a hybridization cocktail (0.05 μg/μl fragmented cDNA, 50 pM control oligonucleotide B2, BioB, BioC, BioD, and cre hybridization controls, 0.1 mg/ml herring sperm DNA, 0.5 mg/ml acetylated BSA, 100 mM MES, 1 M [Na+], 20 mM EDTA, 0.01% TWEEN 20). Ten μg of cDNA was used for hybridization. Arrays were hybridized for 16 hours at 45° C. in the GENECHIP Hybridization Oven 640. The arrays were washed and stained with R-phycoerythrin streptavidin in the GENECHIP Fluidics Station 400. After this, the arrays were scanned with the HEWLETT PACKARD GENEARRAY Scanner. AFFYMETRIX GENECHIP Microarray Suite 5.0 software was used for washing, scanning, and basic analysis. Sample quality was assessed by examination of 3′ to 5′ intensity ratios of certain genes. Data analysis was carried out in GENESPRING GX 10.0 software (Agilent Technologies) using the RMA algorithm. Probes exhibiting at least a 2-fold reduction in mRNA level in Jhdm2a KO samples versus WT samples were subjected to pathway analysis and gene ontology enrichment analysis with significance threshold set to p<0.05. Data mapping onto the PPAR pathway was carried out using GENMAPP 2.0 (Gladstone Institutes).

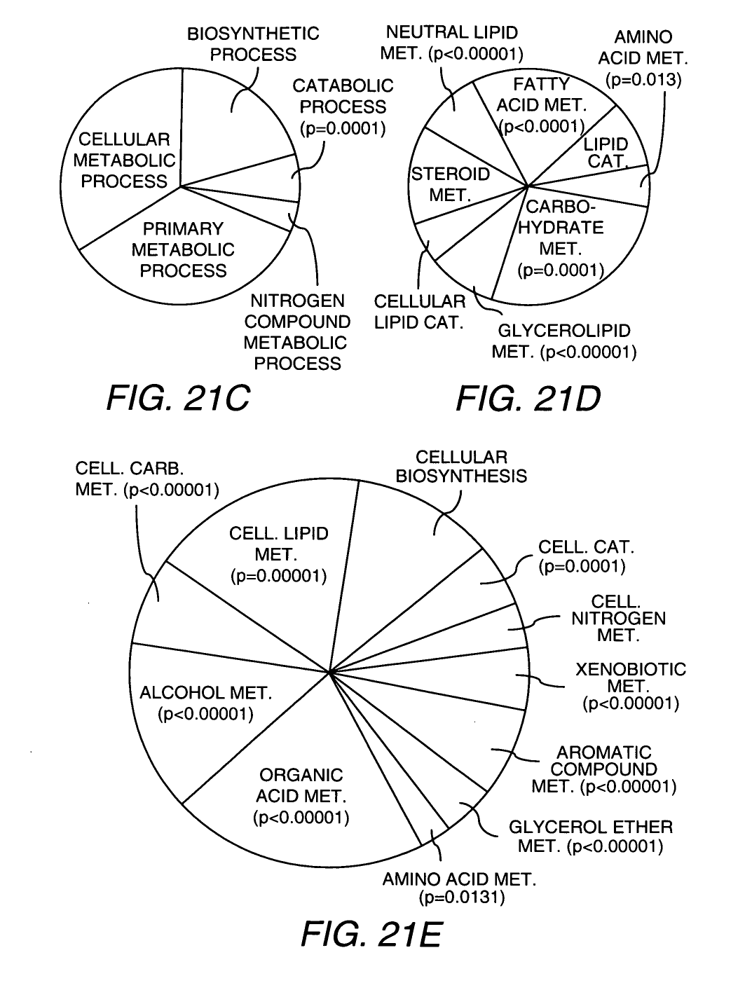

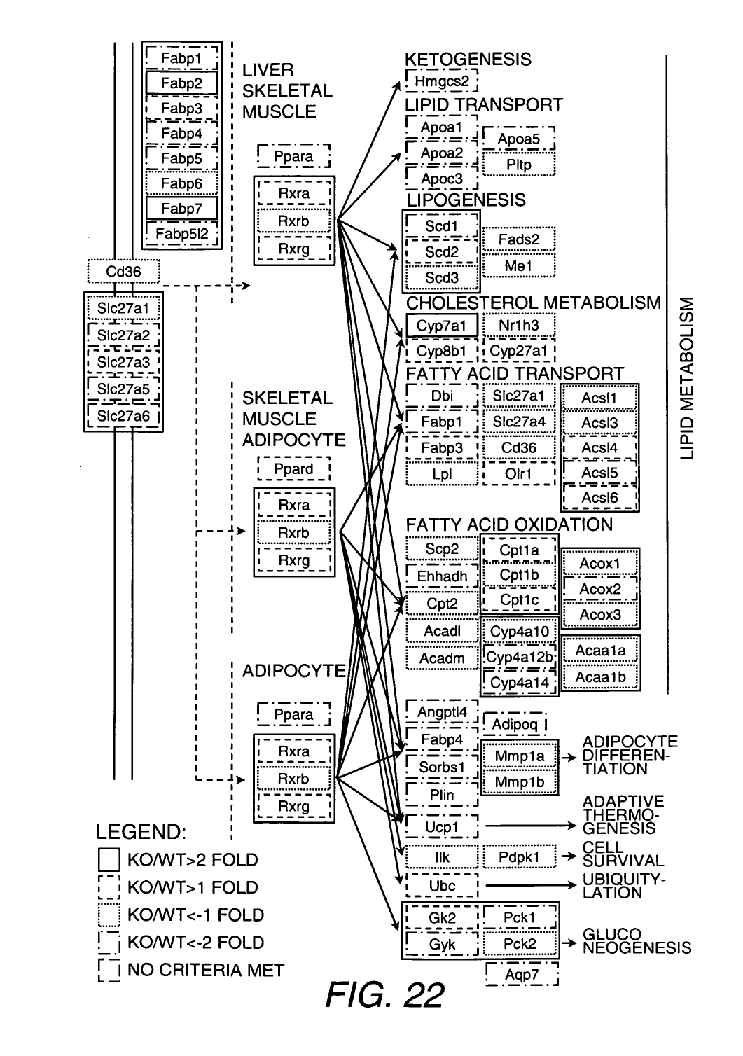

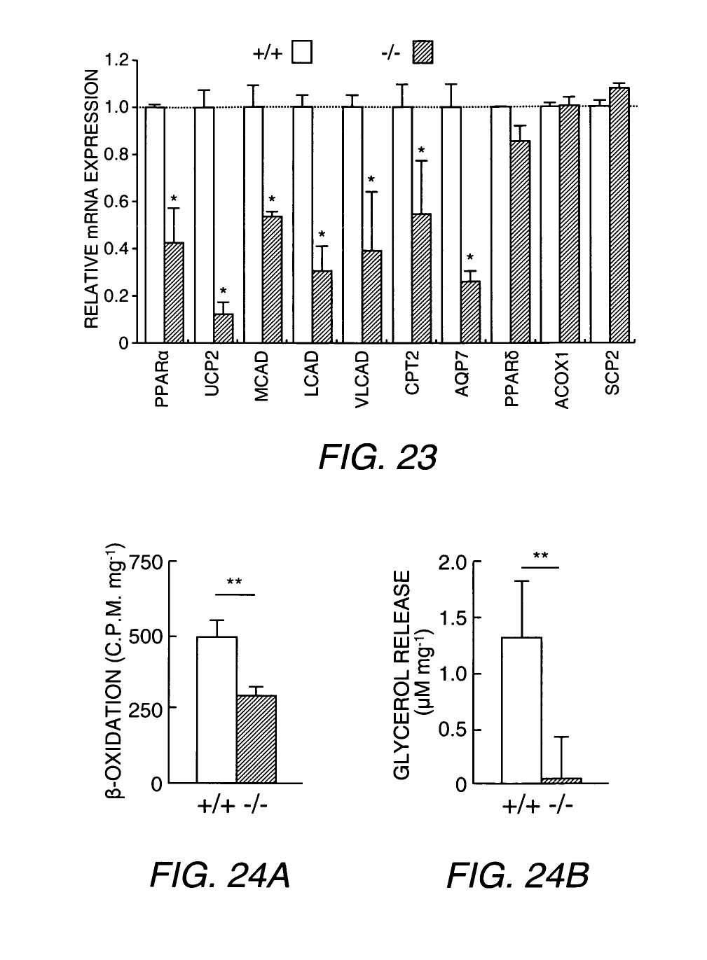

Of the 43,000 probes analyzed, 602 probes (1.4%) were down-regulated at least two-fold and 220 probes (0.05%) were up-regulated at least two-fold in jhdm2a knockout soleus muscle. Gene ontology enrichment analysis indicated that the largest proportion of down-regulated genes were involved in metabolic processes (P=0.03), see FIG. 21A, whereas genes involved in lipid metabolism were the most enriched (P<1×10⁻⁴), see FIGS. 21B-21E. Interestingly, pathway analysis showed that a significant proportion of the genes involved in PPAR signaling were down-regulated in response to the Jhdm2a knockout (P<1×10⁻¹¹), see FIG. 22. Quantitative PCR with reverse transcription (qRT-PCR) confirmed the down-regulation of many genes involved in the PPAR pathway, including PPARα, Ucp2, MCAD (also known as Acadm), LCAD (Acadl), VLCAD (Acadvl) and Aqp7 but not PPARδ, see FIG. 23. Consistent with the fact that multiple rate-limiting enzymes for fatty acid oxidation, such as MCAD, LCAD and VLCAD, were down-regulated in the Jhdm2a knock-out skeletal muscle, β-oxidation of palmitic acid was significantly decreased in primary myocytes derived from the Jhdm2a knock-out newborn (FIG. 24A). Measurement of in vitro β-oxidation of [1-¹⁴C] palmitic acid was performed using primary muscle cells or C2C12 myotubes. Cells were exposed to [1-¹⁴C] palmitic acid (0.2 μCi/ml) for 60 minutes at 30° C. and acidified with 1N HCl for an additional 30 minutes at 30° C. The produced ¹⁴CO₂ was trapped with a paper filter pre-soaked with NaOH. The filter was then transferred to a scintillation vial, and the total level of released radiolabeled CO₂ was measured by scintillation counting.

Consistent with down-regulation of Aqp7 (FIG. 23), glycerol release in response to a β-adrenergic agonist was impaired in the Jhdm2a knock-out soleus muscle, see FIG. 24B. In vitro glycerol release was measured by surgically isolating soleus muscle and intrascapular brown fat, incubating the tissue pieces in DMEM containing 2% fatty acid-free bovine serum albumin with or without 10 μM isoproterenol (Sigma-Aldrich) at 37° C. for 2 hours, and measuring glycerol content using a commercial kit (Zenbio). These results are consistent with previous demonstrations that PPARα has an essential involvement in fatty acid metabolism (Reddy, et al., Annu. Reverse Nutr. 21, 193-230 (2001); Bedu, et al., Biochem. Biophys. Res. Commun. 357, 877-881 (2007); Finck, et al., Cell Metab. 1, 133-144 (2005)). Notably, the results also indicate that the impaired expression of PPARα and its downstream target genes might be one of the causes of abnormal fat accumulation in the Jhdm2a knock-out mice, because previous studies have shown that PPARα-deficient mice are prone to diet-induced obesity (Finck, et al., Cell Metab. 1, 133-144 (2005)) and that Aqp7 deficiency causes obesity in adult mice (Hibuse, et al., Proc. Natl. Acad. Sci. USA 102, 10993 (2005)).

In skeletal muscles, leptin promotes energy expenditure such as fat oxidation through activation of AMPK signaling, a major energy sensor at the cellular level (Minokoshi, Nature 415, 339 (2002); Kahn, Cell Metab. 1, 15 (2005); Hardie, Bioessays 23, 1112 (2001)). Activation of AMPK signaling was determined by treating C2C12 myotubes with 800 μM AICAR (Sigma Aldrich) for 16 hours. To prepare the C2C12 myotubes, primary myoblasts were isolated and established from neonatal mice as reported (Rando, J Cell Biol 125, 1275 (1994)). The primary myoblasts were then cultured in DMEM with 2% horse serum for the differentiation to primary myotubes. C2C12 cells were cultured in DMEM containing 10% FCS. Consistent with increased serum leptin levels in the Jhdm2a-deficient mice, see FIG. 17, increased AMPK phosphorylation was observed in the Jhdm2a-deficient skeletal muscle and purified myotubes (data not shown). Given that AMPK-mediated PGC-1α induction in muscle plays an important role in transcription of genes involved in mitochondrial metabolic processes such as fat oxidation (Zong, Proc. Natl. Acad. Sci. USA 99, 15983 (2002); Lee, Biochem. Biophys. Res. Commun. 340, 291 (2006); Jager, Proc. Natl. Acad. Sci. USA 104, 12017 (2007)), these findings suggest that the positive effect on energy expenditure by leptin-induced AMPK activation, and subsequent activation of PGC-1α and its target genes, is antagonized by Jhdm2a-deficiency. The most reasonable explanation of the above results is that Jhdm2a-deficiency directly impairs PGC-1α expression and mitochondrial metabolism.

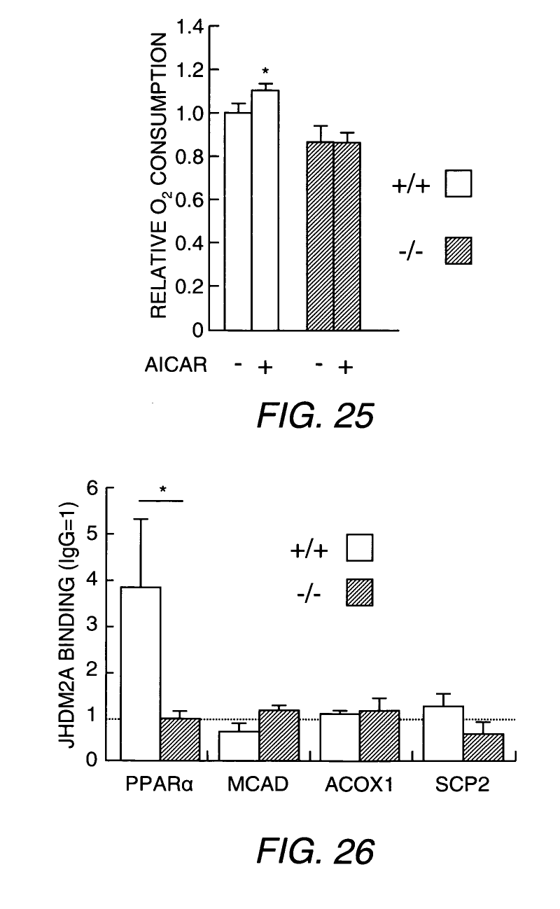

To gain further support for the involvement of JHDM2A in AMPK-mediated mitochondrial function, the effect of Jhdm2a-deficiency on AMPK activation-mediated respiration in primary myotubes was measured. The results shown in FIG. 25 indicate AICAR-induced AMPK activation results in increased respiration in wild-type cells, but not in Jhdm2a KO cells. Given that PGC-1α is required for the positive effects of AMPK on mitochondrial function in muscle cells (Jager, Proc. Natl. Acad. Sci. USA 104, 12017 (2007)), the above results indicate that JHDM2A plays a significant role in mitochondrial metabolism through AMPK-PGC-1α axis in skeletal muscles.

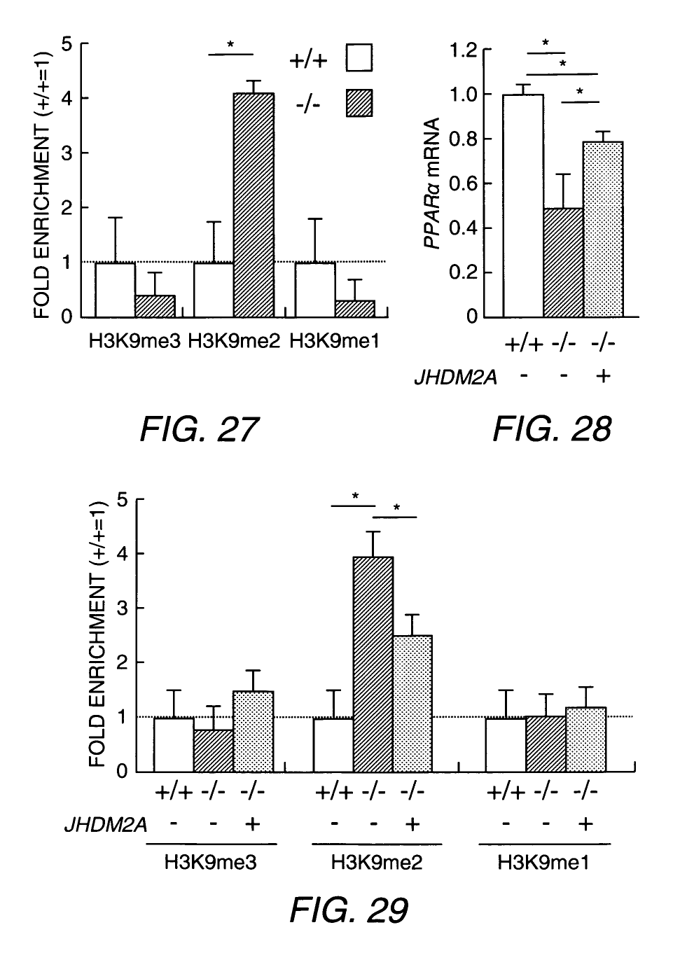

To understand how the loss of Jhdm2a function results in the down-regulation of PPARα and its target genes, the presence of Jhdm2a around defined PPRE sequences was analyzed using chromatin immunoprecipitation (ChIP) (Gulick, et al., Proc. Natl. Acad. Sci. USA 91:11012 (1994); Lopez, et al., Mol. Cell. Endocrinol. 205:169 (2003); Pineda Torra, et al., Mol. Endocrinol. 16:1013 (2002); Tugwood, et al., EMBO J. 11:433 (1992)). Results indicate that Jhdm2a binds to the PPRE of PPARα, but not to the PPREs of MCAD, Acox1 or Scp2, see FIG. 26. Consistent with the function of Jhdm2a as an H3K9me2 demethylase, the level of H3K9me2 at the region encompassing the PPRE of the PPARα gene was significantly increased in the Jhdm2a knock-out muscle cells, see FIG. 27.

To demonstrate that the reduced PPARα expression in Jhdm2a knock-out muscle cells was a cell-intrinsic effect, qRT-PCR and ChIP analysis were performed using in vitro-cultured primary myocytes derived from newborn wild-type and knock-out mice. Results shown in FIG. 28 demonstrate that PPARα is decreased by 50% in Jhdm2a knock-out myocytes. ChIP analysis showed increased levels of H3K9me2 at the PPARα PPRE of Jhdm2a knock-out myocytes, see FIG. 29. Notably, both PPARα expression and promoter H3K9me2 levels at PPRE could be partially rescued by overexpression of human JHDM2A (FIGS. 28 and 29). These data collectively indicate that PPARα is a direct target of Jhdm2a and that Jhdm2a plays an important part in lipid metabolism in skeletal muscle cells.

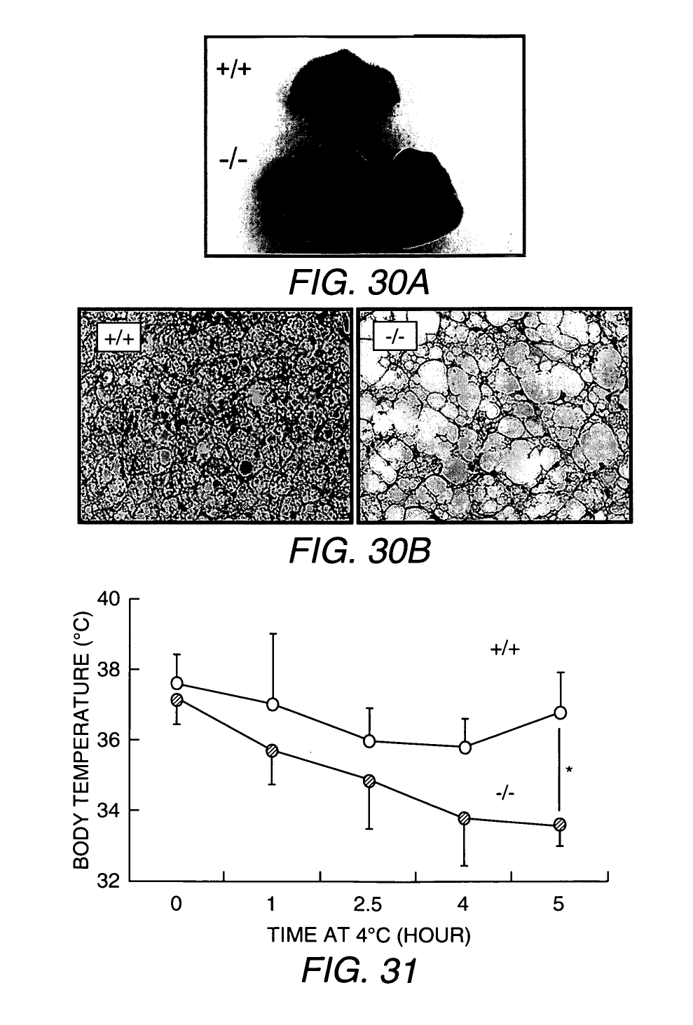

Because the brown adipose tissue (BAT) is the primary cellular component responsible for adaptive thermogenesis, the effect of Jhdm2a KO on BAT was analyzed. Brown adipose tissue was dissected 5 hours after cold exposure and subjected to quantitative RT-PCR. Total RNA was purified using an RNEASY or RNEASY lipid tissue kit (Qiagen). After DNase treatment, first-strand cDNA was synthesized using the IMPROM II Reverse Transcription System (Promega). Quantitative RT-PCR was performed using SYBR GREENER (Invitrogen). In addition to skeletal muscle, Jhdm2a is also highly expressed in BAT (FIG. 7). Jhdm2a deficiency resulted in the enlargement and accumulation of lipid droplets in the BAT, see FIG. 30. Consistent with the BAT abnormities, Jhdm2a knock-out mice showed defective adaptive thermogenesis, see FIG. 31. In contrast, Jhdm2a KO littermates could not maintain their body temperature when subjected to the same cold stress, see FIG. 31. For cold exposure, 12-week-old mice were individually housed in plastic cages at 4° C. for 5 hours. Core body temperature was intra-rectally monitored.

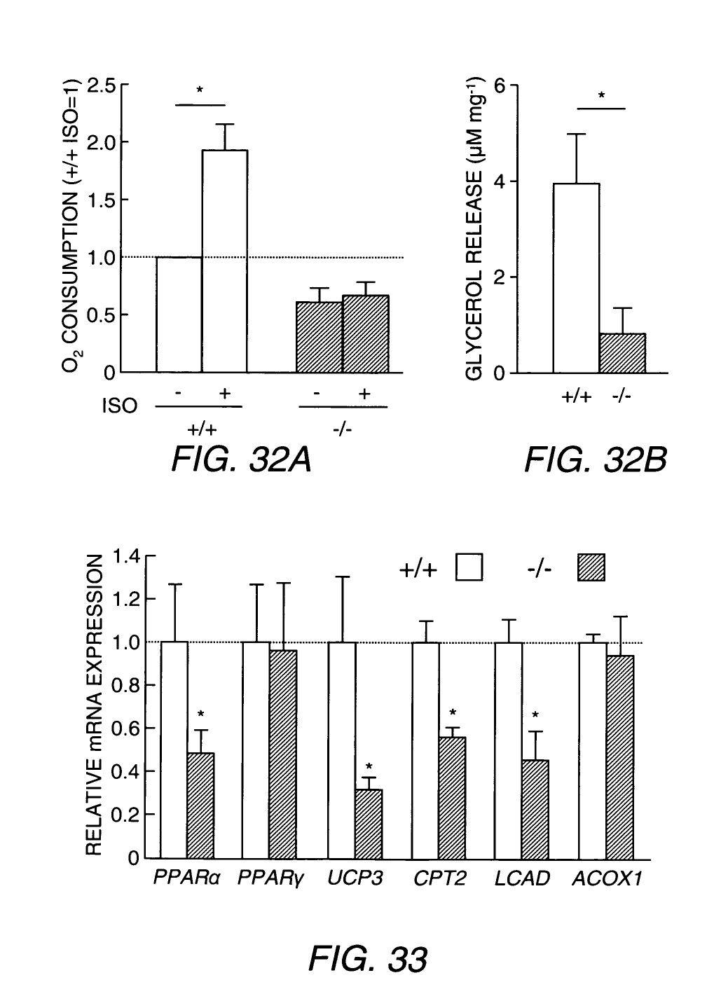

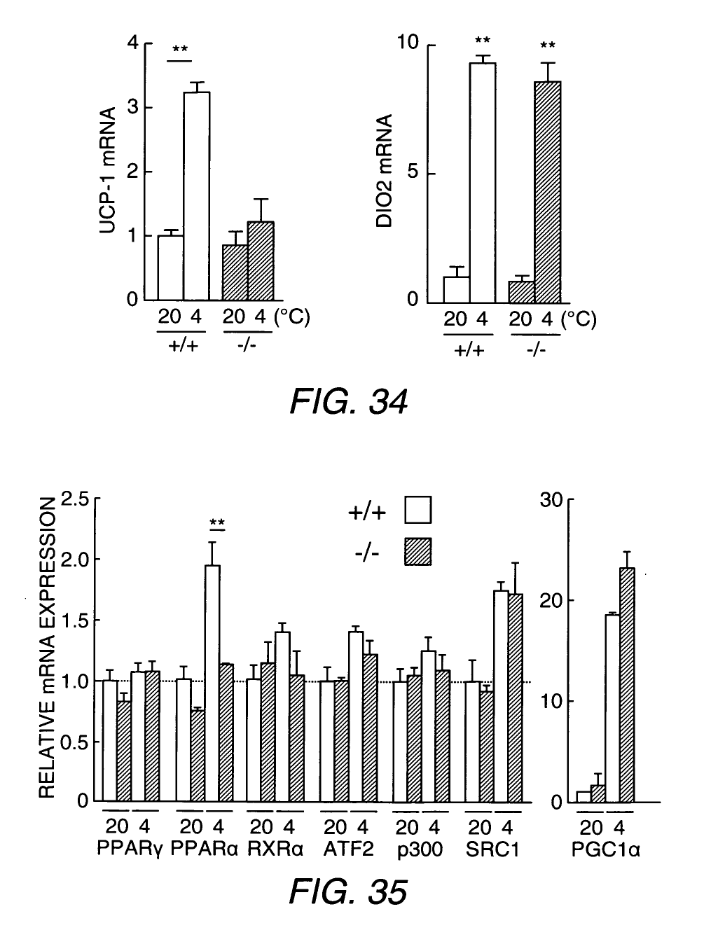

The phenotypic similarity between Jhdm2a knock-out mice and mice lacking β-adrenergic receptors (Bachman, et al., Science 297, 843 (2002)) raised the possibility that Jhdm2a might be a critical factor in β-adrenergic signaling. Consistent with this, β-adrenergic-stimulated oxygen consumption and glycerol release were greatly reduced in the Jhdm2a knock-out BAT, see FIGS. 32A and 32B. In addition, genes involved in mitochondrial functions including PPARα, Ucp3, Cpt2 and LCAD were also decreased in the Jhdm2a knock-out BAT, see FIG. 33 Furthermore, analysis of the expression of Ucp1 and Dio2, two key genes involved in thermogenesis in BAT (Lowell, Nature 404, 652 (2000)), demonstrated that cold-induced Ucp1 upregulation was almost completely blocked although Dio2 induction was not affected, see FIG. 34. Given the critical function of Ucp1 in cold sensitivity (Enerback, et al., Nature 387:90 (1997)), defective activation of Ucp1 by β-adrenergic signaling is probably one contributing factor of defective thermogenesis in the Jhdm2a knock-out BAT.

As one of the most important molecules involved in cold-induced thermogenesis in brown fat, the transcriptional regulation of Ucp1 has been extensively characterized (Lowell, et al. Nature 404, 652 (2000)). In addition to PPARα and PPARγ, other transcription factors and co-activators known to be involved in Ucp1 activation include Rxrα, Atf2, p300, Src1 and Pgc1α. qRT-PCR demonstrated that the expression of these genes was not significantly altered in the Jhdm2a knock-out BAT; however cold-induced upregulation of PPARα was defective in knock-out BAT, see FIG. 35. This indicates that the defect in cold-induced Ucp1 induction in Jhdm2a knock-out BAT may be mediated through PPARα, which is a direct target of Jhdm2a in muscle cells (FIG. 26).

### Example 4

### Jhdm2a Knockdown in Brown Adipose Cells

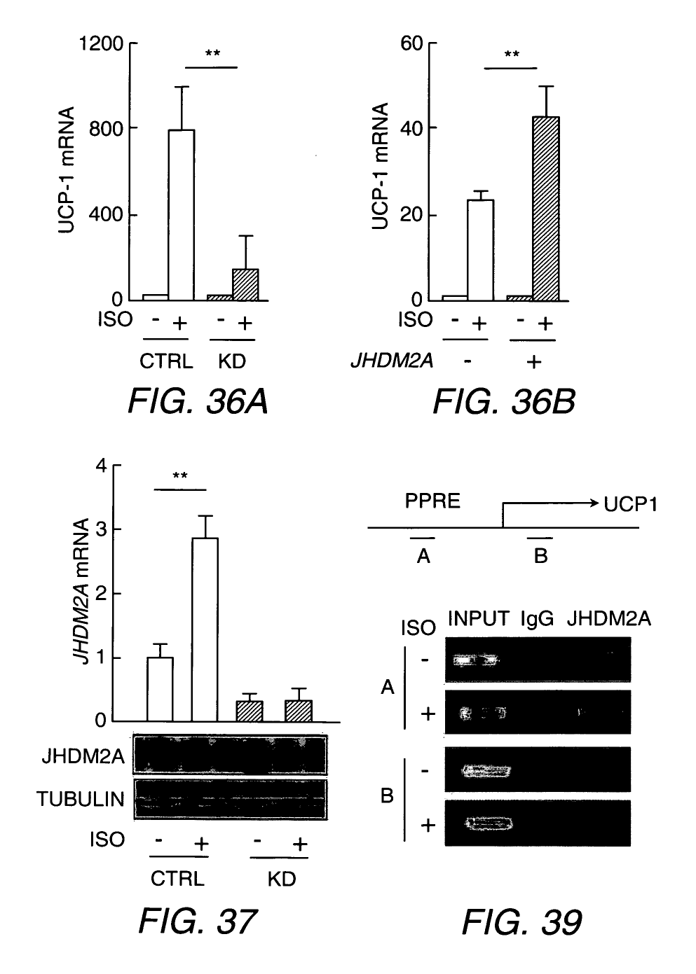

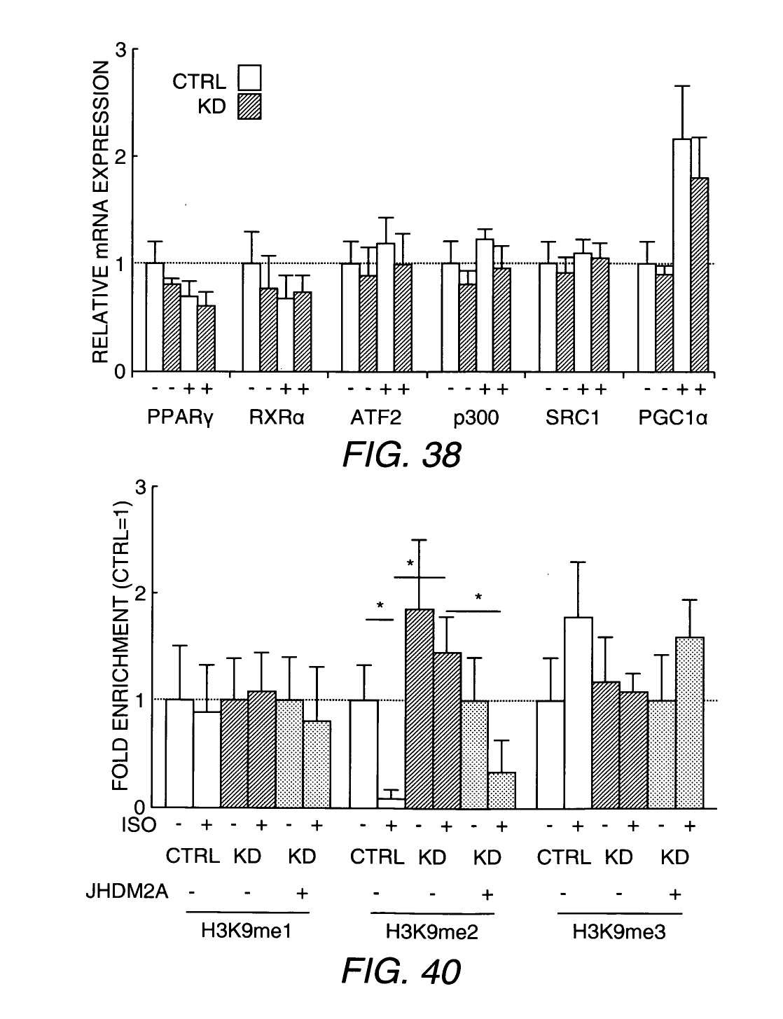

Given that cold-induced Ucp1 upregulation is intact in the PPARα-deficient mice (Kersten, et al., J. Clin. Invest. 103:1489 (1999)), the possibility was explored that Jhdm2a directly regulates Ucp1 expression in response to cold exposure. To ascertain that the effect of Jhdm2a on Ucp1 expression was cell-intrinsic, short-hairpin RNA (shRNA)-mediated was performed in the brown adipose cell line HIB1B using lentivirus-based shRNA. To generate a Jhdm2a knock-down HIB1B cell line, undifferentiated cells were infected with a lentiviral virus expressing a shRNA for Jhdm2a or control. The shRNA sequence for Jhdm2a was 5′-GCA GGT GTC ACT AGC CTT AAT-3′ (SEQ ID NO:76). The differentiation of HIB1B cells was performed as previously described (Ross, et al., Proc. Natl. Acad. Sci. USA 89:7561 (1992)). Although Jhdm2a knock-down did not affect HIB1B differentiation (data not shown), the differentiated knock-down cells had impaired Ucp1 activation by isoproterenol (ISO), a general β-adrenergic receptor agonist, see FIG. 36A, consistent with the result obtained in Jhdm2a knockout BAT (FIG. 34). To demonstrate that the effect was mediated by Jhdm2a, human JHDM2a was overexpressed by infecting cells with a retroviral vector expressing FLAG-tagged JHDM2A or a control before the cells were subjected to differentiation. Importantly, enforced overexpression of human JHDM2A in the knock-down cells partially rescued Ucp1 expression, see FIG. 36B. Both the messenger RNA and protein levels of Jhdm2a were upregulated in response to β-adrenergic receptor activation, see FIG. 37, supporting Jhdm2a as an integral component of the β-adrenergic signaling pathway. Similar to that observed in mouse brown fat tissues, the expression of the transcription factors and coactivators involved in Ucp1 activation was not significantly altered in the HIB1B knock-down cells, see FIG. 38. ISO-induced upregulation of Pgcla was also maintained in HIB1B knock-down cells, see FIG. 38.

The possibility that Jhdm2a directly regulates Ucp1 expression was subsequently examined. ChIP analysis demonstrated that Jhdm2a can bind to the Ucp1 enhancer region (Cassard-Doulcier, et al., Mol. Endocrinol. 7:497 (1993)) in a β-adrenergic receptor ligand-dependent manner. The binding was site-specific as Jhdm2a was not detected within the Ucp1 coding region, see FIG. 39, amplicon B). As expected, the H3K9me2 levels at the Jhdm2a binding site were specifically decreased in response to ISO treatment and this effect was abolished in the Jhdm2a knock-down cells, see FIG. 40. Notably, enforced overexpression of human JHDM2A in the knock-down cells partially rescued the increased H3K9me2 level in an ISO-dependent manner, see FIG. 40. Collectively, these results indicate that Jhdm2a expression is regulated by the β-adrenergic signaling pathway and that Jhdm2a contributes to Ucp1 activation by serving as a co-activator in response to β-adrenergic receptor activation.

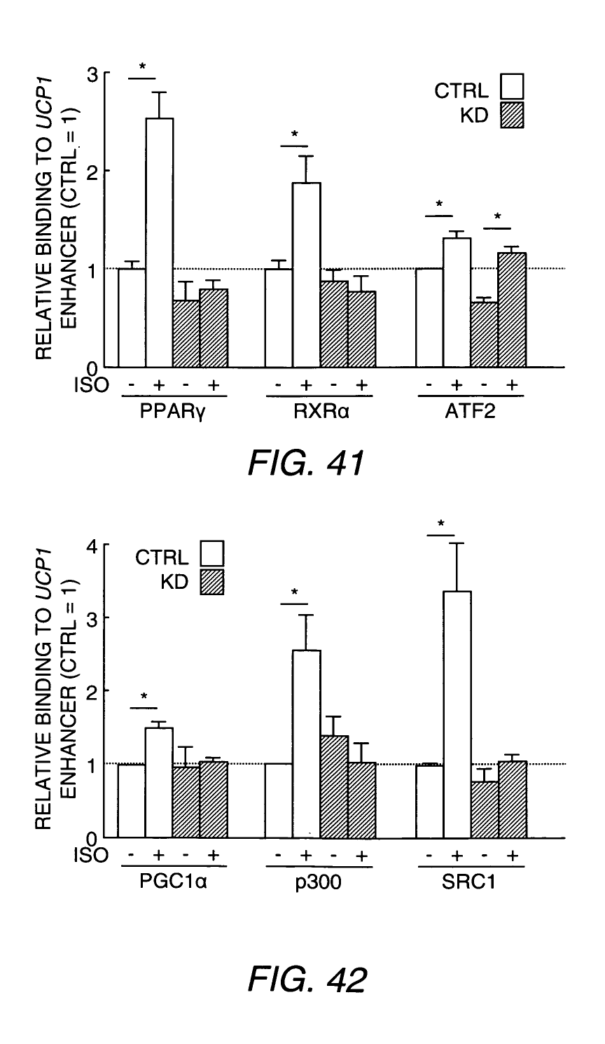

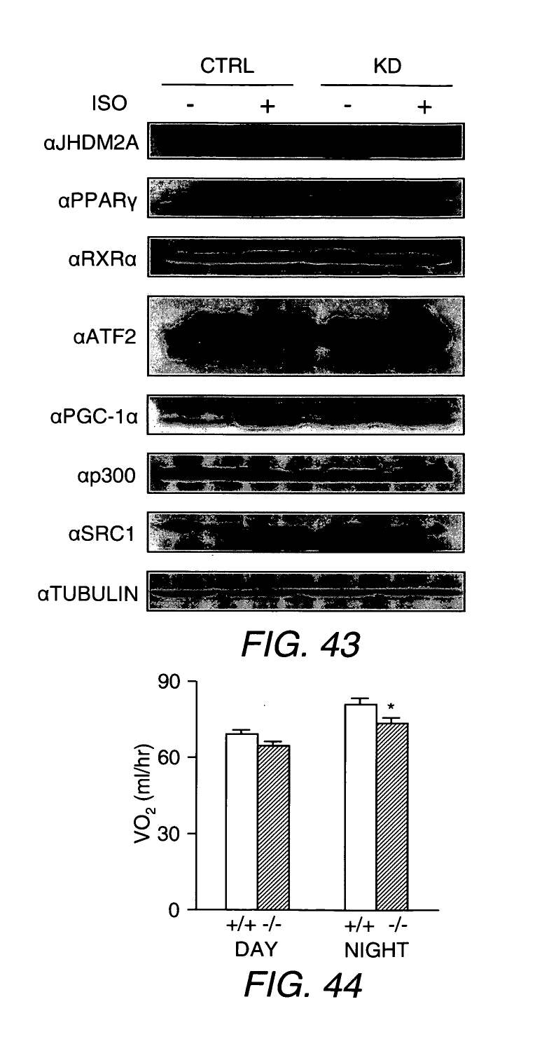

Previous studies have indicated that PPARγ- and Rxrα-mediated Ucp1 activation requires the recruitment of co-activators (Wang, et al., Cell Metab. 3, 111 (2006)). In addition to removing the repressive H3K9me2 mark at the Ucp1 enhancer, Jhdm2a could contribute to Ucp1 activation by affecting the recruitment of transcription factors and co-activators. To examine this possibility, the effect of the loss of Jhdm2a on the recruitment of transcription factors (PPARγ, Rxrα and Atf2) and co-activators (Pgc1α, CBP/p300 and Src1) was analyzed. It was found that the binding of PPARγ, Rxrα and Atf2 to the Ucp1 enhancer was increased in response to ISO treatment, see FIG. 41. However, the enhanced binding by PPARγ and Rxra disappeared in the Jhdm2a knock-down cells, see FIG. 41. Similar results were also observed for the co-activators, see FIG. 42. Given that Jhdm2a knock-down does not alter the protein levels of these transcription factors and co-activators, see FIG. 43, the simplest explanation of the results is that Jhdm2a facilitates their recruitment in response to β-adrenergic stimulation. These results, together with the previous demonstration that PPARγ recruitment to Ucp1 enhancer is blocked in pCip (also known as Ncoa3) and Src1 double knockout BAT (Lomax, et al., Endocrinology 148, 461 (2007)), indicate that the binding of transcription factors and coactivators to the Ucp1 enhancer affect one another. Collectively, the data herein indicate that Jhdm2a contributes to β-adrenergic-stimulated Ucp1 activation by maintaining a low level of H3K9me2 at the Ucp1 enhancer region, and by augmenting the recruitment of PPARγ and Rxrα and their co-activators to the Ucp1 enhancer element.

In the present study, PPARα and Ucp1 were identified as direct targets of Jhdm2a. Notably, the expression of these two genes, as well as Jhdm2a, is induced after β-adrenergic stimulation (Lowell, et al., Nature 404, 652 (2000); Lomax, et al., Endocrinology 148, 461 (2007)), see FIGS. 34, 35, and 37. These results indicate that Jhdm2a mediates β-adrenergic signaling on the basis of the systemic energy demand. Consistent with this, Jhdm2a knock-out mice and mice lacking β-adrenergic receptors have similar phenotypes that include defects in BAT function, cold intolerance, decreased oxygen consumption, see FIG. 44, and obesity without hyperphagia. Although the Jhdm2a deficiency did not affect metabolic hormone levels (Table 2), the obesity phenotype supports an important role for Jhdm2a in regulating systemic metabolic control including PPARα and β-adrenergic signaling pathways.

### Example 5

### Jhdm2a Knockdown in Myoblasts

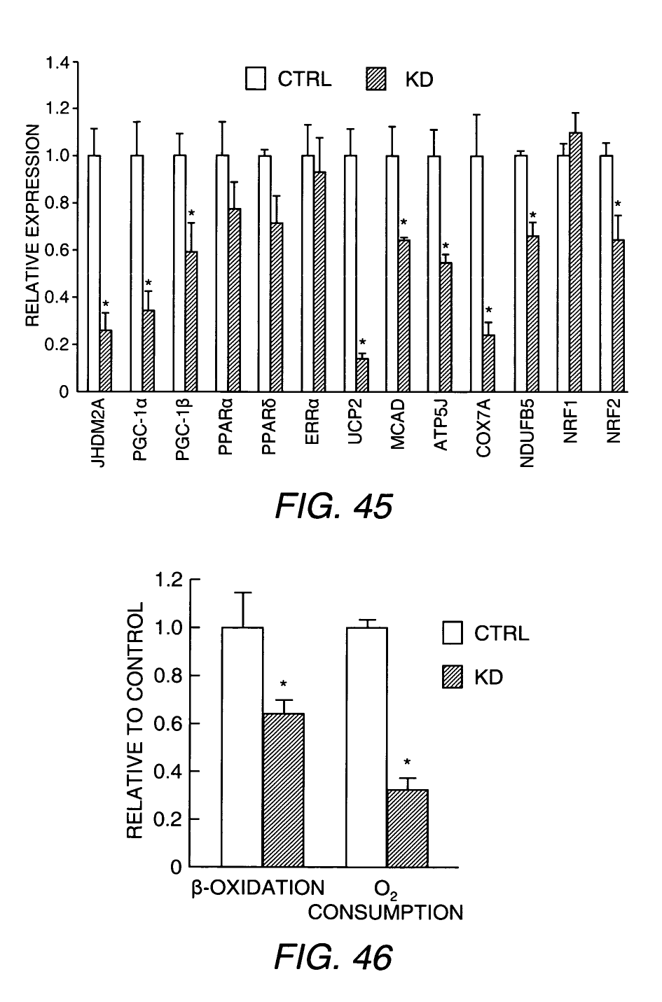

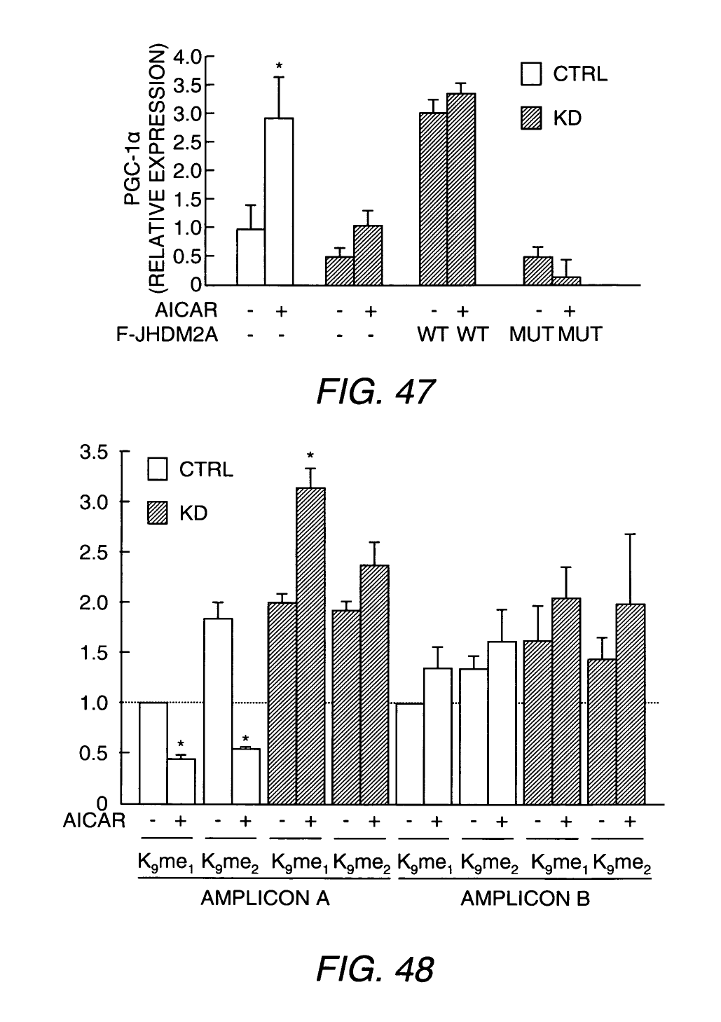

Jhdm2a knock-down was also carried out in C2C12 cells. To generate a Jhdm2a knock-down (KD) C2C12 cell line, myoblasts were infected with a lentiviral vector containing an RNAi construct for Jhdm2a or control, and were selected with puromycin. The RNAi construct for Jhdm2a was generated using the sequence: 5′-GCA GGT GTC ACT AGC CTT AAT-3′ (SEC) ID NO:76). Following puromycin selection, virally infected cell lines were grown to confluence and cultured in DMEM containing 2% horse serum to induce myotube differentiation. Consistent with a cell intrinsic effect, knock-down of Jhdm2a significantly reduced the expression levels of PGC-1α and its down stream targets, including UCP2, MCAD, ATP5J, and COX7A, see FIG. 45. In line with defective gene expression, knock-down of Jhdm2a in C2C12 cells also significantly reduced β-oxidation and O₂ consumption, see FIG. 46. To demonstrate that functional JHDM2A is required for AMPK-mediated PGC-1α induction, control and Jhdm2a knock-down C2C12 cells were treated with AICAR, an AMPK activation reagent. Results shown in FIG. 47 demonstrate that knock-down of Jhdm2a impaired AMPK-mediated PGC-1α induction.

Rescue experiments were performed to determine whether the demethylase activity of JHDM2A was required for AMPK-mediated PGC-1α induction by attempting to rescue PGC-1α induction using FLAG-tagged constructs encoding either a wild-type or an enzymatic defective mutant JHDM2A. For the rescue experiments, pcDNA3-human Jhdm2a or mutant jhdm2a (H1120Y) plasmid was transfected into Jhdm2a knock-down C2C12 myoblasts using LIPOFECTAMINE2000 (Invitrogen). Because the AMPK signaling is constitutively active in the primary Jhdm2a KO muscle cells (data not shown), Jhdm2a KD cells were chosen for the rescue experiment to avoid experimental bias due to pre-activation of AMPK. Importantly, over-expression of the wild-type human JHDM2A protein in the Jhdm2a knock-down C2C12 cells restored the expression of PGC-1α, see FIG. 47. However, over-expression of a catalytic JHDM2A (H1120Y) mutant failed to rescue PGC-1α expression in the KD cells, see FIG. 47.

The results herein indicate that JHDM2A directly contributes to the expression of PGC-1α in myoblasts. To determine whether PGC-1α is a direct target of JHDM2A, ChIP assays were performed. Ctrl or Jhdm2a KD C2C12 myotubes with or without AICAR treatment were used for ChIP analysis with histone modification-specific antibodies and anti-JHDM2A as described previously (Okada, Nature 450, 119 (2007)). The results demonstrated that JHDM2A is indeed localized to the promoter region (amplicon A), but not in the coding region (amplicon B), see FIG. 39. Importantly, the enrichment of JHDM2A at the promoter is lost upon Jhdm2a knock-down, confirming PGC-1α as a bona fide JHDM2A target (data not shown).

To further confirm that binding of JHDM2A to the PGC-1α promoter maintains a lower level of H3K9 methylation, the H3K9me1/2 levels in control and Jhdm2a KD C2C12 cells with or without the treatment of AICAR were analyzed. The results shown in FIG. 48 demonstrated that both H3K9me1 and H3K9me2 levels were decreased in response to AICAR-induced PGC-1α induction indicating JHDM2A-mediated H3K9 demethylation contributes to PGC-1α induction. Importantly, knockdown of Jhdm2a crippled AICAR-induced H3K9 demethylation at the PGC-1α promoter. The observed effect was promoter-specific as none of the treatment resulted in significant change in the H3K9 methylation levels in the coding region. Therefore, JHDM2A-mediated H3K9 demethylation in the PGC-1α promoter is an important event that links the cellular energy demand to the PGC-1α-dependent mitochondrial metabolism.

### Example 6

### JHDM2A SNPs

Single nucleotide polymorphisms in the human JHDM2A (JMJD1A) gene are listed in Table 3.

TABLE 3

| | Contig | mRNA | dbSNP rs# | | | dbSNP | Protein | Codon | Amino |
|---|---|---|---|---|---|---|---|---|---|
| Region | Position | position | cluster id | Heterozygosity | Function | allele | reside | position | Acid |
| 5′ region | 65482295 | | rs7598058 | N.D. | 5′ region | C/T | | | |
| | 65482295 | | rs59188381 | N.D. | 5′ region | C/T | | | |
| | 65482305 | | rs6547688 | N.D. | 5′ region | C/T | | | |
| | 65482305 | | rs61053559 | N.D. | 5′ region | C/T | | | |
| | 65482399 | | rs6547689 | N.D. | 5′ region | A/G | | | |
| | 65482399 | | rs61132402 | N.D. | 5′ region | A/G | | | |
| | 65482401 | | rs6547690 | N.D. | 5′ region | A/T | | | |
| | 65482401 | | rs56965415 | N.D. | 5′ region | A/T | | | |
| | 65483148 | | rs59717371 | N.D. | 5′ region | A/C | | | |
| | 65483251 | | rs10647570 | N.D. | 5′ region | —/TC | | | |
| | 65483252 | | rs60334362 | N.D. | 5′ region | —/CT | | | |
| | 65483329 | | rs10180036 | 0.5 | 5′ region | G/T | | | |
| | 65483329 | | rs58166018 | N.D. | 5′ region | G/T | | | |
| exon 1 | 65485111 | 245 | | | start codon | | | | 1 |
| intron 1 | 65484633 | | rs2271814 | 0.032 | intron | C/T | | | |
| intron 2 | 65485489 | | rs1914770 | N.D. | intron | C/G | | | |
| | 65485520 | | rs950438 | N.D. | intron | A/G | | | |
| | 65485727 | | rs4594437 | N.D. | intron | A/T | | | |
| | 65485742 | | rs949922 | N.D. | intron | C/T | | | |
| | 65485804 | | rs11888265 | N.D. | intron | A/G | | | |
| | 65486492 | | rs58219769 | N.D. | intron | A/T | | | |
| | 65486504 | | rs7574389 | 0.188 | intron | A/T | | | |
| | 65486504 | | rs60259213 | N.D. | intron | A/T | | | |
| | 65487014 | | rs56263644 | N.D. | intron | C/T | | | |
| | 65487063 | | rs58986545 | N.D. | intron | A/G | | | |
| | 65487288 | | rs10180819 | 0.014 | intron | C/T | | | |
| | 65487808 | | rs57291816 | N.D. | intron | C/T | | | |
| | 65488506 | | rs59274254 | N.D. | intron | C/T | | | |
| | 65489034 | | rs4832289 | 0.209 | intron | A/G | | | |
| | 65489034 | | rs58804028 | N.D. | intron | A/G | | | |
| | 65490557 | | rs13031596 | N.D. | intron | G/T | | | |
| | 65491171 | | rs58361202 | N.D. | intron | A/G | | | |
| | 65491874 | | rs34716822 | N.D. | intron | —/C | | | |
| | 65492305 | | rs12476666 | N.D. | intron | A/T | | | |
| | 65492305 | | rs61440850 | N.D. | intron | A/T | | | |
| | 65492419 | | rs11127040 | 0.24 | intron | C/G | | | |
| | 65492419 | | rs58377346 | N.D. | intron | C/G | | | |
| | 65492448 | | rs60304516 | N.D. | intron | A/G | | | |
| | 65492571 | | rs17027260 | 0.025 | intron | C/T | | | |
| intron 3 | 65493382 | | rs34043015 | N.D. | intron | —/C | | | |
| | 65493436 | | rs11682587 | 0.218 | intron | A/C | | | |
| | 65493436 | | rs60314420 | N.D. | intron | A/C | | | |
| | 65493999 | | rs34173194 | N.D. | intron | —/C | | | |
| | 65494064 | | rs57145635 | N.D. | intron | A/G | | | |
| intron 4 | 65494660 | | rs13431685 | N.D. | intron | A/G | | | |
| | 65494677 | | rs12714185 | 0.199 | intron | C/T | | | |
| | 65494677 | | rs59197130 | N.D. | intron | C/T | | | |
| | 65495273 | | rs35158232 | N.D. | intron | —/T | | | |
| | 65495365 | | rs6547691 | N.D. | intron | A/G | | | |
| | 65495365 | | rs59987532 | N.D. | intron | A/G | | | |
| | 65495484 | | rs55828608 | N.D. | intron | A/T | | | |
| | 65495801 | | rs58914241 | N.D. | intron | C/T | | | |
| | 65497761 | | rs28586601 | N.D. | intron | C/T | | | |
| | 65498037 | | rs34746063 | N.D. | intron | G/T | | | |
| intron 5 | 65498571 | | rs12714186 | N.D. | intron | A/G | | | |
| | 65498571 | | rs58813590 | N.D. | intron | A/G | | | |
| | 65498835 | | rs34893003 | N.D. | intron | —/G | | | |
| | 65499490 | | rs11431031 | N.D. | intron | —/T | | | |
| | 65499491 | | rs35089950 | N.D. | intron | —/T | | | |
| | 65499501 | | rs35497395 | N.D. | intron | —/T | | | |
| | 65499501 | | rs59291400 | N.D. | intron | —/T | | | |
| exon 6 | 65499528 | 824 | rs13424350 | N.D. | missense | A | Lys [K] | 1 | 194 |
| | | | | | contig | | | | |
| | | | | | reference | G | Glu [E] | | |
| | 65499582 | 878 | rs2030259 | 0.21 | missense | G | Val [V] | 1 | 212 |
| | | | | | contig | | | | |
| | | | | | reference | A | Ile [I] | | |
| | 65499582 | 878 | rs60083397 | N.D. | missense | G | Val [V] | 1 | 212 |
| | | | | | contig | | | | |
| | | | | | reference | A | Ile [I] | | |
| intron 6 | 65499649 | | rs10496321 | 0.12 | intron | G/T | | | |
| | 65499649 | | rs59093117 | N.D. | intron | G/T | | | |
| | 65499749 | | rs35768960 | N.D. | intron | —/G | | | |
| | 65499841 | | rs56347930 | N.D. | intron | C/G | | | |
| intron 8 | 65500981 | | rs62150267 | N.D. | intron | C/T | | | |
| | 65501073 | | rs6734709 | 0.069 | intron | A/G | | | |
| | 65501073 | | rs59698090 | N.D. | intron | A/G | | | |
| | 65501130 | | rs35229518 | N.D. | intron | —/G | | | |
| | 65501897 | | rs34118188 | N.D. | intron | —/T | | | |
| | 65502061 | | rs56377791 | N.D. | intron | —/T | | | |
| | 65502061 | | rs58539410 | N.D. | intron | A/T | | | |
| | 65502062 | | rs55866634 | N.D. | intron | A/G | | | |
| | 65502063 | | rs61155869 | N.D. | intron | —/G | | | |
| | 65502225 | | rs56169889 | N.D. | intron | C/T | | | |
| | 65502225 | | rs60842020 | N.D. | intron | C/T | | | |
| | 65502507 | | rs61158882 | N.D. | intron | A/G | | | |
| | 65503028 | | rs17027263 | 0.117 | intron | C/T | | | |
| | 65503928 | | rs35878123 | N.D. | intron | —/A | | | |
| | 65503930 | | rs34805982 | N.D. | intron | —/G | | | |
| | 65504892 | | rs34428050 | N.D. | intron | C/G | | | |
| | 65505011 | | rs59305606 | N.D. | intron | A/C | | | |
| | 65505996 | | rs56242451 | N.D. | intron | C/T | | | |
| | 65506091 | | rs11402914 | N.D. | intron | —/A | | | |
| | 65506479 | | rs12995989 | N.D. | intron | A/C | | | |
| | 65506479 | | rs58498681 | N.D. | intron | A/C | | | |
| | 65506481 | | rs56185141 | N.D. | intron | A/T | | | |
| | 65506481 | | rs57832914 | N.D. | intron | A/T | | | |
| | 65506486 | | rs6742018 | 0.107 | intron | G/T | | | |
| | 65506486 | | rs60247658 | N.D. | intron | —/G | | | |
| exon 9 | 65507190 | 1162 | rs12714187 | 0.271 | synonymous | A | Ala [A] | 3 | 306 |
| | | | | | contig | | | | |
| | | | | | reference | G | Ala [A] | | |
| | 65507190 | 1162 | rs61615233 | N.D. | synonymous | A | Ala [A] | 3 | 306 |
| | | | | | contig | | | | |
| | | | | | reference | G | Ala [A] | | |
| | 65507199 | 1171 | rs34507616 | 0.136 | synonymous | C | Pro [P] | 3 | 309 |
| | | | | | contig | | | | |
| | | | | | reference | A | Pro [P] | | |
| intron 9 | 65507340 | | rs34894003 | N.D. | intron | —/C | | | |
| | 65507508 | | rs11127041 | 0.079 | intron | A/T | | | |
| | 65507508 | | rs58814756 | N.D. | intron | A/T | | | |
| | 65508712 | | rs59183618 | N.D. | intron | C/T | | | |
| | 65509011 | | rs10541366 | N.D. | intron | —/AA | | | |
| | 65509012 | | rs34085670 | N.D. | intron | —/AA | | | |
| | 65509019 | | rs60791926 | N.D. | intron | —/AA | | | |
| | 65509020 | | rs59467248 | N.D. | intron | —/A | | | |
| | 65509157 | | rs57836876 | N.D. | intron | G/T | | | |
| | 65509243 | | rs58378769 | N.D. | intron | —/AT | | | |
| exon 10 | 65509766 | 1583 | rs34605051 | 0.167 | missense | C | Pro [P] | 1 | 447 |
| | | | | | contig | | | | |
| | | | | | reference | T | Ser [S] | | |
| intron 10 | 65510210 | | rs41318646 | 0.137 | intron | C/G | | | |
| | 65510210 | | rs56305001 | N.D. | intron | C/G | | | |
| | 65510358 | | rs1518811 | 0.263 | intron | A/T | | | |
| | 65510358 | | rs57439957 | N.D. | intron | A/T | | | |
| | 65511426 | | rs55778580 | N.D. | intron | G/T | | | |
| | 65511623 | | rs59507918 | N.D. | intron | —/TTAC | | | |
| | 65512391 | | rs58487331 | N.D. | intron | C/T | | | |
| intron 11 | 65514131 | | rs56989965 | N.D. | intron | A/C | | | |
| | 65514143 | | rs11127042 | 0.261 | intron | A/C | | | |
| | 65514143 | | rs58483755 | N.D. | intron | A/C | | | |
| | 65514216 | | rs57516528 | N.D. | intron | A/G | | | |
| | 65514338 | | rs9750710 | N.D. | intron | A/G | | | |
| | 65514350 | | rs9750711 | N.D. | intron | A/G | | | |
| | 65514392 | | rs9752698 | N.D. | intron | A/G | | | |
| | 65514400 | | rs9750715 | N.D. | intron | A/G | | | |
| | 65514402 | | rs9750725 | N.D. | intron | A/G | | | |
| | 65514404 | | rs13015458 | N.D. | intron | A/G | | | |
| | 65514412 | | rs55721077 | N.D. | intron | A/G | | | |
| | 65514412 | | rs62150268 | N.D. | intron | A/G | | | |
| | 65514422 | | rs62150269 | N.D. | intron | A/G | | | |
| | 65514493 | | rs56055358 | N.D. | intron | A/G | | | |
| | 65514695 | | rs13015954 | N.D. | intron | C/G | | | |
| | 65515236 | | rs36028213 | N.D. | intron | —/T | | | |
| | 65515236 | | rs59872014 | N.D. | intron | —/T | | | |
| | 65515656 | | rs58829418 | N.D. | intron | G/T | | | |
| | 65516006 | | rs35370675 | N.D. | intron | —/T | | | |
| | 65516452 | | rs34037894 | N.D. | intron | —/TG | | | |
| | 65517064 | | rs56300462 | N.D. | intron | C/T | | | |
| | 65517129 | | rs35007116 | N.D. | intron | —/A | | | |
| | 65517325 | | rs34163517 | N.D. | intron | —/A | | | |
| | 65517424 | | rs56228948 | N.D. | intron | A/C | | | |
| | 65517505 | | rs35749755 | N.D. | intron | —/CA | | | |
| | 65517506 | | rs56183122 | N.D. | intron | —/AC | | | |
| | 65517506 | | rs59034467 | N.D. | intron | —/AC | | | |
| | 65517765 | | rs34606174 | N.D. | intron | —/G | | | |
| | 65517805 | | rs57948637 | N.D. | intron | —/GTGTGTGT | | | |
| exon 12 | 65518008 | 2138 | rs35743643 | N.D. | frame shift | A | Met [M] | 1 | 632 |
| | | | | | contig | | | | |
| | | | | | reference | — | | | |
| intron 12 | 65519420 | | rs34748670 | N.D. | intron | —/T | | | |
| | 65519420 | | rs59867323 | N.D. | intron | —/T | | | |
| | 65519472 | | rs35120741 | N.D. | intron | —/CCT | | | |
| | 65519472 | | rs60288642 | N.D. | intron | —/CCT | | | |
| | 65519507 | | rs60539225 | N.D. | intron | C/T | | | |
| | 65520865 | | rs13399665 | 0.01 | intron | A/G | | | |
| exon 14 | 65521269 | 2373 | rs11677451 | N.D. | missense | A | Glu [E] | 2 | 710 |
| | | | | | contig | | | | |
| | | | | | reference | T | Val [V] | | |
| intron 14 | 65521505 | | rs2292887 | 0.5 | intron | A/G | | | |
| intron 15 | 65522633 | | rs59255035 | N.D. | intron | A/G | | | |
| | 65522648 | | rs11413436 | N.D. | intron | —/T | | | |
| | 65522649 | | rs34661548 | N.D. | intron | —/T | | | |
| | 65522656 | | rs35106652 | N.D. | intron | —/T | | | |
| | 65522774 | | rs60717666 | N.D. | intron | A/G | | | |
| exon 16 | 65523388 | 2719 | rs35269985 | 0.059 | synonymous | T | Ala [A] | 3 | 825 |
| | | | | | contig | | | | |
| | | | | | reference | C | Ala [A] | | |
| | 65523388 | 2719 | rs61748135 | N.D. | synonymous | T | Ala [A] | 3 | 825 |
| | | | | | contig | | | | |
| | | | | | reference | C | Ala [A] | | |
| intron 16 | 65523444 | | rs4832290 | 0.232 | intron | C/T | | | |
| | 65523444 | | rs57528778 | N.D. | intron | C/T | | | |
| exon 18 | 65525644 | 3053 | rs34673273 | 0.133 | synonymous | T | Leu [L] | 1 | 937 |
| | | | | | contig | | | | |
| | | | | | reference | C | Leu [L] | | |
| | 65525673 | 3082 | rs36101015 | 0.027 | synonymous | A | Leu [L] | 3 | 946 |
| | | | | | contig | | | | |
| | | | | | reference | T | Leu [L] | | |
| intron 18 | 65526299 | | rs3770058 | 0.443 | intron | C/G | | | |
| intron 19 | 65527371 | | rs2241803 | 0.499 | intron | C/T | | | |
| intron 20 | 65528360 | | rs35454242 | N.D. | intron | —/T | | | |
| intron 21 | 65529096 | | rs5832684 | N.D. | intron | —/A | | | |
| | 65529096 | | rs61503197 | N.D. | intron | —/A | | | |
| | 65529097 | | rs33958306 | N.D. | intron | —/A | | | |
| | 65529193 | | rs6719838 | N.D. | intron | C/G | | | |
| | 65530217 | | rs3755002 | 0.5 | intron | C/T | | | |
| | 65530217 | | rs57835500 | N.D. | intron | C/T | | | |
| | 65530371 | | rs56085540 | N.D. | intron | C/T | | | |
| | 65530495 | | rs55781109 | N.D. | intron | A/G | | | |
| | 65531636 | | rs1518812 | 0.108 | intron | C/T | | | |
| | 65531834 | | rs17027265 | 0.275 | intron | C/T | | | |
| | 65531834 | | rs61695154 | N.D. | intron | C/T | | | |
| | 65532021 | | rs61028062 | N.D. | intron | A/C | | | |
| intron 23 | 65532946 | | rs59338778 | N.D. | intron | —/GT | | | |
| | 65533692 | | rs1064100 | N.D. | intron | A/C | | | |
| | 65533756 | | rs1064101 | N.D. | intron | C/T | | | |
| | 65533809 | | rs17027270 | 0.428 | intron | G/T | | | |
| exon 24 | 65533948 | 3822 | rs17853822 | N.D. | nonsense | A | [Ter[*]] | 2 | 1193 |
| | | | | | contig | | | | |
| | | | | | reference | C | Ser [S] | | |
| intron 25 | 65535091 | | rs11883727 | N.D. | intron | A/C | | | |
| | 65535091 | | rs56673336 | N.D. | intron | A/C | | | |
| exon 26 | 65535173 | 4201 | rs34633400 | 0.025 | synonymous | G | Gly [G] | 3 | 1319 |
| | | | | | contig | | | | |
| | | | | | reference | C | Gly [G] | | |
| | 65535173 | 4201 | rs60789056 | N.D. | synonymous | G | Gly [G] | 3 | 1319 |
| | | | | | contig | | | | |
| | | | | | reference | C | Gly [G] | | |

The foregoing is illustrative of the present invention, and is not to be construed as limiting thereof. The invention is defined by the following claims, with equivalents of the claims to be included therein.

1. A transgenic mouse whose genome comprises a homozygous disruption of the Jhdm2a gene, wherein the disruption results in a deficiency in functional Jhdm2a gene expression in the mouse, and wherein the mouse exhibits one or more of the following characteristics relative to a wild-type mouse: (i) obesity, (ii) systemic insulin resistance, (iii) hyperinsulinemia, (iv) hyperlipidemia, (v) impaired energy expenditure, and (vi) reduced β-adrenergic receptor stimulated O₂ consumption.

2. The transgenic mouse of claim 1, wherein the disruption comprises the catalytic JmjC domain in exons 22, 23 and 24.

3. The transgenic mouse of claim 1, wherein the characteristic is obesity.

4. The transgenic mouse of claim 1, wherein the characteristic is impaired energy expenditure.

5. The transgenic mouse of claim 1, wherein the characteristic is reduced β-adrenergic receptor stimulated O₂ consumption.

6. The transgenic mouse of claim 1, wherein the transgenic mouse is a Jhdm2a knockout mouse.

7. The transgenic mouse of claim 6, wherein the transgenic mouse is a conditional knockout mouse.

8. An isolated transgenic cell from the transgenic mouse of claim 1.

9. The isolated cell of claim 8, wherein the cell is a brown adipose tissue cell, a white adipose tissue cell, a liver cell, or a skeletal muscle cell.

10. A cell culture comprising or produced by culturing the isolated cell of claim 8.

11. A transgenic mouse whose genome comprises a heterozygous disruption in the Jhdm2a gene, wherein the disruption results in a deficiency in functional Jhdm2a gene expression in the mouse, and wherein the mouse exhibits obesity on a high fat diet relative to a wild-type mouse.

12. The transgenic mouse of claim 11, wherein the mouse is obese.

13. A method of identifying a candidate compound for treating obesity, diabetes and/or metabolic syndrome, the method comprising:
administering a compound to the obese transgenic mouse of claim 3 after the onset of obesity; and
determining the body weight and/or level of obesity in the transgenic mouse,
wherein a reduction in body weight and/or obesity in the transgenic mouse administered the compound as compared with an untreated control mouse indicates that the compound is a candidate for treating obesity, diabetes and/or metabolic syndrome.

14. A method of identifying a candidate compound for preventing obesity, diabetes and/or metabolic syndrome, the method comprising:
administering a compound to the transgenic mouse of claim 3 prior to the onset of obesity and for a time sufficient for the onset of obesity in an untreated control mouse; and
determining the body weight and/or degree of obesity in the transgenic mouse administered the compound,
wherein a reduction in body weight and/or obesity in the transgenic mouse administered the compound relative to an untreated control mouse indicates that the compound is a candidate for preventing obesity, diabetes and/or metabolic syndrome.

15. A method of identifying a candidate compound for treating obesity, diabetes and/or metabolic syndrome, the method comprising:
administering a compound to the transgenic mouse of claim 4 after the onset of impaired energy expenditure; and
determining energy expenditure in the transgenic mouse,
wherein an increase in energy expenditure in the transgenic mouse administered the compound as compared with an untreated control mouse indicates that the compound is a candidate for treating obesity, diabetes and/or metabolic syndrome.

16. A method of identifying a candidate compound for preventing obesity, diabetes and/or metabolic syndrome, the method comprising:
administering a compound to the transgenic mouse of claim 4 prior to the onset of impaired energy expenditure and for a time sufficient for the onset of impairments in energy expenditure in an untreated control mouse; and
determining energy expenditure in the transgenic mouse,
wherein an increase in energy expenditure in the transgenic mouse administered the compound as compared with an untreated control mouse indicates that the compound is a candidate for preventing obesity, diabetes and/or metabolic syndrome.

17. The method of claim 15, wherein energy expenditure is determined by evaluating basal metabolic rate, physical activity and/or adaptive thermogenesis.

18. A method of identifying a candidate compound for treating obesity, diabetes and/or metabolic syndrome, the method comprising:
administering a compound to the transgenic mouse of claim 5 after the onset of reduced β-adrenergic receptor stimulated O₂ consumption; and
determining β-adrenergic receptor stimulated O₂ consumption by the transgenic mouse,
wherein an increase in β-adrenergic receptor stimulated O₂ consumption in the transgenic mouse administered the compound as compared with an untreated control mouse indicates that the compound is a candidate for treating obesity, diabetes and/or metabolic syndrome.

19. A method of identifying a candidate compound for preventing obesity, diabetes and/or metabolic syndrome, the method comprising:
administering a compound to the transgenic mouse of claim 5 prior to the onset of reduced β-adrenergic receptor stimulated O₂ consumption and for a time sufficient for the onset of reduced β-adrenergic receptor stimulated O₂ consumption in an untreated control mouse; and
determining β-adrenergic receptor stimulated O₂ consumption by the transgenic mouse,
wherein an increase in β-adrenergic receptor stimulated O₂ consumption in the transgenic mouse administered the compound relative to an untreated control mouse indicates that the compound is a candidate for preventing obesity, diabetes and/or metabolic syndrome.

20. The method of claim 18, wherein the determining step comprises administering a β-adrenergic receptor agonist to the transgenic mouse.

21. A method of identifying a candidate compound for preventing and/or treating obesity, diabetes and/or metabolic syndrome, the method comprising:
contacting a genetically modified cell having reduced expression of Jhdm2a with a compound,
determining energy expenditure and/or the activity of the AMPK-PGC-1α axis in the cell,
wherein increased energy expenditure and/or activity of the AMPK-PGC-1α axis in the cell contacted with the compound relative to an untreated control cell indicates that the compound is a candidate for preventing and/or treating obesity, diabetes and/or metabolic syndrome.

22. A method of identifying a candidate compound for preventing and/or treating obesity, diabetes and/or metabolic syndrome, the method comprising:
contacting a genetically modified cell having reduced expression of Jhdm2a with a compound,
determining PPARα signaling pathway activity and/or β-adrenergic receptor signaling pathway activity in the cell,
wherein an increase in PPARα signaling pathway activity and/or an increase in β-adrenergic receptor signaling pathway activity in the cell contacted with the compound relative to an untreated control cell indicates that the compound is a candidate for preventing and/or treating obesity, diabetes and/or metabolic syndrome.

23. The method of claim 21, wherein the cell comprises a homozygous disruption of the Jhdm2a gene.

24. The method of claim 23, wherein the cell is an isolated transgenic cell from a transgenic mouse whose genome comprises a homozygous disruption of the Jhdm2a gene, wherein the disruption results in a deficiency in functional Jhdm2a gene expression in the mouse, and wherein the mouse exhibits one or more of the following characteristics relative to a wild-type mouse: (i) obesity, (ii) systemic insulin resistance, (iii) hyperinsulinemia, (iv) hyperlipidemia, (v) impaired energy expenditure, and (vi) reduced β-adrenergic receptor stimulated O₂ consumption.

25. The method of claim 23, wherein the cell is a Jhdm2a knockdown cell.

26. A method of identifying a mammalian subject at increased risk for developing obesity, diabetes and/or metabolic syndrome, the method comprising detecting in the subject the presence of a genetic marker in the Jhmda2a gene, wherein the genetic marker is correlated with an increased risk of developing obesity, diabetes and/or metabolic syndrome, thereby identifying a mammalian subject having increased risk of developing obesity, diabetes and/or metabolic syndrome.

27. A method of identifying a mammalian subject at increased risk for developing obesity, diabetes and/or metabolic syndrome, the method comprising:
(a) correlating the presence of a genetic marker in the Jhdm2a gene with an increased risk of developing obesity, diabetes and/or metabolic syndrome; and
(b) detecting the presence of the genetic marker of (a) in a mammalian subject, thereby identifying a mammalian subject at increased risk for developing obesity, diabetes and/or metabolic syndrome.

28. A method of identifying a genetic marker in the Jhdma2 gene correlated with increased risk of a mammalian subject developing obesity, diabetes and/or metabolic syndrome, the method comprising:
(a) identifying a mammalian subject that has developed obesity, diabetes and/or metabolic syndrome;
(b) detecting in the mammalian subject the presence of a genetic marker in the Jhdm2a gene; and
(c) correlating the presence of the genetic marker of step (b) with the development of obesity, diabetes and/or metabolic syndrome, thereby identifying a genetic marker in the Jhdm2a gene correlated with increased risk of a mammalian subject developing obesity, diabetes and/or metabolic syndrome.

29. A method of identifying a genetic marker in the Jhdma2 gene correlated with increased risk of a mammalian subject developing obesity, diabetes and/or metabolic syndrome, the method comprising:
(a) identifying a mammalian subject that has developed obesity, diabetes and/or metabolic syndrome;
(b) determining the nucleotide sequence of the Jhdm2a gene of the mammalian subject of (a);
(c) comparing the nucleotide sequence of (b) with the wild-type nucleotide sequence of the Jhdm2a gene;
(d) detecting a genetic marker in the nucleotide sequence of (b); and
(e) correlating the presence of the genetic marker of step (b) with the development of obesity, diabetes and/or metabolic syndrome in the mammalian subject of (a).

30. The method of claim 26, wherein the genetic marker in the Jhdm2a gene is a single nucleotide polymorphism.
